SEQUENCE LISTING (11 sequences)

SEQ ID NO: 1 - DNA, 7412 bp, Homo sapiens
  source 1..7412: mol_type = genomic DNA
agtctgaatttagtagacagaagtcactaggaatgccttgacaggatcctgccttagctaaggctccctccagctgcagagggtgtttttgttagactcacacactgcgtgaaactgctcagaatagagccatgatctcaaccacgaaatgggaacttagattttggagaaactaacggggacggacttctttcctagcctgagtgttgagcagtgtcatgccttggcgtttcagctcctcgttgtctaggtggtgaaatgacagaactcattcgcttctttgattggtgattttgaaataatctttcatcaagttccatctcctttaccctcatatggaatatatctctctgtctgttgttaaactacgatgacatgtctgtagctatcagaaagagaagctgggaagaacatgtgacccactggatgggacagccttttaattctgatgatcgtaacacagcatgtcatcatggactagtagctgacagcttgcaggcaagtatggaaaaagatgcaactctaaatgtggaccgcaaagagaagtgtgtttcactacctgactgctgtcatggatcagagctgagagattttcctgggaggccaatgggtcatctttcaaaggatgtggacgaaaatgacagccatgaaggtgaagatcagtttctttctctggaagccagcacagaaacactagtgcatgtttctgatgaggataacaatgctgatttatgccttacagatgataaacaggttttaaatacccaagggcagaaaacatcaggccaacatatgatccaaggagcaggctccttagaaaaggcactgcccatcatacaaagtaaccaagtttcttctaactcctggggaatagctggtgaaactgaattagcactggtaaaagaaagtggggagagaaaagttactgactctataagtaaaagcctggagctttgcaatgaaataagcttaagtgaaataaaagatgcacccaaagtaaatgcagtggatactttgaacgtgaaagatattgcacctgagaaacaattgcttaactctgctgtaattgctcagcaacgaaggaaacctgacccccctaaagatgaaaatgaaagaagcacctgcaatgtagtacaaaatgagttcttggatactccttgcacaaacagaggactgccattattaaaaacagattttggaagctgccttctgcagcctccttcctgccccaatggaatgtcagctgaaaatggcctggagaagagtggtttttcacaacatcaaaacaaaagtccaccaaaggtcaaggcagaagatggcatgcagtgtttacaattaaaggagaccctggccacccaggaacccacagataaccaagtcagacttcgtaagagaaaggaaataagagaagatcgagatagggcgcggctggactccatggtgctgctgattatgaaactggaccagcttgatcaggacatagaaaatgccctcagcaccagctcctctccatcaggcacaccaacaaacctgcggcggcacgttcctgatctggaatcaggatctgaaagtggagcagataccatttcagtaaatcagacacgagtaaatttgtcttctgacactgagtccacggacctcccatcttccactccagtagccaattctggaaccaaacccaagactacggctattcaaggtatttcagagaaggaaaaggctgaaattgaagccaaggaagcttgtgattggctacgggcaactggtttcccccagtatgcacagctttatgaagatttcctgttccccatcgatatttccttggtcaagagagagcatgattttttggacagagatgccattgaggctctatgcaggcgtctaaatactttaaacaaatgtgcggtgatgaagctagaaattagtcctcatcggaaacgaagtgacgattcagacgaggatgagccttgtgccatcagtggcaaatggactttccaaagggacagcaagaggtggtcccggcttgaagagtttgatgtcttttctccaaaacaagacctggtccctgggtccccagacgactcccacccgaaggacggccccagccccggaggcacgctgatggacctcagcgagcgccaggaggtgtcttccgtccgcagcctcagcagcactggcagcctccccagccacgcgccccccagcgaggatgctgccaccccccggactaactccgtcatcagcgtttgctcctccagcaacttggcaggcaatgacgactctttcggcagcctgccctctcccaaggaactgtccagcttcagcttcagcatgaaaggccacgaaaaaactgccaagtccaagacgcgcagtctgctgaaacggatggagagcctgaagctcaagagctcccatcacagcaagcacaaagcgccctcaaagctggggttgatcatcagcgggcccatcttgcaagaggggatggatgaggagaagctgaagcagctcaactgcgtggagatctccgccctcaatggcaaccgcatcaacgtccccatggtacgaaagaggagcgtttccaactccacgcagaccagcagcagcagcagccagtcggagaccagcagcgcggtcagcacgcccagccctgttacgaggacccggagcctcagtgcgtgcaacaagcgggtgggcatgtacttagagggcttcgatcctttcaatcagtcaacatttaacaacgtggtggagcagaactttaagaaccgcgagagctacccagaggacacggtgttctacatccctgaagatcacaagcctggcactttccccaaagctctcaccaatggcagtttctccccctcggggaataacggctctgtgaactggaggacgggaagcttccacggccctggccacatcagcctcaggagggaaaacagtagcgacagccccaaggaactgaagagacgcaattcttccagctccatgagcagccgcctgagcatctacgacaacgtgccgggctccatcctctactccagttcaggggacctggcggatctggagaacgaggacatcttccccgagctggacgacatcctctaccacgtgaaggggatgcagcggatagtcaatcagtggtcggagaagttttctgatgagggagattcggactcagccctggactcggtctctccctgcccgtcctctccaaaacagatacacctggatgtggacaacgaccgaaccacacccagcgacctggacagcacaggcaactccctgaatgaaccggaagagccctccgagatcccggaaagaagggattctggggttggggcttccctaaccaggtccaacaggcaccgactgagatggcacagtttccagagctcacatcggccaagcctcaactctgtatcactacagattaactgccagtctgtggcccagatgaacctgctgcagaaatactcactcctaaagctaacggccctgctggagaaatacacaccttctaacaagcatggttttagctgggccgtgcccaagttcatgaagaggatcaaggttccagactacaaggaccggagtgtgtttggggtcccactgacggtcaacgtgcagcgcacaggacaaccgttgcctcagagcatccagcaggccatgcgatacctccggaaccattgtttggatcaggttgggctcttcagaaaatcgggggtcaagtcccggattcaggctctgcgccagatgaatgaaggtgccatagactgtgtcaactacgaaggacagtctgcttatgacgtggcagacatgctgaagcagtattttcgagatcttcctgagccactaatgacgaacaaactctcggaaacctttctacagatctaccaatatgtgcccaaggaccagcgcctgcaggccatcaaggctgccatcatgctgctgcctgacgagaaccgggaggttctgcagaccctgctttatttcctgagcgatgtcacagcagccgtaaaagaaaaccagatgaccccaaccaacctggccgtgtgcttagcgccttccctcttccatctcaacaccctgaagagagagaattcctctcccagggtaatgcaaagaaaacaaagtttgggcaaaccagatcagaaagatttgaatgaaaacctagctgccactcaagggctggcccatatgatcgccgagtgcaagaagcttttccaggttcccgaggaaatgagccgatgtcgtaattcctataccgaacaagagctgaagcccctcactctggaagcactcgggcacctgggtaatgatgactcagctgactaccaacacttcctccaggactgtgtggatggcctgtttaaagaagtcaaagagaagtttaaaggctgggtcagctactccacttcggagcaggctgagctgtcctataagaaggtgagcgaaggaccccctctgaggctttggaggtcagtcattgaagtccctgctgtgccagaggaaatcttaaagcgcctacttaaagaacagcacctctgggatgtagacctgttggattcaaaagtgatcgaaattctggacagccaaactgaaatttaccagtatgtccaaaacagtatggcacctcatcctgctcgagactacgttgttttaagaacctggaggactaatttacccaaaggagcctgtgcccttttactaacctctgtggatcacgatcgcgcacctgtggtgggtgtgagggttaatgtgctcttgtccaggtatttgattgaaccctgtgggccaggaaaatccaaactcacctacatgtgcagagttgacttaaggggccacatgccagaatggtacacaaaatcttttggacatttgtgtgcagctgaagttgtaaagatccgggattccttcagtaaccagaacactgaaaccaaagacaccaaatctaggtgatcactgaagcaacgcaaccgcttccaccaccatggtgtttgtttctagaacttttgccagtccttgaagaatgggttctgtgtctaatcctgaaacaaagaaaactacaagctggagtgtaggaattgactatagcaatttgatacatttttaaagctgcttcctgtttgttgagggtctgtattcatagaccttgactggaatatgtaagactgtgcaaaaaaaaaaaaaaaatttatgtgtattcttattcaaattgcttctgagaagcaaaactctttaaatacattatggaagataatgaagatacttcattctcttgtgatatcagtgtatgcgtacctgtgtcgctttatttgcagtgtgttgagggactggtgtatccactggaatagttggtactcttggatgtgttttctcaccaagatgagcaaagaaaggtttgcacagaggagtgtgaatgtgtgtttgttgctggctgaatggcaatagatgtctaaggtggattcagtgtctggcacactgagacacctccaagaaggagattgatgcatcaggttcagtttaacctggaatatctgactacccctgaatccacccagaaagggggcccaacacccttgtccatttatgggtattttttttcgaagttattaagcatattccttttccacgaacctcttctgtactttgattgtaataggttggctcttacacccattccaaatgcagtttatttttagacccgattgcaaatagtgatgtagttttaaccagtatggattagttcagggatgaactgctccctctagccttactggctctgatccacagggttttgttttgttttgttttgttttttgtttaagtcgagatataaaaactgaacacgataacacttactcttaaatcaagcatcaacactttttccctgttagaattctttgcatttttgtgtttgtaacagaaacgccttaagacactatgtttgggaatataggaaactatgtgtgtcccaaggaaatccctgtaaatttaactcacctacaaaaggctttttccccgcctttggttgttaacggcattcctgaaagccacatgtgtttattcattgggcttgttcttatcagcaaataggttttctggttttatgactttttgtcttattttatttttcctacatttctttttttttttttttcctttagaatgccctggaaatatatttaagtggtaatgaaaaatagtaatcatagtaaaacgcaacaagaagaaaaccaacccaaaccagtgaagttttttagaacctttagaagggtggtctttattcaggttttactgtaatggtaaggattgactcaagagacagtattagtaaatttattgtgtatggatcaaaagtgaataatgtatgaatgagagctgtaagaaggatttttattttgttataatttagttaccattttcagtgttatttcaaaggttctttgaagaattttggggcagggcatcagattagagttttaaaatttgagtattttggatatcagtgttcctcatgaagatatacatggatattcaattttgatggcttccagatttgtaagattgtatgttgtatataccattctattaagaaacatgtccactgtgctttcaaacatagataaagcatgataaagattattatttaagatatacttgtatttatacctcagatattcttttgggttttgtacctcaaggcttttttcttcttattgtaaatacactttacgtgaatacagtctaagtgaagaaaataaataaaaggaagaggtttataacttgctctatatctgtacagattataatcaataagtgcactattattaaatgtttaaagtaagggaaaagtctgggctgccttccttaatattgcatctcactcccacccttaaaaccacagattgcaaagcatagcattttagcatcaactacaatcaaaagagcgatttgctgaaggaaaaatcggactgcaaatcattccaaggccaaactgcaactgagccacccactcccaaacaggaaaccctggtgaaggttcaggaagcacggagattctctccaacaaaggtccagttaggaaacgacgctgagaggatgacgacaacgtgcaacagcagaaagatgcttgcaagcagagtcagggtcaccagtgaatgccacaaaagttctctttcccactgtttaatttgacaagagaagaatttgaaggatatgaacattttcaagaactctgctgaggtcacttagagcgccatcacaacttatttgtgtgactaattgcctagattgtaagctctttgagggcagggcttgtctcttacacatctttataatcccctgcagcggctttcagtattttgtacttgtaggcacctaataaatttattatttgctatactgaa

SEQ ID NO: 2 - DNA, 7246 bp, Homo sapiens
  source 1..7246: mol_type = genomic DNA
gctcactcatgattatcagacaacacattggcagtgagttcatctgctctactctgctttggaataacacatgtgattaaccaggtggtgaaatgacagaactcattcgcttctttgattggtgattttgaaataatctttcatcaagttccatctcctttaccctcatatggaatatatctctctgtctgttgttaaactacgatgacatgtctgtagctatcagaaagagaagctgggaagaacatgtgacccactggatgggacagccttttaattctgatgatcgtaacacagcatgtcatcatggactagtagctgacagcttgcaggcaagtatggaaaaagatgcaactctaaatgtggaccgcaaagagaagtgtgtttcactacctgactgctgtcatggatcagagctgagagattttcctgggaggccaatgggtcatctttcaaaggatgtggacgaaaatgacagccatgaaggtgaagatcagtttctttctctggaagccagcacagaaacactagtgcatgtttctgatgaggataacaatgctgatttatgccttacagatgataaacaggttttaaatacccaagggcagaaaacatcaggccaacatatgatccaaggagcaggctccttagaaaaggcactgcccatcatacaaagtaaccaagtttcttctaactcctggggaatagctggtgaaactgaattagcactggtaaaagaaagtggggagagaaaagttactgactctataagtaaaagcctggagctttgcaatgaaataagcttaagtgaaataaaagatgcacccaaagtaaatgcagtggatactttgaacgtgaaagatattgcacctgagaaacaattgcttaactctgctgtaattgctcagcaacgaaggaaacctgacccccctaaagatgaaaatgaaagaagcacctgcaatgtagtacaaaatgagttcttggatactccttgcacaaacagaggactgccattattaaaaacagattttggaagctgccttctgcagcctccttcctgccccaatggaatgtcagctgaaaatggcctggagaagagtggtttttcacaacatcaaaacaaaagtccaccaaaggtcaaggcagaagatggcatgcagtgtttacaattaaaggagaccctggccacccaggaacccacagataaccaagtcagacttcgtaagagaaaggaaataagagaagatcgagatagggcgcggctggactccatggtgctgctgattatgaaactggaccagcttgatcaggacatagaaaatgccctcagcaccagctcctctccatcaggcacaccaacaaacctgcggcggcacgttcctgatctggaatcaggatctgaaagtggagcagataccatttcagtaaatcagacacgagtaaatttgtcttctgacactgagtccacggacctcccatcttccactccagtagccaattctggaaccaaacccaagactacggctattcaaggtatttcagagaaggaaaaggctgaaattgaagccaaggaagcttgtgattggctacgggcaactggtttcccccagtatgcacagctttatgaagatttcctgttccccatcgatatttccttggtcaagagagagcatgattttttggacagagatgccattgaggctctatgcaggcgtctaaatactttaaacaaatgtgcggtgatgaagctagaaattagtcctcatcggaaacgaagtgacgattcagacgaggatgagccttgtgccatcagtggcaaatggactttccaaagggacagcaagaggtggtcccggcttgaagagtttgatgtcttttctccaaaacaagacctggtccctgggtccccagacgactcccacccgaaggacggccccagccccggaggcacgctgatggacctcagcgagcgccaggaggtgtcttccgtccgcagcctcagcagcactggcagcctccccagccacgcgccccccagcgaggatgctgccaccccccggactaactccgtcatcagcgtttgctcctccagcaacttggcaggcaatgacgactctttcggcagcctgccctctcccaaggaactgtccagcttcagcttcagcatgaaaggccacgaaaaaactgccaagtccaagacgcgcagtctgctgaaacggatggagagcctgaagctcaagagctcccatcacagcaagcacaaagcgccctcaaagctggggttgatcatcagcgggcccatcttgcaagaggggatggatgaggagaagctgaagcagctcaactgcgtggagatctccgccctcaatggcaaccgcatcaacgtccccatggtacgaaagaggagcgtttccaactccacgcagaccagcagcagcagcagccagtcggagaccagcagcgcggtcagcacgcccagccctgttacgaggacccggagcctcagtgcgtgcaacaagcgggtgggcatgtacttagagggcttcgatcctttcaatcagtcaacatttaacaacgtggtggagcagaactttaagaaccgcgagagctacccagaggacacggtgttctacatccctgaagatcacaagcctggcactttccccaaagctctcaccaatggcagtttctccccctcggggaataacggctctgtgaactggaggacgggaagcttccacggccctggccacatcagcctcaggagggaaaacagtagcgacagccccaaggaactgaagagacgcaattcttccagctccatgagcagccgcctgagcatctacgacaacgtgccgggctccatcctctactccagttcaggggacctggcggatctggagaacgaggacatcttccccgagctggacgacatcctctaccacgtgaaggggatgcagcggatagtcaatcagtggtcggagaagttttctgatgagggagattcggactcagccctggactcggtctctccctgcccgtcctctccaaaacagatacacctggatgtggacaacgaccgaaccacacccagcgacctggacagcacaggcaactccctgaatgaaccggaagagccctccgagatcccggaaagaagggattctggggttggggcttccctaaccaggtccaacaggcaccgactgagatggcacagtttccagagctcacatcggccaagcctcaactctgtatcactacagattaactgccagtctgtggcccagatgaacctgctgcagaaatactcactcctaaagctaacggccctgctggagaaatacacaccttctaacaagcatggttttagctgggccgtgcccaagttcatgaagaggatcaaggttccagactacaaggaccggagtgtgtttggggtcccactgacggtcaacgtgcagcgcacaggacaaccgttgcctcagagcatccagcaggccatgcgatacctccggaaccattgtttggatcaggttgggctcttcagaaaatcgggggtcaagtcccggattcaggctctgcgccagatgaatgaaggtgccatagactgtgtcaactacgaaggacagtctgcttatgacgtggcagacatgctgaagcagtattttcgagatcttcctgagccactaatgacgaacaaactctcggaaacctttctacagatctaccaatatgtgcccaaggaccagcgcctgcaggccatcaaggctgccatcatgctgctgcctgacgagaaccgggaggttctgcagaccctgctttatttcctgagcgatgtcacagcagccgtaaaagaaaaccagatgaccccaaccaacctggccgtgtgcttagcgccttccctcttccatctcaacaccctgaagagagagaattcctctcccagggtaatgcaaagaaaacaaagtttgggcaaaccagatcagaaagatttgaatgaaaacctagctgccactcaagggctggcccatatgatcgccgagtgcaagaagcttttccaggttcccgaggaaatgagccgatgtcgtaattcctataccgaacaagagctgaagcccctcactctggaagcactcgggcacctgggtaatgatgactcagctgactaccaacacttcctccaggactgtgtggatggcctgtttaaagaagtcaaagagaagtttaaaggctgggtcagctactccacttcggagcaggctgagctgtcctataagaaggtgagcgaaggaccccctctgaggctttggaggtcagtcattgaagtccctgctgtgccagaggaaatcttaaagcgcctacttaaagaacagcacctctgggatgtagacctgttggattcaaaagtgatcgaaattctggacagccaaactgaaatttaccagtatgtccaaaacagtatggcacctcatcctgctcgagactacgttgttttaagaacctggaggactaatttacccaaaggagcctgtgcccttttactaacctctgtggatcacgatcgcgcacctgtggtgggtgtgagggttaatgtgctcttgtccaggtatttgattgaaccctgtgggccaggaaaatccaaactcacctacatgtgcagagttgacttaaggggccacatgccagaatggtacacaaaatcttttggacatttgtgtgcagctgaagttgtaaagatccgggattccttcagtaaccagaacactgaaaccaaagacaccaaatctaggtgatcactgaagcaacgcaaccgcttccaccaccatggtgtttgtttctagaacttttgccagtccttgaagaatgggttctgtgtctaatcctgaaacaaagaaaactacaagctggagtgtaggaattgactatagcaatttgatacatttttaaagctgcttcctgtttgttgagggtctgtattcatagaccttgactggaatatgtaagactgtgcaaaaaaaaaaaaaaaatttatgtgtattcttattcaaattgcttctgagaagcaaaactctttaaatacattatggaagataatgaagatacttcattctcttgtgatatcagtgtatgcgtacctgtgtcgctttatttgcagtgtgttgagggactggtgtatccactggaatagttggtactcttggatgtgttttctcaccaagatgagcaaagaaaggtttgcacagaggagtgtgaatgtgtgtttgttgctggctgaatggcaatagatgtctaaggtggattcagtgtctggcacactgagacacctccaagaaggagattgatgcatcaggttcagtttaacctggaatatctgactacccctgaatccacccagaaagggggcccaacacccttgtccatttatgggtattttttttcgaagttattaagcatattccttttccacgaacctcttctgtactttgattgtaataggttggctcttacacccattccaaatgcagtttatttttagacccgattgcaaatagtgatgtagttttaaccagtatggattagttcagggatgaactgctccctctagccttactggctctgatccacagggttttgttttgttttgttttgttttttgtttaagtcgagatataaaaactgaacacgataacacttactcttaaatcaagcatcaacactttttccctgttagaattctttgcatttttgtgtttgtaacagaaacgccttaagacactatgtttgggaatataggaaactatgtgtgtcccaaggaaatccctgtaaatttaactcacctacaaaaggctttttccccgcctttggttgttaacggcattcctgaaagccacatgtgtttattcattgggcttgttcttatcagcaaataggttttctggttttatgactttttgtcttattttatttttcctacatttctttttttttttttttcctttagaatgccctggaaatatatttaagtggtaatgaaaaatagtaatcatagtaaaacgcaacaagaagaaaaccaacccaaaccagtgaagttttttagaacctttagaagggtggtctttattcaggttttactgtaatggtaaggattgactcaagagacagtattagtaaatttattgtgtatggatcaaaagtgaataatgtatgaatgagagctgtaagaaggatttttattttgttataatttagttaccattttcagtgttatttcaaaggttctttgaagaattttggggcagggcatcagattagagttttaaaatttgagtattttggatatcagtgttcctcatgaagatatacatggatattcaattttgatggcttccagatttgtaagattgtatgttgtatataccattctattaagaaacatgtccactgtgctttcaaacatagataaagcatgataaagattattatttaagatatacttgtatttatacctcagatattcttttgggttttgtacctcaaggcttttttcttcttattgtaaatacactttacgtgaatacagtctaagtgaagaaaataaataaaaggaagaggtttataacttgctctatatctgtacagattataatcaataagtgcactattattaaatgtttaaagtaagggaaaagtctgggctgccttccttaatattgcatctcactcccacccttaaaaccacagattgcaaagcatagcattttagcatcaactacaatcaaaagagcgatttgctgaaggaaaaatcggactgcaaatcattccaaggccaaactgcaactgagccacccactcccaaacaggaaaccctggtgaaggttcaggaagcacggagattctctccaacaaaggtccagttaggaaacgacgctgagaggatgacgacaacgtgcaacagcagaaagatgcttgcaagcagagtcagggtcaccagtgaatgccacaaaagttctctttcccactgtttaatttgacaagagaagaatttgaaggatatgaacattttcaagaactctgctgaggtcacttagagcgccatcacaacttatttgtgtgactaattgcctagattgtaagctctttgagggcagggcttgtctcttacacatctttataatcccctgcagcggctttcagtattttgtacttgtaggcacctaataaatttattatttgctatactgaa

SEQ ID NO: 3 - protein, 1528 aa, Homo sapiens
MSVAIRKRSWEEHVTHWMGQPFNSDDRNTACHHGLVADSLQASMEKDATLNVDRKEKCVSLPDCCHGSELRDFPGRPMGHLSKDVDENDSHEGEDQFLSLEASTETLVHVSDEDNNADLCLTDDKQVLNTQGQKTSGQHMIQGAGSLEKALPIIQSNQVSSNSWGIAGETELALVKESGERKVTDSISKSLELCNEISLSEIKDAPKVNAVDTLNVKDIAPEKQLLNSAVIAQQRRKPDPPKDENERSTCNVVQNEFLDTPCTNRGLPLLKTDFGSCLLQPPSCPNGMSAENGLEKSGFSQHQNKSPPKVKAEDGMQCLQLKETLATQEPTDNQVRLRKRKEIREDRDRARLDSMVLLIMKLDQLDQDIENALSTSSSPSGTPTNLRRHVPDLESGSESGADTISVNQTRVNLSSDTESTDLPSSTPVANSGTKPKTTAIQGISEKEKAEIEAKEACDWLRATGFPQYAQLYEDFLFPIDISLVKREHDFLDRDAIEALCRRLNTLNKCAVMKLEISPHRKRSDDSDEDEPCAISGKWTFQRDSKRWSRLEEFDVFSPKQDLVPGSPDDSHPKDGPSPGGTLMDLSERQEVSSVRSLSSTGSLPSHAPPSEDAATPRTNSVISVCSSSNLAGNDDSFGSLPSPKELSSFSFSMKGHEKTAKSKTRSLLKRMESLKLKSSHHSKHKAPSKLGLIISGPILQEGMDEEKLKQLNCVEISALNGNRINVPMVRKRSVSNSTQTSSSSSQSETSSAVSTPSPVTRTRSLSACNKRVGMYLEGFDPFNQSTFNNVVEQNFKNRESYPEDTVFYIPEDHKPGTFPKALTNGSFSPSGNNGSVNWRTGSFHGPGHISLRRENSSDSPKELKRRNSSSSMSSRLSIYDNVPGSILYSSSGDLADLENEDIFPELDDILYHVKGMQRIVNQWSEKFSDEGDSDSALDSVSPCPSSPKQIHLDVDNDRTTPSDLDSTGNSLNEPEEPSEIPERRDSGVGASLTRSNRHRLRWHSFQSSHRPSLNSVSLQINCQSVAQMNLLQKYSLLKLTALLEKYTPSNKHGFSWAVPKFMKRIKVPDYKDRSVFGVPLTVNVQRTGQPLPQSIQQAMRYLRNHCLDQVGLFRKSGVKSRIQALRQMNEGAIDCVNYEGQSAYDVADMLKQYFRDLPEPLMTNKLSETFLQIYQYVPKDQRLQAIKAAIMLLPDENREVLQTLLYFLSDVTAAVKENQMTPTNLAVCLAPSLFHLNTLKRENSSPRVMQRKQSLGKPDQKDLNENLAATQGLAHMIAECKKLFQVPEEMSRCRNSYTEQELKPLTLEALGHLGNDDSADYQHFLQDCVDGLFKEVKEKFKGWVSYSTSEQAELSYKKVSEGPPLRLWRSVIEVPAVPEEILKRLLKEQHLWDVDLLDSKVIEILDSQTEIYQYVQNSMAPHPARDYVVLRTWRTNLPKGACALLLTSVDHDRAPVVGVRVNVLLSRYLIEPCGPGKSKLTYMCRVDLRGHMPEWYTKSFGHLCAAEVVKIRDSFSNQNTETKDTKSR

SEQ ID NO: 4 - DNA, 885 bp, Homo sapiens
  source 1..885: mol_type = genomic DNA
atggcgatgagcagcggcggcagtggtggcggcgtcccggagcaggaggattccgtgctgttccggcgcggcacaggccagagcgatgattctgacatttgggatgatacagcactgataaaagcatatgataaagctgtggcttcatttaagcatgctctaaagaatggtgacatttgtgaaacttcgggtaaaccaaaaaccacacctaaaagaaaacctgctaagaagaataaaagccaaaagaagaatactgcagcttccttacaacagtggaaagttggggacaaatgttctgccatttggtcagaagacggttgcatttacccagctaccattgcttcaattgattttaagagagaaacctgtgttgtggtttacactggatatggaaatagagaggagcaaaatctgtccgatctactttccccaatctgtgaagtagctaataatatagaacagaatgctcaagagaatgaaaatgaaagccaagtttcaacagatgaaagtgagaactccaggtctcctggaaataaatcagataacatcaagcccaaatctgctccatggaactcttttctccctccaccaccccccatgccagggccaagactgggaccaggaaagccaggtctaaaattcaatggcccaccaccgccaccgccaccaccaccaccccacttactatcatgctggctgcctccatttccttctggaccaccaataattcccccaccacctcccatatgtccagattctcttgatgatgctgatgctttgggaagtatgttaatttcatggtacatgagtggctatcatactggctattatatgggtttcagacaaaatcaaaaagaaggaaggtgctcacattccttaaattaa

SEQ ID NO: 5 - protein, 294 aa, Homo sapiens
MAMSSGGSGGGVPEQEDSVLFRRGTGQSDDSDIWDDTALIKAYDKAVASFKHALKNGDICETSGKPKTTPKRKPAKKNKSQKKNTAASLQQWKVGDKCSAIWSEDGCIYPATIASIDFKRETCVVVYTGYGNREEQNLSDLLSPICEVANNIEQNAQENENESQVSTDESENSRSPGNKSDNIKPKSAPWNSFLPPPPPMPGPRLGPGKPGLKFNGPPPPPPPPPPHLLSCWLPPFPSGPPIIPPPPPICPDSLDDADALGSMLISWYMSGYHTGYYMGFRQNQKEGRCSHSLN

SEQ ID NO: 6 - DNA, 3754 bp, synthetic construct
  source 1..3754: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgttctagactatacagttgaagtcggaagtttacatacacttaagttggagtcattaaaactcgtttttcaactactccacaaatttcttgttaacaaacaatagttttggcaagtcagttaggacatctactttgtgcatgacacaagtcatttttccaacaattgtttacagacagattatttcacttataattcactgtatcacaattccagtgggtcagaagtttacatacactaagttgactgtgcctttaaacagcttggaaaattccagaaaatgatgtcatggctttagagaggatcctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggttctagacgtacggccaccatgggaattcttgagtgtatgtaaacttctgacccactgggaatgtgatgaaagaaataaaagctgaaatgaatcattctctctactattattctgatatttcacattcttaaaataaagtggtgatcctaactgacctaagacagggaatttttactaggattaaatgtcaggaattgtgaaaaagtgagtttaaatgtatttggctaaggtgtatgtaaacttccgacttcaactgtataggcatgcggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 7 - DNA, 5265 bp, synthetic construct
  source 1..5265: mol_type = other DNA
catgtcctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgtgtctagactgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgagaaggtaccggatccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaggaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctca

SEQ ID NO: 8 - DNA, 8543 bp, synthetic construct
  source 1..8543: mol_type = other DNA
ccaatgatacgcgtcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtacatcgacggtatcgggggagctcgcagggtctccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggctcttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtggacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaatatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcggacaggtaccaaaacaaatgttctcgtcacgtgggcatgaatctgatgctgtttccctgcagacaatgcgagagactgaatcagaattcaaatatctgcttcactcacggtgtcaaagactgtttagagtgctttcccgtgtcagaatctcaacccgtttctgtcgtcaaaaaggcgtatcagaaactgtgctacattcatcacatcatgggaaaggtgccagacgcttgcactgcttgcgacctggtcaatgtggacttggatgactgtgtttctgaacaataaatgacttaaaccaggtatgagtcggctggataaatctaaagtcataaacggcgctctggaattactcaatgaagtcggtatcgaaggcctgacgacaaggaaactcgctcaaaagctgggagttgagcagcctaccctgtactggcacgtgaagaacaagcgggccctgctcgatgccctggccatcgagatgctggacaggcatcatacccacttctgccccctggaaggcgagtcatggcaagactttctgcggaacaacgccaagtcattccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgagacaagcaattgagctgttcgaccggcagggagccgaacctgccttccttttcggcctggaactaatcatatgtggcctggagaaacagctaaagtgcgaaagcggcgggccggccgacgcccttgacgattttgacttagacatgctcccagccgatgcccttgacgactttgaccttgatatgctgcctgctgacgctcttgacgattttgaccttgacatgctccccgggtaaatgcatgaattcgatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgaatcaagctatcaagtgccacctgacgtctccctatcagtgatagagaagtcgacacgtctcgagctccctatcagtgatagagaaggtacgtctagaacgtctccctatcagtgatagagaagtcgacacgtctcgagctccctatcagtgatagagaaggtacgtctagaacgtctccctatcagtgatagagaagtcgacacgtctcgagctccctatcagtgatagagaaggtacgtctagaacgtctccctatcagtgatagagaagtcgacacgtctcgagctccctatcagtgatagagaaggtaccccctatataagcagagagatctgttcaaatttgaactgactaagcggctcccgccagattttggcaagattactaagcaggaagtcaaggacttttttgcttgggcaaaggtcaatcaggtgccggtgactcacgagtttaaagttcccagggaattggcgggaactaaaggggcggagaaatctctaaaacgcccactgggtgacgtcaccaatactagctataaaagtctggagaagcgggccaggctctcatttgttcccgagacgcctcgcagttcagacgtgactgttgatcccgctcctctgcgaccgctagcttcgatcaactacgcagacaggtaccaaaacaagtgttctcgtcacgtgggcattaatctgattctgtttccctgcagacaatgcgagagaatgaatcagaactcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgacttaagccaggtatggctgccgatggttatcttccagattggctcgaggacaaccttagtgaaggaattcgcgagtggtgggctttgaaacctggagcccctcaacccaaggcaaatcaacaacatcaagacaacgctagaggtcttgtgcttccgggttacaaataccttggacccggcaacggactcgacaagggggagccggtcaacgcagcagacgcggcggccctcgagcacgacaaagcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctctttaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctctagaactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggcctctcacaaagaaggagaggaccgtttctttcctttgtctggatctttaatttttggcaaacaaggtactggcagagacaacgtggatgcggacaaagtcatgataaccaacgaagaagaaattaaaactactaacccggtagcaacggagtcctatggacaagtggccacaaaccaccagagtgatgggactttggcggtgccttttaaggcacaggcgcagaccggttgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggtcaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaagtcgacttgcttgttaatcaataaaccgtttaattcgtttcagttgaactttggtctctgcgaagggcaattcgtttaaacctgcaggactagaggtcctgtattagaggtcacgtgagtgttttgcgacattttgcgacaccatgtggtcacgctgggtatttaagcccgagtgagcacgcagggtctccattttgaagcgggaggtttgaacgcgcagccgccaagccgaattctgcagatatcacatgtcctaggaactatcgatccatcacactggcggccgctcgactagagcggccgccaccgcggtggagctccagcttttgcggaccgaatcggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 9 - DNA, 896 bp, synthetic construct
  source 1..896: mol_type = other DNA
agtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcag

SEQ ID NO: 10 - DNA, 9137 bp, synthetic construct
  source 1..9137: mol_type = other DNA
catgtcctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgtgtctagactgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgagaaggtaccggatccatgtctgtagctatcagaaagagaagctgggaagaacatgtgacccactggatgggacagccttttaattctgatgatcgtaacacagcatgtcatcatggactagtagctgacagcttgcaggcaagtatggaaaaagatgcaactctaaatgtggaccgcaaagagaagtgtgtttcactacctgactgctgtcatggatcagagctgagagattttcctgggaggccaatgggtcatctttcaaaggatgtggacgaaaatgacagccatgaaggtgaagatcagtttctttctctggaagccagcacagaaacactagtgcatgtttctgatgaggataacaatgctgatttatgccttacagatgataaacaggttttaaatacccaagggcagaaaacatcaggccaacatatgatccaaggagcaggctccttagaaaaggcactgcccatcatacaaagtaaccaagtttcttctaactcctggggaatagctggtgaaactgaattagcactggtaaaagaaagtggggagagaaaagttactgactctataagtaaaagcctggagctttgcaatgaaataagcttaagtgaaataaaagatgcacccaaagtaaatgcagtggatactttgaacgtgaaagatattgcacctgagaaacaattgcttaactctgctgtaattgctcagcaacgaaggaaacctgacccccctaaagatgaaaatgaaagaagcacctgcaatgtagtacaaaatgagttcttggatactccttgcacaaacagaggactgccattattaaaaacagattttggaagctgccttctgcagcctccttcctgccccaatggaatgtcagctgaaaatggcctggagaagagtggtttttcacaacatcaaaacaaaagtccaccaaaggtcaaggcagaagatggcatgcagtgtttacaattaaaggagaccctggccacccaggaacccacagataaccaagtcagacttcgtaagagaaaggaaataagagaagatcgagatagggcgcggctggactccatggtgctgctgattatgaaactggaccagcttgatcaggacatagaaaatgccctcagcaccagctcctctccatcaggcacaccaacaaacctgcggcggcacgttcctgatctggaatcaggatctgaaagtggagcagataccatttcagtaaatcagacacgagtaaatttgtcttctgacactgagtccacggacctcccatcttccactccagtagccaattctggaaccaaacccaagactacggctattcaaggtatttcagagaaggaaaaggctgaaattgaagccaaggaagcttgtgattggctacgggcaactggtttcccccagtatgcacagctttatgaagatttcctgttccccatcgatatttccttggtcaagagagagcatgattttttggacagagatgccattgaggctctatgcaggcgtctaaatactttaaacaaatgtgcggtgatgaagctagaaattagtcctcatcggaaacgaagtgacgattcagacgaggatgagccttgtgccatcagtggcaaatggactttccaaagggacagcaagaggtggtcccggcttgaagagtttgatgtcttttctccaaaacaagacctggtccctgggtccccagacgactcccacccgaaggacggccccagccccggaggcacgctgatggacctcagcgagcgccaggaggtgtcttccgtccgcagcctcagcagcactggcagcctccccagccacgcgccccccagcgaggatgctgccaccccccggactaactccgtcatcagcgtttgctcctccagcaacttggcaggcaatgacgactctttcggcagcctgccctctcccaaggaactgtccagcttcagcttcagcatgaaaggccacgaaaaaactgccaagtccaagacgcgcagtctgctgaaacggatggagagcctgaagctcaagagctcccatcacagcaagcacaaagcgccctcaaagctggggttgatcatcagcgggcccatcttgcaagaggggatggatgaggagaagctgaagcagctcaactgcgtggagatctccgccctcaatggcaaccgcatcaacgtccccatggtacgaaagaggagcgtttccaactccacgcagaccagcagcagcagcagccagtcggagaccagcagcgcggtcagcacgcccagccctgttacgaggacccggagcctcagtgcgtgcaacaagcgggtgggcatgtacttagagggcttcgatcctttcaatcagtcaacatttaacaacgtggtggagcagaactttaagaaccgcgagagctacccagaggacacggtgttctacatccctgaagatcacaagcctggcactttccccaaagctctcaccaatggcagtttctccccctcggggaataacggctctgtgaactggaggacgggaagcttccacggccctggccacatcagcctcaggagggaaaacagtagcgacagccccaaggaactgaagagacgcaattcttccagctccatgagcagccgcctgagcatctacgacaacgtgccgggctccatcctctactccagttcaggggacctggcggatctggagaacgaggacatcttccccgagctggacgacatcctctaccacgtgaaggggatgcagcggatagtcaatcagtggtcggagaagttttctgatgagggagattcggactcagccctggactcggtctctccctgcccgtcctctccaaaacagatacacctggatgtggacaacgaccgaaccacacccagcgacctggacagcacaggcaactccctgaatgaaccggaagagccctccgagatcccggaaagaagggattctggggttggggcttccctaaccaggtccaacaggcaccgactgagatggcacagtttccagagctcacatcggccaagcctcaactctgtatcactacagattaactgccagtctgtggcccagatgaacctgctgcagaaatactcactcctaaagctaacggccctgctggagaaatacacaccttctaacaagcatggttttagctgggccgtgcccaagttcatgaagaggatcaaggttccagactacaaggaccggagtgtgtttggggtcccactgacggtcaacgtgcagcgcacaggacaaccgttgcctcagagcatccagcaggccatgcgatacctccggaaccattgtttggatcaggttgggctcttcagaaaatcgggggtcaagtcccggattcaggctctgcgccagatgaatgaaggtgccatagactgtgtcaactacgaaggacagtctgcttatgacgtggcagacatgctgaagcagtattttcgagatcttcctgagccactaatgacgaacaaactctcggaaacctttctacagatctaccaatatgtgcccaaggaccagcgcctgcaggccatcaaggctgccatcatgctgctgcctgacgagaaccgggaggttctgcagaccctgctttatttcctgagcgatgtcacagcagccgtaaaagaaaaccagatgaccccaaccaacctggccgtgtgcttagcgccttccctcttccatctcaacaccctgaagagagagaattcctctcccagggtaatgcaaagaaaacaaagtttgggcaaaccagatcagaaagatttgaatgaaaacctagctgccactcaagggctggcccatatgatcgccgagtgcaagaagcttttccaggttcccgaggaaatgagccgatgtcgtaattcctataccgaacaagagctgaagcccctcactctggaagcactcgggcacctgggtaatgatgactcagctgactaccaacacttcctccaggactgtgtggatggcctgtttaaagaagtcaaagagaagtttaaaggctgggtcagctactccacttcggagcaggctgagctgtcctataagaaggtgagcgaaggaccccctctgaggctttggaggtcagtcattgaagtccctgctgtgccagaggaaatcttaaagcgcctacttaaagaacagcacctctgggatgtagacctgttggattcaaaagtgatcgaaattctggacagccaaactgaaatttaccagtatgtccaaaacagtatggcacctcatcctgctcgagactacgttgttttaagaacctggaggactaatttacccaaaggagcctgtgcccttttactaacctctgtggatcacgatcgcgcacctgtggtgggtgtgagggttaatgtgctcttgtccaggtatttgattgaaccctgtgggccaggaaaatccaaactcacctacatgtgcagagttgacttaaggggccacatgccagaatggtacacaaaatcttttggacatttgtgtgcagctgaagttgtaaagatccgggattccttcagtaaccagaacactgaaaccaaagacaccaaatctaggtgatgtacaagtaggaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctca

SEQ ID NO: 11 - DNA, 5425 bp, synthetic construct
  source 1..5425: mol_type = other DNA
catgtcctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgtgtctagactgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgagaaggtaccggatccatggcgatgagcaatggcgatgagcagcggcggcagtggtggcggcgtcccggagcaggaggattccgtgctgttccggcgcggcacaggccagagcgatgattctgacatttgggatgatacagcactgataaaagcatatgataaagctgtggcttcatttaagcatgctctaaagaatggtgacatttgtgaaacttcgggtaaaccaaaaaccacacctaaaagaaaacctgctaagaagaataaaagccaaaagaagaatactgcagcttccttacaacagtggaaagttggggacaaatgttctgccatttggtcagaagacggttgcatttacccagctaccattgcttcaattgattttaagagagaaacctgtgttgtggtttacactggatatggaaatagagaggagcaaaatctgtccgatctactttccccaatctgtgaagtagctaataatatagaacaaaatgctcaagagaatgaaaatgaaagccaagtttcaacagatgaaagtgagaactccaggtctcctggaaataaatcagataacatcaagcccaaatctgctccatggaactcttttctccctccaccaccccccatgccagggccaagactgggaccaggaaagccaggtctaaaattcaatggcccaccaccgccaccgccaccaccaccaccccacttactatcatgctggctgcctccatttccttctggaccaccaataattcccccaccacctcccatatgtccagattctcttgatgatgctgatgctttgggaagtatgttaatttcatggtacatgagtggctatcatactggctattatatgggtttcagacaaaatcaaaaagaaggaaggtgctcacattccttaaattaaaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctca

(19) United States
(12) Patent Application Publication
(10) Pub. No.: US 2024/0279679 A1
(43) Pub. Date: Aug. 22, 2024

(54) COMPOSITIONS AND METHODS FOR TREATMENT OF SPINAL MUSCULAR ATROPHY

(71) Applicant: The University of Hong Kong, Hong Kong (CN)

(72) Inventors: Martin Cheung, Hong Kong (CN); Tianyuan Shi, Hong Kong (CN); Jessica Aijia Liu, Hong Kong (CN)

(21) Appl. No.: 18/170,296

(22) Filed: Feb. 16, 2023

(51) Int. Cl. C12N 15/86; A61P 21/00; C12N 15/11

(52) U.S. Cl. CPC C12N 15/86; A61P 21/00; C12N 15/11; C12N 2750/00043

(57) ABSTRACT

It has been discovered that overexpression of DLC1-i1 partly rescues neuronal formation and apoptosis of neuromuscular cells in subjects with spinal muscular atrophy (SMA). Since loss of DLC1-i1 confers specific MN defects in SMA, compositions and methods thereof for the treatment of SMA by delivery of AAV-SYN-DLC1-i1 into the neuronal population of subject with SMA are provided. The methods improve survival and restore locomotion ability to a greater extent than that of commercially-available compositions and methods. Methods including administration of both DLC1-i1 and SMN1 transgenes provide synergistic effects in improving the survival and enhancing locomotion ability in subject with SMA.

### REFERENCE TO SEQUENCE LISTING

The Sequence Listing submitted Feb. 16, 2023, as a text file named “UHK01295_ST25.txt,” created on Feb. 14, 2023, and having a size of 60,458 bytes is hereby incorporated by reference pursuant to 37 C.F.R. § 1.52(e)(5).

### FIELD OF THE INVENTION

The invention is generally in the field of gene therapy medicine for treating spinal muscular atrophy (SMA).

### BACKGROUND OF THE INVENTION

Muscular atrophy (MA) is a motor neuron degeneration disease caused by homozygous deletion or mutation of the survival motor neuron 1 (SMN1) gene and a retention of its homologue SMN2 gene, which predominantly encodes a truncated and unstable SMN2 protein together with a small fraction of the full-length, functional SMN2 protein. This results in overall deficiency of the ubiquitously expressed full-length SMN protein that causes progressive and selective degeneration of the spinal motor neuron axons and their innervation to muscle fibers. Consequently, patients with spinal Muscular atrophy SMA suffer from muscular atrophy and, in the most severe case, death.

It remains unclear how the loss of SMN in most tissues results in an motor neuron-specific phenotype. Current SMN gene therapy using a single, high titer intravenous bolus of AAV9-SMN results in reduced disease severity without marked improvement in locomotor activity. This highlights the importance of identifying alternative gene for more effective treatment of SMA.

Liver toxicity is one of the common side effects of gene therapies like AAV-SMN1 (Zolgensma) which rely on adeno-associated viruses (AAVs) as vectors to deliver therapeutic genes. AAVs are mostly directed at the liver and can lead to liver failure of patients who received high AAV doses. The FDA noted that about one third of the 500 patients who had received Zolgensma at the time had experienced a liver-related side effect. Besides liver toxicity, AAV-based gene therapies have also raised concerns that they might cause cancer.

Therefore, it is an object of the invention to provide reagents capable of reducing the severity of SMA and improving locomotor activity.

It is also an object of the invention to provide methods for providing improved long-term treatment for SMA without liver toxicity.

### SUMMARY OF INVENTION

It has been discovered that overexpression of the DLC1-i1 gene product in neuronal cells can greatly improve survival and locomotion ability in SMA patients. Compositions of AAV modified to deliver DLC1-i1 under the control of the human neuronal promoter SYN (AAV-SYN-DLC1-i1) and methods thereof for the treatment of SMA are provided.

Recombinant AAV viral particles including a vector encoding a Deleted in Liver Cancer 1-isoform 1 (DLC1-i1) transgene, or the expression product thereof are described. In some forms, the DLC1-i1 transgene is operably linked to a promoter selected from the group including the human synapsin 1 gene promoter (SYN), human β-glucuronidase promoter and a cytomegalovirus enhancer linked to a chicken β-actin promoter. In a preferred form, the DLC1-i1 transgene is operably linked to the human synapsin 1 gene promoter (SYN). An exemplary DLC1-i1 gene includes the nucleic acid sequence of SEQ ID NO: 1. An exemplary DLC1-i1 polypeptide includes an amino acid sequence of SEQ ID NO:2.

In some forms, the AAV viral particle includes an AAV1, AAV2, AAV3, AAV4, AAV5, AAV6, AAV7, AAVS, AAVrh8, AAV9, AAV10, AAVrh10, AAV11, or AAV12 serotype capsid. A preferred AAV is an AAV9 serotype capsid. Exemplary AAV serotype capsids include any one of AAV serotype Clades A-F. In some forms, the vector includes inverted terminal repeats (ITRs). Exemplary ITRs include AAV1, AAV2, AAV3, AAV4, AAV5, AAV6, AAV7, AAVS, AAVrh8, AAV9, AAV10, AAVrh10, AAV11, or AAV12 serotype ITRs. A preferred vector includes AAV serotype 2 ITRs.

Recombinant AAV viral particles including a vector encoding a Deleted in Liver Cancer 1-isoform 1 (DLC1-i1) transgene, and further including a survival motor neuron 1 (SMN1) transgene, are also described.

In some forms, the SMN1 transgene is within the same or different vector as the DLC1-i1 transgene. For example, in some forms, the SMN1 transgene is operably linked to the same or different promoter as the DLC1-i1 transgene.

In preferred forms, the SMN1 transgene is operably linked to the human synapsin 1 gene promoter (SYN).

Compositions for administration to a subject in vivo, are also described. Typically, the compositions include (a) recombinant AAV viral particles including a vector encoding a Deleted in Liver Cancer 1-isoform 1 (DLC1-i1) transgene, and further including a survival motor neuron 1 (SMN1) transgene; and (b) a pharmaceutically acceptable excipient for administration. Compositions including (c) a recombinant AAV viral particle including a survival motor neuron 1 (SMN1) transgene are also described.

In some forms, the SMN1 transgene is operably linked to a synapsin 1 gene promoter (SYN).

Typically, the recombinant AAV viral particles are in an amount of between about 2.5×10¹² genome copies and about 5×10¹³ genome copies. For example, in some forms, the recombinant AAV viral particles are in an amount of least 5×10¹³ genome copies per kg body weight of the subject.

Methods for treating spinal muscular atrophy (SMA) in a subject are also described. Typically, the methods include administering to the subject a first composition including recombinant AAV viral particles including a vector encoding a Deleted in Liver Cancer 1-isoform 1 (DLC1-i1) transgene. Generally, the methods ameliorate or minimize one or more symptoms of SMA in the subject. Exemplary symptoms include muscle wasting, inability to achieve motor milestones, inability to sit, inability to walk, paralysis, respiratory dysfunction, bulbar dysfunction, motor neuron cell loss and neuromuscular junction pathology.

Typically, the methods deliver a composition including at least 1×10¹² genome copies of recombinant AAV including a DLC1-i1 transgene. For example, in some forms the methods administer at least 3.5×10¹¹ genome copies per kg body weight of recombinant AAV viral particles including a DLC1-i1 transgene to the subject. In preferred forms, at least 10-30% of motor neurons in the lumbar, thoracic and cervical regions of the spinal cord of the subject are transduced by the recombinant AAV viral particles. In some forms, at least 30% of wild type level of DLC1-i1 is generated throughout the spinal cord. In some forms, the methods administer the composition to the subject via intravenous injection, or via direct injection into the spinal cord, or via intrathecal injection, or via intracisternal injection.

In some forms, the composition is administered to more than one location of the spinal cord or cisterna magna. In other forms, the composition is administered to more than one location of the spinal cord. In some forms, the composition is administered to one or more of a lumbar subarachnoid space, thoracic subarachnoid space and a cervical subarachnoid space of the spinal cord. In particular forms, the composition is administered to the cisterna magna.

In some forms, the methods include administering to the subject a second composition including a recombinant AAV viral particle including a survival motor neuron 1 (SMN1) transgene. In some forms, the SMN1 transgene is operably linked to a synapsin 1 gene promoter (SYN).

An exemplary SMN1 transgene includes the polynucleotide of SEQ ID NO:3. An exemplary SMN1 polypeptide includes the amino acid sequence of SEQ ID NO:4. In some forms, the second composition is administered to the subject at the same time as, before or after the first composition. Typically, the therapeutic effect of administering the first and second compositions to the subject is greater than the additive effects of administering the first composition alone or the second composition alone. In some forms, the methods administer at least 3.5×10¹¹ genome copies per kg body weight of recombinant AAV viral particles including a SMN1 transgene to the subject.

In some forms, the subject is a pediatric subject. In some forms, the subject is a young adult. In some forms, the subject has been diagnosed as having, or is suspected as having spinal muscular atrophy. For example, in some forms, the subject has a mutation in the endogenous DLC1-i1 gene, and/or the SMN-1 gene. In some forms, the subject has a partial deletion of the endogenous DLC1-i1 gene, and/or the SMN1 gene. In some forms, the subject has a complete deletion of the endogenous DLC1-i1 gene, and/or the SMN1 gene. Typically, the expression of the mutant DLC1-i1 gene, and/or the SMN-1 gene in spinal cord or brain of the subject is deficient compared to expression of DLC1-i1, and/or SMN-1 in a subject with a wild-type DLC1-i1 gene, and/or SMN-1 gene.

### BRIEF DESCRIPTION OF DRAWINGS

FIGS. 1A-1C. FIG. 1A is a schematic representation of the strategy used to differentiate human spinal cord motor neurons from hiPSCs/hESCs and the sample collection timepoints at different stages of MN differentiation including hESCs, EB, neural spheres (NSP) after neural induction, motor neuron progenitors (MNPs), MNs at 7 days post-differentiation of MNPs (7 D), and MNs 21 days post-differentiation of MNPs (21 D). FIG. 1B is a bar graph showing relative fold change (0-300) of DLC1-i1, i2, i3, 14, and i5 during different stages of MN differentiation including hESCs, EB, NSP, MNPs, 7 D, and 21 D as illustrated in FIG. 1A, measured by qRT-PCR. Data are represented as mean±SD. FIG. 1C is a line graph showing relative fold change compared to iPSCs (0.1-10,000) expression of DLC1 isoform 1 (DLC1-i1) with motor neuron marker ISLET1 during motor neuron differentiation during different stages of MN differentiation including hESCs, EB, NSP, MNPs, 7 D, and 21 D as illustrated in FIG. 1A, measured by qRT-PCR. Data are represented as mean±SD.

FIGS. 2A-2H. FIG. 2A is a schematic representation showing reprogramming of iPSCs from patients' and healthy individuals' urine by OKSM doxycycline-inducible lentivirus. FIG. 2B is a line graph showing action potential spikes in the mature MNs from healthy individuals' iPSCs. FIGS. 2C-2H are bar graphs showing relative fold change in mRNA expression levels measured by qRT-PCR compared to WT of SMN1 (FIG. 2C), DLC1-i1 (FIG. 2D), ChAT (FIG. 2E), ISLET1 (FIG. 2F), and TUJ1 (FIG. 2G) in 7-days post-differentiation of MNs from healthy individual or SMA patients (type I, type II-6, type II-9 and type III); and a bar graph showing TUJ1+ axonal length (in μm) in 7-days post-differentiation of MNs from healthy individual or SMA patients (type I, type II-6, type II-9 and type III) (FIG. 2H).

FIGS. 3A-3E. FIG. 3A is a schematic representation showing showed shRNA KD of DLC1-i1 strategies. FIGS. 3B-3E are bar graphs showing relative fold change in mRNA expression levels measured by qRT-PCR of DLC1-i1 (FIG. 3B), SOX2 (FIG. 3C), OLIG2 (FIG. 3D), and TUJ1 (FIG. 3E) in Scramble control and in two shRNA lentivirus mediated knockdown (KD1 and KD2) of DLC1-i1. Data are represented as mean±SD. Two-tailed unpaired t-test was used to calculate the statistical significance, and figures were labelled with * means P<0.01, ** means P<0.05, *** 20) means P<0.001, and **** means P<0.0001.

FIGS. 4A-4G are bar graphs showing relative fold change in mRNA expression levels measured by qRT-PCR of OLIG2 (FIG. 4A), ISLET1 (FIG. 4B), and TUJ1 (FIG. 4C) in Scramble control and in two shRNA lentivirus mediated knockdown (KD1 and KD2) of DLC1-i1 at 7 days of differentiating MNs; bar graph showing ChAT/DAPI (FIG. 4D), relative TUJ1+ axonal length compared to scramble control (FIG. 4E), OLIG2/DAPI (FIG. 4F), Cleaved caspase 3/DAPI (FIG. 4G) in Scramble control and KD1 and KD2 of DLC1-i1 at 21 days of differentiating MNs, based on the fluorescence images. Data are represented as mean±SD. Two-tailed unpaired t-test was used to calculate the statistical significance, and figures were labelled with * means P<0.01, ** means P<0.05, *** means P<0.001, **** means P<0.0001.

FIGS. 5A-5C. FIG. 5A is a schematic diagram of NMPs differentiation with expression of TEXT and SOX2 expression and NMOs differentiation from SMA patients' and healthy individual' urine-derived iPSCs. FIGS. 5B and 5C are bar graphs Cleaved caspase 3/DAPI (FIG. 5B) and OLIG2/DAPI (FIG. 5C) in WT or SMA patients' derived NMOs and their corresponding NMOs with overexpression of DLC1-i1 including Type I & Type I+OE, Type II & Type II+OE, and Type III & Type III+OE.

FIGS. 6A-6L are graphs showing probability of survival over a period of 60 days in WT control, SMA mice treated with AAV-SYN-SMN1 (SYN-SMN1), SMA mice treated with AAV-SYN-DLC1-i1 (SYN-DLC1-i1), and SMA mice treated with AAV-GFP, via intracerebroventricular (ICV) injection at a low dosage; Wt is 100% at 60 days, SYN-SMN1 is 0% at 35 days, SYN-DLC1-i1 is 0% at 14 days, GFP is 0% at 15 days (FIG. 6A), a medium dosage; Wt is 100% at 60 days, SYN-SMN1 is 0% at 41 days, SYN-DLC1-i1 is 0% at 49 days, GFP is 0% at 17 days (FIG. 6B), and a high dosage; Wt is 100% at 60 days, SYN-SMN1 is 0% at 26 days, SYN-DLC1-i1 is 0% at 49 days, GFP is 0% at 15 days (FIG. 6C), P<0.05 by Log-rank (mantel-cox) test; graphs showing righting time (0-120 seconds) over a period of 20 days in WT control, SMA mice treated with AAV-SYN-SMN1 (SYN-SMN1), SMA mice treated with AAV-SYN-DLC1-i1 (SYN-DLC1-i1), and SMA mice treated with AAV-GFP, via intracerebroventricular (ICV) injection at a low dosage; at day 1 lines correspond to (top to bottom) SYN-SMN1; SYN-DLC1-i1; GFP and Wt (FIG. 6D), a medium dosage; at day 1 lines correspond to (top to bottom) SYN-DLC1-i1; GFP; SYN-SMN1; and Wt (FIG. 6E), and a high dosage; at day 10 lines correspond to (top to bottom) GFP; SYN-DLC1-i1; SYN-SMN1; and Wt (FIG. 6F) P<0.05, two-way ANOVA, Mixed effect model (REML)); graphs showing ambulation score (0-3) over a period of 20 days in WT control, SMA mice treated with AAV-SYN-SMN1 (SYN-SMN1), SMA mice treated with AAV-SYN-DLC1-i1 (SYN-DLC1-i1), and SMA mice treated with AAV-GFP, via intracerebroventricular (ICV) injection at a low dosage; at day 8 lines correspond to (top to bottom) WT; SYN-DLC1-i1; SYN-SMN1; and GFP (FIG. 6G), a medium dosage; at day 9 lines correspond to (top to bottom) WT; SYN-DLC1-i1; SYN-SMN1; and GFP (FIG. 6H), and a high dosage; at day 9 lines correspond to (top to bottom) WT; SYN-DLC1-i1; SYN-SMN1; and GFP (FIG. 6I), P<0.05, unpaired two-tailed student's t-test; graphs showing four limbs hanging time over a period of 60 days in WT control, SMA mice treated with AAV-SYN-SMN1 (SYN-SMN1), SMA mice treated with AAV-SYN-DLC1-i1 (SYN-DLC1-i1), and SMA mice treated with AAV-GFP, via intracerebroventricular (ICV) injection at a low dosage; at day 15 lines correspond to (top to bottom) WT; SYN-DLC1-i1; SYN-SMN1; and GFP (FIG. 6J), a medium dosage; at day 15 lines correspond to (top to bottom) WT; SYN-DLC1-i1; SYN-SMN1; and GFP (FIG. 6K), and a high dosage; at day 15 lines correspond to (top to bottom) WT; SYN-DLC1-i1; SYN-SMN1; and GFP (FIG. 6L) P<0.05, unpaired two-tailed student's t-test. FIGS. 6M and 6N are schematics showing AAV expression vectors for delivery of transgenes DLC1-i1 (FIG. 6M) and SMN1 (FIG. 6N), respectively, showing the relative orientation of the transgenes flanked by the corresponding restriction enzyme-cut sites used for cloning, and other functional components of the vectors.

FIGS. 7A-7D are graphs showing probability of survival over a period of 60 days in SMN^{+/+} WT mice, and SMN^{-/-} mice treated with GFP, with AAV-PHP.eB SYN-SMN1 middle dosage, or with SYN-SMN1+SYN-DLC1-i1 (P<0.05 by Log-rank (mantel-cox) test); Wt is 100% at 60 days, SYN-SMN1+SYN-DLC1-i1 is 0% at 49 days, SYN-SMN1 Middle is 0% at 41 days, GFP is 0% at 15 days (FIG. 7A); graph showing ambulation score (0-3) over a period of 20 days in SMN^{+/+} WT mice, and SMN^{-/-} mice treated with GFP, with AAV-PHP.eB SYN-SMN1 middle dosage, or with SYN-SMN1+SYN-DLC1-i1 (P<0.05, unpaired two-tailed student's t-test); at day 9 lines correspond to (top to bottom) WT; SYN-SMN1+SYN-DLC1-i1; SYN-SMN1 Middle; and GFP (FIG. 7B); graph showing four limbs hanging time over a period of 60 days in SMN^{+/+} WT mice, and SMN^{-/-} mice treated with GFP, with AAV-PHP.eB SYN-SMN1 middle dosage, or with SYN-SMN1+SYN-DLC1-i1 (P<0.05, unpaired two-tailed student's t-test); at day 14 lines correspond to (top to bottom) WT; SYN-SMN1+DLC1-i1; SYN-SMN1 Middle; and Smn+GFP (FIG. 7C); and graph showing righting time (0-120 seconds) over a period of 20 days in SMN^{+/+} WT mice, and SMN^{-/-} mice treated with GFP, with AAV-PHP.eB SYN-SMN1 middle dosage, or with SYN-SMN1+SYN-DLC1-i1 (P<0.05, two-way ANOVA, Mixed effect model (REML)); at day 10 lines correspond to (top to bottom); GFP; SYN-SMN1 Middle; SYN-SMN1+DLC1-i1; and WT (FIG. 7D).

### DETAILED DESCRIPTION OF THE INVENTION

### I. Definitions

“Encoding” or “encode” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein, the term “locus” is the specific physical location of a DNA sequence (e.g., of a gene) on a chromosome. It is understood that a locus of interest can not only qualify a nucleic acid sequence that exists in the main body of genetic material (i.e., in a chromosome) of a cell but also a portion of genetic material that can exist independently to said main body of genetic material such as plasmids, episomes, virus, transposons or in organelles such as mitochondria as non-limiting examples.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell. An “isolated nucleic acid” refers to a nucleic acid segment or fragment which has been separated from sequences which flank it in a naturally occurring state, e.g., a DNA fragment which has been removed from the sequences which are normally adjacent to the fragment, i.e., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (i.e., as a cDNA or a genomic or cDNA fragment produced by PCR or restriction enzyme digestion) independent of other sequences. It also includes: a recombinant DNA which is part of a hybrid gene encoding additional polypeptide sequence, complementary DNA (cDNA), linear or circular oligomers or polymers of natural and/or modified monomers or linkages, including deoxyribonucleosides, ribonucleosides, substituted and alpha-anomeric forms thereof, peptide nucleic acids (PNA), locked nucleic acids (LNA), phosphorothioate, methylphosphonate, and the like.

In the context of cells, the term “isolated” also refers to a cell altered or removed from its natural state. That is, the cell is in an environment different from that in which the cell naturally occurs, e.g., separated from its natural milieu such as by concentrating to a concentration at which it is not found in nature. “Isolated cell” is meant to include cells that are within samples that are substantially enriched for the cell of interest and/or in which the cell of interest is partially or substantially purified.

As used herein, the term “percent (%) sequence identity” is defined as the percentage of nucleotides or amino acids in a candidate sequence that are identical with the nucleotides or amino acids in a reference nucleic acid sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

The terms “treat” or “treatment” of a disease, disorder or condition refer to improving one or more symptoms or the general condition of a subject having the disease. Treating the disease or condition includes ameliorating at least one symptom of the particular disease or condition, even if the underlying pathophysiology is not affected, such as treating the pain of a subject by administration of an analgesic agent even though such agent does not treat the cause of the pain. Desirable effects of treatment include decreasing the rate of disease progression, ameliorating or palliating the disease state, and remission or improved prognosis. In the case of cancer, “treating” the cancer refers to inhibiting proliferation or metastasis of a cancer or tumor cells. In some forms, treatment leads to stasis, partial or complete remission of a tumor or inhibit metastatic spreading of the tumor. In the case of an infectious disease, “treating” the infectious disease means reducing the load of the infections agent in the subject. In some forms, the load is viral load, and reducing the viral load means, for example, reducing the number of cells infected with influenza virus or coronavirus, reducing the rate of replication of influenza virus or coronavirus, reducing the number of new virions produced or reducing the number of total viral genome copies in a cell, as compared to an untreated subject. In some forms, the load is influenza virus, or coronavirus, as compared to an untreated subject, or as compared to a healthy, uninfected subject.

The terms “effective amount” or “therapeutically effective amount” mean a dosage or other amount of an active agent that produces some desired effect at a reasonable benefit/risk ratio applicable to any medical treatment or diagnosis. Typically, an amount of an agent is therapeutically effective if it is sufficient to alleviate one or more symptoms of a disorder, disease, or condition being treated, or to otherwise provide a desired pharmacologic and/or physiologic effect. The precise dosage will vary according to a variety of factors such as subject-dependent variables (e.g., age, immune system health, etc.), the disease or disorder being treated, as well as the route of administration and the pharmacokinetics of the agent being administered. One of ordinary skill in the art may empirically determine the effective amount of a particular compound without necessitating undue experimentation.

The terms “pharmaceutically acceptable” or “biocompatible” refer to compositions, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The phrase “pharmaceutically acceptable carrier” refers to pharmaceutically acceptable materials, compositions or vehicles, such as a liquid or solid filler, diluent, solvent or encapsulating material involved in carrying or transporting any subject composition, from one organ, or portion of the body, to another organ, or portion of the body. Each carrier must be “acceptable” in the sense of being compatible with the other ingredients of a subject composition and not injurious to the patient. The term “pharmaceutically acceptable salt” is art-recognized, and includes relatively non-toxic, inorganic and organic acid addition salts of compounds. Examples of pharmaceutically acceptable salts include those derived from mineral acids, such as hydrochloric acid and sulfuric acid, and those derived from organic acids, such as ethanesulfonic acid, benzenesulfonic acid, and p-toluenesulfonic acid. Examples of suitable inorganic bases for the formation of salts include the hydroxides, carbonates, and bicarbonates of ammonia, sodium, lithium, potassium, calcium, magnesium, aluminum, and zinc. Salts may also be formed with suitable organic bases, including those that are non-toxic and strong enough to form such salts. For purposes of illustration, the class of such organic bases may include mono-, di-, and trialkylamines, such as methylamine, dimethylamine, and triethylamine; mono-, di- or trihydroxyalkylamines such as mono-, di-, and triethanolamine; amino acids, such as arginine and lysine; guanidine; N-methylglucosamine; N-methylglucamine; L-glutamine; N-methylpiperazine; morpholine; ethylenediamine; and N-benzylphenethylamine.

The term “biodegradable” generally refers to a material that will degrade or erode under physiological conditions to smaller units or chemical species that are capable of being metabolized, eliminated, or excreted by the body. The degradation time of a material is a function of composition and morphology of the material.

The terms “inhibit” or “reduce” generally mean to reduce or decrease in activity and quantity. This can be a complete inhibition or reduction in activity or quantity, or a partial inhibition or reduction. Inhibition or reduction can be compared to a control or to a standard level. Inhibition can be 5, 10, 25, 50, 75, 80, 85, 90, 95, 99, or 100%, or an integer there between. In some forms, the inhibition and reduction are compared at mRNAs, proteins, cells, tissues and organs levels.

The terms “prevent”, “prevention” or “preventing” mean to administer a composition or method to a subject or a system at risk for or having a predisposition for one or more symptom caused by a disease or disorder, to decrease the likelihood the subject will develop one or more symptoms of the disease or disorder, or to reduce the severity, duration, or time of onset of one or more symptoms of the disease or disorder.

The terms “bioactive agent” and “active agent”, as used interchangeably include, without limitation, physiologically or pharmacologically active substances that act locally or systemically in the body. A bioactive agent is a substance used for the treatment (e.g., therapeutic agent), prevention (e.g., prophylactic agent), diagnosis (e.g., diagnostic agent), cure or mitigation of disease or illness, a substance which affects the structure or function of the body, or pro-drugs, which become biologically active or more active after they have been placed in a predetermined physiological environment.

As used herein, “transformed,” “transduced,” and “transfected” encompass the introduction of a nucleic acid or other material into a cell by one of a number of techniques known in the art.

“Endogenous” refers to any material from or produced inside an organism, cell, tissue or system. “Exogenous” refers to any material introduced from or produced outside an organism, cell, tissue or system.

A “vector” is a composition of matter which includes an isolated nucleic acid and which can be used to deliver the isolated nucleic acid to the interior of a cell. Examples of vectors include but are not limited to, linear polynucleotides, polynucleotides associated with ionic or amphiphilic compounds, plasmids, and viruses. Thus, the term “vector” encompasses an autonomously replicating plasmid or a virus. The term is also construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus (AAV) vectors, retroviral vectors, and the like.

As used herein, “subject” includes, but is not limited to, animals, plants, parasites and any other organism or entity. The subject can be a vertebrate, more specifically a mammal (e.g., a human, horse, pig, rabbit, dog, sheep, goat, non-human primate, cow, cat, guinea pig or rodent), a fish, a bird or a reptile or an amphibian. The subject can be an invertebrate, more specifically an arthropod (e.g., insects and crustaceans). The term does not denote a particular age or sex. Thus, adult and newborn subjects, as well as fetuses, whether male or female, are intended to be covered. A patient refers to a subject afflicted with a disease or disorder. The term “patient” includes human and veterinary subjects. In some forms, the subject can be any organism in which the disclosed method can be used to genetically modify the organism or cells of the organism.

The terms “protein” “polypeptide” or “peptide” refer to a natural or synthetic molecule including two or more amino acids linked by the carboxyl group of one amino acid to the alpha amino group of another.

The term “polynucleotide” or “nucleic acid” or “nucleic acid sequence” refers to a natural or synthetic molecule including two or more nucleotides linked by a phosphate group at the 3′ position of one nucleotide to the 5′ end of another nucleotide. The polynucleotide is not limited by length, and thus the polynucleotide can include deoxyribonucleic acid (DNA) or ribonucleic acid (RNA).

The term “transgene” refers to a polynucleotide that is introduced into a cell and is capable of being transcribed into RNA and optionally, translated and/or expressed under appropriate conditions. In aspects, it confers a desired property to a cell into which it was introduced, or otherwise leads to a desired therapeutic or diagnostic outcome.

Use of the term “about” is intended to describe values either above or below the stated value in a range of approx. +/−10%; in other forms the values may range in value either above or below the stated value in a range of approx. +/−5%.

### II. Compositions

It has been established that compositions of adeno-associated virus (AAV) vectors encoding and/or expressing all or part of a Deleted in Liver Cancer 1 (DLC1) gene can restore neuronal deficit associated with muscular atrophy in SMA patients with minimal toxicity. Compositions of AAV viruses encoding DLC1 for administration to a subject in need thereof are provided. The compositions provide gene therapy to ameliorate the symptoms of SMA. Typically, the DLC1 gene is under the control of a mammalian promoter for expression within target cells. Exemplary promoters include the SYN promoter. In some forms, the compositions of viruses include one or more additional genes for delivery into the same or a different target cell. For example, in some forms, the viruses also include one or more SMN1 genes for expression within the same or different target cell. The SMN1 gene can be under the control of the same or different promoter. In some forms, the same AAV particle includes two or more different genes for delivery to a target cell for amelioration and treatment of SMA. For example, in some forms, the same AAV particle includes one or more copies of a DLC1 gene and one or more copies of a SMN1 gene under the control of the same promoter, for delivery to the same target cell. In other forms, the composition incudes two or more populations of AAV encoding or expressing two or more different genes for expression in the same or different target cell(s). For example, in some forms, the composition includes a population of AAV encoding or expressing a DLC1 gene or DLC1 gene expression product and a population of AAV encoding or expressing a SMN1 gene, or a SMN1 gene expression product.

In some aspects, the compositions are formulated for administration to the spinal cord and/or cistema magna of a subject. In some forms the dosage of the composition includes at least 1×10¹² genome copies of a recombinant adeno-associated virus (rAAV) viral particle including a vector encoding a DLC1 gene (rAAV-DLC1). In some forms, the formulation includes at least 3.5×10¹¹ genome copies per kg body weight of (rAAV-DLC1). In further forms, the formulation includes at least 3.5×10¹² genome copies per kg body weight of (rAAV-DLC1). In some forms, the formulation includes at least 5×10¹² genome copies per kg body weight of (rAAV-DLC1). In further forms, at least 5×10¹³ genome copies per kg body weight of (rAAV-DLC1). In some forms, the formulation includes at least 2.5×10¹² genome copies. In other forms, the formulation includes at least 1.25×10¹³ genome copies. Pharmaceutical formulations of compositions of AAV encoding or expressing a DLC1 gene alone or in combination with a SMN1 gene are also provided. Typically, the formulations are suitable for administration into a subject in vivo.

### A. Deleted in Liver Cancer 1 Gene

The compositions include a Deleted in Liver Cancer 1 (DLC1) gene, and/or recombinant protein corresponding to the DLC1 gene expression product. DLC1, also known as StAR-related lipid transfer protein 12 (STARD12) is a protein which in humans is encoded by the DLC1 gene.

Human DLC1 contains 14 exons encoding an mRNA transcript of approx. 6.3 kb which produces a polypeptide of 1091 amino acids. It is located on chromosome 8 (8p21.3-22), in a region associated with genomic deletion or epigenetic silencing mechanisms that result in loss of heterozygosity in several types of solid cancers: DLC1 is frequently found to be inactivated in human hepatocellular carcinoma, nasopharyngeal, lung, breast, prostate, kidney, colon, uterine, ovarian, and gastric cancers.

The promoter region of DLC1 includes a CpG island including multiple sites which can be methylated to promote gene silencing and prevent transcription. The gene and/or gene expression product are configured for delivery into and expression/function within target cells. Typically, the DLC1 gene or expression product is or encodes one or more of five isoforms of DLC1, including DLC1-isoform 1 (DLC1-i1), DLC1-isoform 2 (DLC1-i2), DLC1-isoform 3 (DLC1-i3), DLC1-isoform 4 (DLC1-i4), or DLC1-isoform 5 (DLC1-i5).

This DLC1 gene is deleted in the primary tumor of hepatocellular carcinoma. It maps to 8p22-p21.3, a region frequently deleted in solid tumors. It is suggested that this gene is a candidate tumor suppressor gene for human liver cancer, as well as for prostate, lung, colorectal, and breast cancers.

### 1. DLC1-i1 Gene

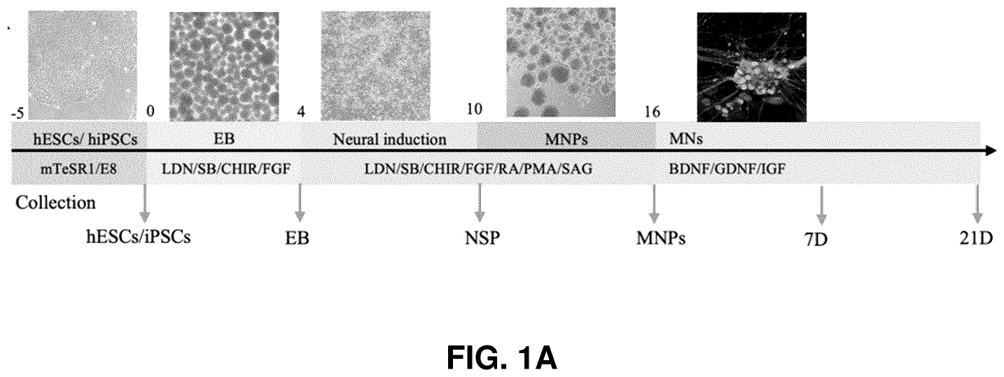

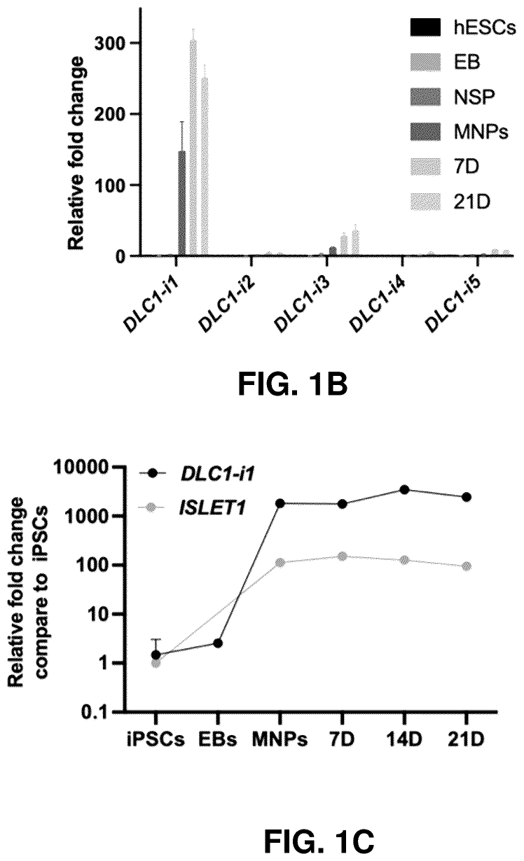

In some forms, the DLC1 gene or expression product is the DLC1-isoform 1 (DLC1-i1). It has been established that DLC1 isoform 1 (DLC1-i1) is predominantly expressed in human pluripotent stem cells-derived motor neurons (MNs) (See, FIG. 1A-C) and rat spinal cord MNs (FIG. 1D).

In some forms, the compositions include a DLC1-i1 gene, and/or a nucleic acid encoding the DLC1-i1 protein, and/or a polypeptide corresponding to the DLC1-i1 gene expression product together with (i.e., encapsidated within) a recombinant AAV virion (rAAV). For example, in an exemplary form, the rAAV includes an isolated nucleic acid (e.g., a transgene) that encodes an DLC1-i1 protein, whereby the isolated nucleic acid is packaged in any AAV viral particle described herein. Typically, the DLC1-i1 gene is an isolated nucleic acid encoding an DLC1-i1 protein from a primate such as a human. In some forms, the isolated nucleic acid encodes an DLC1-i1 protein from a primate taxonomy selected from the group consisting of a family Tarsiidae, family Callitrichidae, family Cebidae, family Aotidae, family Pitheciidae, family Atelidae, family Cercopithecidae, family Hylobatidae, and family Hominidae. In some forms, the isolated nucleic acid encodes a DLC1-i1 protein from a primate selected from a Homo sapien, a Macaca mulatta, a Pan troglodytes, a Papio anubis, a Nomascus leucogenys, a Pongo abelii, a Gorilla gorilla, a Saimiri boliviensis, and a Pan paniscus.

In some forms, the isolated nucleic acid (e.g., the transgene) encodes an SMN protein including the amino acid sequence of SEQ ID NO: 2. In some forms, the isolated nucleic acid (e.g., the transgene) includes the nucleic acid sequence selected of SEQ ID NO:1. Amino acid sequence variants of any DLC1-i1 protein provided herein are also contemplated. In some forms, the amino acid variant of a DLC1-i1 protein is a naturally occurring variant of human DLC1-i1. In some forms, the biological properties of the DLC1-i1 protein can be improved by altering the amino acid sequence encoding the protein. For example, in some forms, amino acid sequence variants of an DLC1-i1 protein are prepared by introducing appropriate modifications into the nucleic acid sequence encoding the protein or by introducing the modification by peptide synthesis. Such modifications include, for example, deletions from, insertions into, and/or substitutions within the amino acid sequence of the DLC1-i1 protein. Also contemplated herein are amino acid sequence variants of any DLC1-i1 protein that arise from natural mutations (e.g., natural selection) in the nucleic acid encoding the protein. Accordingly, provided herein are isolated nucleic acids encoding variants of a DLC1-i1 protein, wherein the isolated nucleic acid can be packaged (e.g., as a transgene) in any AAV viral particle described herein. In some forms, the isolated nucleic acid encodes an SMN protein variant including an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the amino acid sequence of any DLC1-i1 protein described herein (e.g., human DLC1-i1 protein). In some forms, the isolated nucleic acid encodes a DLC1-i1 protein variant including an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to an amino acid sequence of SEQ ID NO: 1. In some forms, the isolated nucleic acid encoding DLC1-i1 includes mutations conferring one, two, three, four, five, six, seven, eight, nine or ten amino acid substitutions while maintaining its biological function in motor neurons. In some forms, the resulting DLC1-i1 protein can express wild-type levels of activity. In some forms, the resulting DLC1-i1 protein is expressed at wild-type levels.

Isolated nucleic acid molecules encoding an DLC1-i1 protein (e.g., an DLC1-i1 protein) can be obtained by cloning or produced synthetically, or any combinations thereof. The nucleic acid can be triple-stranded, double-stranded or single-stranded, or any combination thereof. Any portion of at least one strand of the DNA can be the coding strand, also known as the sense strand, or it can be the non-coding strand, also referred to as the anti-sense strand. The isolated nucleic acids can be obtained from biological sources using any number of cloning methodologies known to those of skill in the art. The isolated nucleic acids can also be prepared by direct chemical synthesis by known methods. Nucleic acids encoding a DLC1-i1 protein can be prepared by a variety of methods known in the art including, but not limited to, isolation from a natural source or preparation by oligonucleotide mediated mutagenesis, site-directed mutagenesis, PCR mutagenesis, and cassette mutagenesis of an earlier prepared variant or a non-variant version of the DLC1-i1 protein. See Molecular Cloning: A Laboratory Manual (Sambrook et al., 4th ed., Cold Spring Harbor Laboratory Press, Cold Spring Harbor, N. Y., 2012) and Current Protocols in Molecular Biology (F. M. Ausubel, et al. eds., 2003).

An exemplary DLC1-isoform 1 (DLC1-i1) gene is described in the Genbank database as accession number: NM_182643. Therefore, in some forms, the DLC1-i1 gene has the nucleic acid sequence of:

(SEQ ID NO: 1)
AGTCTGAATTTAGTAGACAGAAGTCACTAGGAATGCCTTGACAGGATCCTGCCTTAGCT
AAGGCTCCCTCCAGCTGCAGAGGGTGTTTTTGTTAGACTCACACACTGCGTGAAACTGC
TCAGAATAGAGCCATGATCTCAACCACGAAATGGGAACTTAGATTTTGGAGAAACTAAC
GGGGACGGACTTCTTTCCTAGCCTGAGTGTTGAGCAGTGTCATGCCTTGGCGTTTCAGC
TCCTCGTTGTCTAGGTGGTGAAATGACAGAACTCATTCGCTTCTTTGATTGGTGATTTT
GAAATAATCTTTCATCAAGTTCCATCTCCTTTACCCTCATATGGAATATATCTCTCTGT
CTGTTGTTAAACTACGATGACATGTCTGTAGCTATCAGAAAGAGAAGCTGGGAAGAACA
TGTGACCCACTGGATGGGACAGCCTTTTAATTCTGATGATCGTAACACAGCATGTCATC
ATGGACTAGTAGCTGACAGCTTGCAGGCAAGTATGGAAAAAGATGCAACTCTAAATGTG
GACCGCAAAGAGAAGTGTGTTTCACTACCTGACTGCTGTCATGGATCAGAGCTGAGAGA
TTTTCCTGGGAGGCCAATGGGTCATCTTTCAAAGGATGTGGACGAAAATGACAGCCATG
AAGGTGAAGATCAGTTTCTTTCTCTGGAAGCCAGCACAGAAACACTAGTGCATGTTTCT
GATGAGGATAACAATGCTGATTTATGCCTTACAGATGATAAACAGGTTTTAAATACCCA
AGGGCAGAAAACATCAGGCCAACATATGATCCAAGGAGCAGGCTCCTTAGAAAAGGCAC
TGCCCATCATACAAAGTAACCAAGTTTCTTCTAACTCCTGGGGAATAGCTGGTGAAACT
GAATTAGCACTGGTAAAAGAAAGTGGGGAGAGAAAAGTTACTGACTCTATAAGTAAAAG
CCTGGAGCTTTGCAATGAAATAAGCTTAAGTGAAATAAAAGATGCACCCAAAGTAAATG
CAGTGGATACTTTGAACGTGAAAGATATTGCACCTGAGAAACAATTGCTTAACTCTGCT
GTAATTGCTCAGCAACGAAGGAAACCTGACCCCCCTAAAGATGAAAATGAAAGAAGCAC
CTGCAATGTAGTACAAAATGAGTTCTTGGATACTCCTTGCACAAACAGAGGACTGCCAT
TATTAAAAACAGATTTTGGAAGCTGCCTTCTGCAGCCTCCTTCCTGCCCCAATGGAATG
TCAGCTGAAAATGGCCTGGAGAAGAGTGGTTTTTCACAACATCAAAACAAAAGTCCACC
AAAGGTCAAGGCAGAAGATGGCATGCAGTGTTTACAATTAAAGGAGACCCTGGCCACCC
AGGAACCCACAGATAACCAAGTCAGACTTCGTAAGAGAAAGGAAATAAGAGAAGATCGA
GATAGGGCGCGGCTGGACTCCATGGTGCTGCTGATTATGAAACTGGACCAGCTTGATCA
GGACATAGAAAATGCCCTCAGCACCAGCTCCTCTCCATCAGGCACACCAACAAACCTGC
GGCGGCACGTTCCTGATCTGGAATCAGGATCTGAAAGTGGAGCAGATACCATTTCAGTA
AATCAGACACGAGTAAATTTGTCTTCTGACACTGAGTCCACGGACCTCCCATCTTCCAC
TCCAGTAGCCAATTCTGGAACCAAACCCAAGACTACGGCTATTCAAGGTATTTCAGAGA
AGGAAAAGGCTGAAATTGAAGCCAAGGAAGCTTGTGATTGGCTACGGGCAACTGGTTTC
CCCCAGTATGCACAGCTTTATGAAGATTTCCTGTTCCCCATCGATATTTCCTTGGTCAA
GAGAGAGCATGATTTTTTGGACAGAGATGCCATTGAGGCTCTATGCAGGCGTCTAAATA
CTTTAAACAAATGTGCGGTGATGAAGCTAGAAATTAGTCCTCATCGGAAACGAAGTGAC
GATTCAGACGAGGATGAGCCTTGTGCCATCAGTGGCAAATGGACTTTCCAAAGGGACAG
CAAGAGGTGGTCCCGGCTTGAAGAGTTTGATGTCTTTTCTCCAAAACAAGACCTGGTCC
CTGGGTCCCCAGACGACTCCCACCCGAAGGACGGCCCCAGCCCCGGAGGCACGCTGATG
GACCTCAGCGAGCGCCAGGAGGTGTCTTCCGTCCGCAGCCTCAGCAGCACTGGCAGCCT
CCCCAGCCACGCGCCCCCCAGCGAGGATGCTGCCACCCCCCGGACTAACTCCGTCATCA
GCGTTTGCTCCTCCAGCAACTTGGCAGGCAATGACGACTCTTTCGGCAGCCTGCCCTCT
CCCAAGGAACTGTCCAGCTTCAGCTTCAGCATGAAAGGCCACGAAAAAACTGCCAAGTC
CAAGACGCGCAGTCTGCTGAAACGGATGGAGAGCCTGAAGCTCAAGAGCTCCCATCACA
GCAAGCACAAAGCGCCCTCAAAGCTGGGGTTGATCATCAGCGGGCCCATCTTGCAAGAG
GGGATGGATGAGGAGAAGCTGAAGCAGCTCAACTGCGTGGAGATCTCCGCCCTCAATGG
CAACCGCATCAACGTCCCCATGGTACGAAAGAGGAGCGTTTCCAACTCCACGCAGACCA
GCAGCAGCAGCAGCCAGTCGGAGACCAGCAGCGCGGTCAGCACGCCCAGCCCTGTTACG
AGGACCCGGAGCCTCAGTGCGTGCAACAAGCGGGTGGGCATGTACTTAGAGGGCTTCGA
TCCTTTCAATCAGTCAACATTTAACAACGTGGTGGAGCAGAACTTTAAGAACCGCGAGA
GCTACCCAGAGGACACGGTGTTCTACATCCCTGAAGATCACAAGCCTGGCACTTTCCCC
AAAGCTCTCACCAATGGCAGTTTCTCCCCCTCGGGGAATAACGGCTCTGTGAACTGGAG
GACGGGAAGCTTCCACGGCCCTGGCCACATCAGCCTCAGGAGGGAAAACAGTAGCGACA
GCCCCAAGGAACTGAAGAGACGCAATTCTTCCAGCTCCATGAGCAGCCGCCTGAGCATC
TACGACAACGTGCCGGGCTCCATCCTCTACTCCAGTTCAGGGGACCTGGCGGATCTGGA
GAACGAGGACATCTTCCCCGAGCTGGACGACATCCTCTACCACGTGAAGGGGATGCAGC
GGATAGTCAATCAGTGGTCGGAGAAGTTTTCTGATGAGGGAGATTCGGACTCAGCCCTG
GACTCGGTCTCTCCCTGCCCGTCCTCTCCAAAACAGATACACCTGGATGTGGACAACGA
CCGAACCACACCCAGCGACCTGGACAGCACAGGCAACTCCCTGAATGAACCGGAAGAGC
CCTCCGAGATCCCGGAAAGAAGGGATTCTGGGGTTGGGGCTTCCCTAACCAGGTCCAAC
AGGCACCGACTGAGATGGCACAGTTTCCAGAGCTCACATCGGCCAAGCCTCAACTCTGT
ATCACTACAGATTAACTGCCAGTCTGTGGCCCAGATGAACCTGCTGCAGAAATACTCAC
TCCTAAAGCTAACGGCCCTGCTGGAGAAATACACACCTTCTAACAAGCATGGTTTTAGC
TGGGCCGTGCCCAAGTTCATGAAGAGGATCAAGGTTCCAGACTACAAGGACCGGAGTGT
GTTTGGGGTCCCACTGACGGTCAACGTGCAGCGCACAGGACAACCGTTGCCTCAGAGCA
TCCAGCAGGCCATGCGATACCTCCGGAACCATTGTTTGGATCAGGTTGGGCTCTTCAGA
AAATCGGGGGTCAAGTCCCGGATTCAGGCTCTGCGCCAGATGAATGAAGGTGCCATAGA
CTGTGTCAACTACGAAGGACAGTCTGCTTATGACGTGGCAGACATGCTGAAGCAGTATT
TTCGAGATCTTCCTGAGCCACTAATGACGAACAAACTCTCGGAAACCTTTCTACAGATC
TACCAATATGTGCCCAAGGACCAGCGCCTGCAGGCCATCAAGGCTGCCATCATGCTGCT
GCCTGACGAGAACCGGGAGGTTCTGCAGACCCTGCTTTATTTCCTGAGCGATGTCACAG
CAGCCGTAAAAGAAAACCAGATGACCCCAACCAACCTGGCCGTGTGCTTAGCGCCTTCC
CTCTTCCATCTCAACACCCTGAAGAGAGAGAATTCCTCTCCCAGGGTAATGCAAAGAAA
ACAAAGTTTGGGCAAACCAGATCAGAAAGATTTGAATGAAAACCTAGCTGCCACTCAAG
GGCTGGCCCATATGATCGCCGAGTGCAAGAAGCTTTTCCAGGTTCCCGAGGAAATGAGC
CGATGTCGTAATTCCTATACCGAACAAGAGCTGAAGCCCCTCACTCTGGAAGCACTCGG
GCACCTGGGTAATGATGACTCAGCTGACTACCAACACTTCCTCCAGGACTGTGTGGATG
GCCTGTTTAAAGAAGTCAAAGAGAAGTTTAAAGGCTGGGTCAGCTACTCCACTTCGGAG
CAGGCTGAGCTGTCCTATAAGAAGGTGAGCGAAGGACCCCCTCTGAGGCTTTGGAGGTC
AGTCATTGAAGTCCCTGCTGTGCCAGAGGAAATCTTAAAGCGCCTACTTAAAGAACAGC
ACCTCTGGGATGTAGACCTGTTGGATTCAAAAGTGATCGAAATTCTGGACAGCCAAACT
GAAATTTACCAGTATGTCCAAAACAGTATGGCACCTCATCCTGCTCGAGACTACGTTGT
TTTAAGAACCTGGAGGACTAATTTACCCAAAGGAGCCTGTGCCCTTTTACTAACCTCTG
TGGATCACGATCGCGCACCTGTGGTGGGTGTGAGGGTTAATGTGCTCTTGTCCAGGTAT
TTGATTGAACCCTGTGGGCCAGGAAAATCCAAACTCACCTACATGTGCAGAGTTGACTT
AAGGGGCCACATGCCAGAATGGTACACAAAATCTTTTGGACATTTGTGTGCAGCTGAAG
TTGTAAAGATCCGGGATTCCTTCAGTAACCAGAACACTGAAACCAAAGACACCAAATCT
AGGTGATCACTGAAGCAACGCAACCGCTTCCACCACCATGGTGTTTGTTTCTAGAACTT
TTGCCAGTCCTTGAAGAATGGGTTCTGTGTCTAATCCTGAAACAAAGAAAACTACAAGC
TGGAGTGTAGGAATTGACTATAGCAATTTGATACATTTTTAAAGCTGCTTCCTGTTTGT
TGAGGGTCTGTATTCATAGACCTTGACTGGAATATGTAAGACTGTGCAAAAAAAAAAAA
AAAATTTATGTGTATTCTTATTCAAATTGCTTCTGAGAAGCAAAACTCTTTAAATACAT
TATGGAAGATAATGAAGATACTTCATTCTCTTGTGATATCAGTGTATGCGTACCTGTGT
CGCTTTATTTGCAGTGTGTTGAGGGACTGGTGTATCCACTGGAATAGTTGGTACTCTTG
GATGTGTTTTCTCACCAAGATGAGCAAAGAAAGGTTTGCACAGAGGAGTGTGAATGTGT
GTTTGTTGCTGGCTGAATGGCAATAGATGTCTAAGGTGGATTCAGTGTCTGGCACACTG
AGACACCTCCAAGAAGGAGATTGATGCATCAGGTTCAGTTTAACCTGGAATATCTGACT
ACCCCTGAATCCACCCAGAAAGGGGGCCCAACACCCTTGTCCATTTATGGGTATTTTTT
TTCGAAGTTATTAAGCATATTCCTTTTCCACGAACCTCTTCTGTACTTTGATTGTAATA
GGTTGGCTCTTACACCCATTCCAAATGCAGTTTATTTTTAGACCCGATTGCAAATAGTG
ATGTAGTTTTAACCAGTATGGATTAGTTCAGGGATGAACTGCTCCCTCTAGCCTTACTG
GCTCTGATCCACAGGGTTTTGTTTTGTTTTGTTTTGTTTTTTGTTTAAGTCGAGATATA
AAAACTGAACACGATAACACTTACTCTTAAATCAAGCATCAACACTTTTTCCCTGTTAG
AATTCTTTGCATTTTTGTGTTTGTAACAGAAACGCCTTAAGACACTATGTTTGGGAATA
TAGGAAACTATGTGTGTCCCAAGGAAATCCCTGTAAATTTAACTCACCTACAAAAGGCT
TTTTCCCCGCCTTTGGTTGTTAACGGCATTCCTGAAAGCCACATGTGTTTATTCATTGG
GCTTGTTCTTATCAGCAAATAGGTTTTCTGGTTTTATGACTTTTTGTCTTATTTTATTT
TTCCTACATTTCTTTTTTTTTTTTTTTCCTTTAGAATGCCCTGGAAATATATTTAAGTG
GTAATGAAAAATAGTAATCATAGTAAAACGCAACAAGAAGAAAACCAACCCAAACCAGT
GAAGTTTTTTAGAACCTTTAGAAGGGTGGTCTTTATTCAGGTTTTACTGTAATGGTAAG
GATTGACTCAAGAGACAGTATTAGTAAATTTATTGTGTATGGATCAAAAGTGAATAATG
TATGAATGAGAGCTGTAAGAAGGATTTTTATTTTGTTATAATTTAGTTACCATTTTCAG
TGTTATTTCAAAGGTTCTTTGAAGAATTTTGGGGCAGGGCATCAGATTAGAGTTTTAAA
ATTTGAGTATTTTGGATATCAGTGTTCCTCATGAAGATATACATGGATATTCAATTTTG
ATGGCTTCCAGATTTGTAAGATTGTATGTTGTATATACCATTCTATTAAGAAACATGTC
CACTGTGCTTTCAAACATAGATAAAGCATGATAAAGATTATTATTTAAGATATACTTGT
ATTTATACCTCAGATATTCTTTTGGGTTTTGTACCTCAAGGCTTTTTTCTTCTTATTGT
AAATACACTTTACGTGAATACAGTCTAAGTGAAGAAAATAAATAAAAGGAAGAGGTTTA
TAACTTGCTCTATATCTGTACAGATTATAATCAATAAGTGCACTATTATTAAATGTTTA
AAGTAAGGGAAAAGTCTGGGCTGCCTTCCTTAATATTGCATCTCACTCCCACCCTTAAA
ACCACAGATTGCAAAGCATAGCATTTTAGCATCAACTACAATCAAAAGAGCGATTTGCT
GAAGGAAAAATCGGACTGCAAATCATTCCAAGGCCAAACTGCAACTGAGCCACCCACTC
CCAAACAGGAAACCCTGGTGAAGGTTCAGGAAGCACGGAGATTCTCTCCAACAAAGGTC
CAGTTAGGAAACGACGCTGAGAGGATGACGACAACGTGCAACAGCAGAAAGATGCTTGC
AAGCAGAGTCAGGGTCACCAGTGAATGCCACAAAAGTTCTCTTTCCCACTGTTTAATTT
GACAAGAGAAGAATTTGAAGGATATGAACATTTTCAAGAACTCTGCTGAGGTCACTTAG
AGCGCCATCACAACTTATTTGTGTGACTAATTGCCTAGATTGTAAGCTCTTTGAGGGCA
GGGCTTGTCTCTTACACATCTTTATAATCCCCTGCAGCGGCTTTCAGTATTTTGTACTT
GTAGGCACCTAATAAATTTATTATTTGCTATACTGAA.

Another variant of DLC1 isoform 1, is described in the Genbank database as accession number: NM_001348081 XM_017012951. Therefore, in some forms, the DLC1-i1 gene has the nucleic acid sequence of:

(SEQ ID NO: 2)
GCTCACTCATGATTATCAGACAACACATTGGCAGTGAGTTCATCTGCTCTACTCTGCTT
TGGAATAACACATGTGATTAACCAGGTGGTGAAATGACAGAACTCATTCGCTTCTTTGA
TTGGTGATTTTGAAATAATCTTTCATCAAGTTCCATCTCCTTTACCCTCATATGGAATA
TATCTCTCTGTCTGTTGTTAAACTACGATGACATGTCTGTAGCTATCAGAAAGAGAAGC
TGGGAAGAACATGTGACCCACTGGATGGGACAGCCTTTTAATTCTGATGATCGTAACAC
AGCATGTCATCATGGACTAGTAGCTGACAGCTTGCAGGCAAGTATGGAAAAAGATGCAA
CTCTAAATGTGGACCGCAAAGAGAAGTGTGTTTCACTACCTGACTGCTGTCATGGATCA
GAGCTGAGAGATTTTCCTGGGAGGCCAATGGGTCATCTTTCAAAGGATGTGGACGAAAA
TGACAGCCATGAAGGTGAAGATCAGTTTCTTTCTCTGGAAGCCAGCACAGAAACACTAG
TGCATGTTTCTGATGAGGATAACAATGCTGATTTATGCCTTACAGATGATAAACAGGTT
TTAAATACCCAAGGGCAGAAAACATCAGGCCAACATATGATCCAAGGAGCAGGCTCCTT
AGAAAAGGCACTGCCCATCATACAAAGTAACCAAGTTTCTTCTAACTCCTGGGGAATAG
CTGGTGAAACTGAATTAGCACTGGTAAAAGAAAGTGGGGAGAGAAAAGTTACTGACTCT
ATAAGTAAAAGCCTGGAGCTTTGCAATGAAATAAGCTTAAGTGAAATAAAAGATGCACC
CAAAGTAAATGCAGTGGATACTTTGAACGTGAAAGATATTGCACCTGAGAAACAATTGC
TTAACTCTGCTGTAATTGCTCAGCAACGAAGGAAACCTGACCCCCCTAAAGATGAAAAT
GAAAGAAGCACCTGCAATGTAGTACAAAATGAGTTCTTGGATACTCCTTGCACAAACAG
AGGACTGCCATTATTAAAAACAGATTTTGGAAGCTGCCTTCTGCAGCCTCCTTCCTGCC
CCAATGGAATGTCAGCTGAAAATGGCCTGGAGAAGAGTGGTTTTTCACAACATCAAAAC
AAAAGTCCACCAAAGGTCAAGGCAGAAGATGGCATGCAGTGTTTACAATTAAAGGAGAC
CCTGGCCACCCAGGAACCCACAGATAACCAAGTCAGACTTCGTAAGAGAAAGGAAATAA
GAGAAGATCGAGATAGGGCGCGGCTGGACTCCATGGTGCTGCTGATTATGAAACTGGAC
CAGCTTGATCAGGACATAGAAAATGCCCTCAGCACCAGCTCCTCTCCATCAGGCACACC
AACAAACCTGCGGCGGCACGTTCCTGATCTGGAATCAGGATCTGAAAGTGGAGCAGATA
CCATTTCAGTAAATCAGACACGAGTAAATTTGTCTTCTGACACTGAGTCCACGGACCTC
CCATCTTCCACTCCAGTAGCCAATTCTGGAACCAAACCCAAGACTACGGCTATTCAAGG
TATTTCAGAGAAGGAAAAGGCTGAAATTGAAGCCAAGGAAGCTTGTGATTGGCTACGGG
CAACTGGTTTCCCCCAGTATGCACAGCTTTATGAAGATTTCCTGTTCCCCATCGATATT
TCCTTGGTCAAGAGAGAGCATGATTTTTTGGACAGAGATGCCATTGAGGCTCTATGCAG
GCGTCTAAATACTTTAAACAAATGTGCGGTGATGAAGCTAGAAATTAGTCCTCATCGGA
AACGAAGTGACGATTCAGACGAGGATGAGCCTTGTGCCATCAGTGGCAAATGGACTTTC
CAAAGGGACAGCAAGAGGTGGTCCCGGCTTGAAGAGTTTGATGTCTTTTCTCCAAAACA
AGACCTGGTCCCTGGGTCCCCAGACGACTCCCACCCGAAGGACGGCCCCAGCCCCGGAG
GCACGCTGATGGACCTCAGCGAGCGCCAGGAGGTGTCTTCCGTCCGCAGCCTCAGCAGC
ACTGGCAGCCTCCCCAGCCACGCGCCCCCCAGCGAGGATGCTGCCACCCCCCGGACTAA
CTCCGTCATCAGCGTTTGCTCCTCCAGCAACTTGGCAGGCAATGACGACTCTTTCGGCA
GCCTGCCCTCTCCCAAGGAACTGTCCAGCTTCAGCTTCAGCATGAAAGGCCACGAAAAA
ACTGCCAAGTCCAAGACGCGCAGTCTGCTGAAACGGATGGAGAGCCTGAAGCTCAAGAG
CTCCCATCACAGCAAGCACAAAGCGCCCTCAAAGCTGGGGTTGATCATCAGCGGGCCCA
TCTTGCAAGAGGGGATGGATGAGGAGAAGCTGAAGCAGCTCAACTGCGTGGAGATCTCC
GCCCTCAATGGCAACCGCATCAACGTCCCCATGGTACGAAAGAGGAGCGTTTCCAACTC
CACGCAGACCAGCAGCAGCAGCAGCCAGTCGGAGACCAGCAGCGCGGTCAGCACGCCCA
GCCCTGTTACGAGGACCCGGAGCCTCAGTGCGTGCAACAAGCGGGTGGGCATGTACTTA
GAGGGCTTCGATCCTTTCAATCAGTCAACATTTAACAACGTGGTGGAGCAGAACTTTAA
GAACCGCGAGAGCTACCCAGAGGACACGGTGTTCTACATCCCTGAAGATCACAAGCCTG
GCACTTTCCCCAAAGCTCTCACCAATGGCAGTTTCTCCCCCTCGGGGAATAACGGCTCT
GTGAACTGGAGGACGGGAAGCTTCCACGGCCCTGGCCACATCAGCCTCAGGAGGGAAAA
CAGTAGCGACAGCCCCAAGGAACTGAAGAGACGCAATTCTTCCAGCTCCATGAGCAGCC
GCCTGAGCATCTACGACAACGTGCCGGGCTCCATCCTCTACTCCAGTTCAGGGGACCTG
GCGGATCTGGAGAACGAGGACATCTTCCCCGAGCTGGACGACATCCTCTACCACGTGAA
GGGGATGCAGCGGATAGTCAATCAGTGGTCGGAGAAGTTTTCTGATGAGGGAGATTCGG
ACTCAGCCCTGGACTCGGTCTCTCCCTGCCCGTCCTCTCCAAAACAGATACACCTGGAT
GTGGACAACGACCGAACCACACCCAGCGACCTGGACAGCACAGGCAACTCCCTGAATGA
ACCGGAAGAGCCCTCCGAGATCCCGGAAAGAAGGGATTCTGGGGTTGGGGCTTCCCTAA
CCAGGTCCAACAGGCACCGACTGAGATGGCACAGTTTCCAGAGCTCACATCGGCCAAGC
CTCAACTCTGTATCACTACAGATTAACTGCCAGTCTGTGGCCCAGATGAACCTGCTGCA
GAAATACTCACTCCTAAAGCTAACGGCCCTGCTGGAGAAATACACACCTTCTAACAAGC
ATGGTTTTAGCTGGGCCGTGCCCAAGTTCATGAAGAGGATCAAGGTTCCAGACTACAAG
GACCGGAGTGTGTTTGGGGTCCCACTGACGGTCAACGTGCAGCGCACAGGACAACCGTT
GCCTCAGAGCATCCAGCAGGCCATGCGATACCTCCGGAACCATTGTTTGGATCAGGTTG
GGCTCTTCAGAAAATCGGGGGTCAAGTCCCGGATTCAGGCTCTGCGCCAGATGAATGAA
GGTGCCATAGACTGTGTCAACTACGAAGGACAGTCTGCTTATGACGTGGCAGACATGCT
GAAGCAGTATTTTCGAGATCTTCCTGAGCCACTAATGACGAACAAACTCTCGGAAACCT
TTCTACAGATCTACCAATATGTGCCCAAGGACCAGCGCCTGCAGGCCATCAAGGCTGCC
ATCATGCTGCTGCCTGACGAGAACCGGGAGGTTCTGCAGACCCTGCTTTATTTCCTGAG
CGATGTCACAGCAGCCGTAAAAGAAAACCAGATGACCCCAACCAACCTGGCCGTGTGCT
TAGCGCCTTCCCTCTTCCATCTCAACACCCTGAAGAGAGAGAATTCCTCTCCCAGGGTA
ATGCAAAGAAAACAAAGTTTGGGCAAACCAGATCAGAAAGATTTGAATGAAAACCTAGC
TGCCACTCAAGGGCTGGCCCATATGATCGCCGAGTGCAAGAAGCTTTTCCAGGTTCCCG
AGGAAATGAGCCGATGTCGTAATTCCTATACCGAACAAGAGCTGAAGCCCCTCACTCTG
GAAGCACTCGGGCACCTGGGTAATGATGACTCAGCTGACTACCAACACTTCCTCCAGGA
CTGTGTGGATGGCCTGTTTAAAGAAGTCAAAGAGAAGTTTAAAGGCTGGGTCAGCTACT
CCACTTCGGAGCAGGCTGAGCTGTCCTATAAGAAGGTGAGCGAAGGACCCCCTCTGAGG
CTTTGGAGGTCAGTCATTGAAGTCCCTGCTGTGCCAGAGGAAATCTTAAAGCGCCTACT
TAAAGAACAGCACCTCTGGGATGTAGACCTGTTGGATTCAAAAGTGATCGAAATTCTGG
ACAGCCAAACTGAAATTTACCAGTATGTCCAAAACAGTATGGCACCTCATCCTGCTCGA
GACTACGTTGTTTTAAGAACCTGGAGGACTAATTTACCCAAAGGAGCCTGTGCCCTTTT
ACTAACCTCTGTGGATCACGATCGCGCACCTGTGGTGGGTGTGAGGGTTAATGTGCTCT
TGTCCAGGTATTTGATTGAACCCTGTGGGCCAGGAAAATCCAAACTCACCTACATGTGC
AGAGTTGACTTAAGGGGCCACATGCCAGAATGGTACACAAAATCTTTTGGACATTTGTG
TGCAGCTGAAGTTGTAAAGATCCGGGATTCCTTCAGTAACCAGAACACTGAAACCAAAG
ACACCAAATCTAGGTGATCACTGAAGCAACGCAACCGCTTCCACCACCATGGTGTTTGT
TTCTAGAACTTTTGCCAGTCCTTGAAGAATGGGTTCTGTGTCTAATCCTGAAACAAAGA
AAACTACAAGCTGGAGTGTAGGAATTGACTATAGCAATTTGATACATTTTTAAAGCTGC
TTCCTGTTTGTTGAGGGTCTGTATTCATAGACCTTGACTGGAATATGTAAGACTGTGCA
AAAAAAAAAAAAAAATTTATGTGTATTCTTATTCAAATTGCTTCTGAGAAGCAAAACTC
TTTAAATACATTATGGAAGATAATGAAGATACTTCATTCTCTTGTGATATCAGTGTATG
CGTACCTGTGTCGCTTTATTTGCAGTGTGTTGAGGGACTGGTGTATCCACTGGAATAGT
TGGTACTCTTGGATGTGTTTTCTCACCAAGATGAGCAAAGAAAGGTTTGCACAGAGGAG
TGTGAATGTGTGTTTGTTGCTGGCTGAATGGCAATAGATGTCTAAGGTGGATTCAGTGT
CTGGCACACTGAGACACCTCCAAGAAGGAGATTGATGCATCAGGTTCAGTTTAACCTGG
AATATCTGACTACCCCTGAATCCACCCAGAAAGGGGGCCCAACACCCTTGTCCATTTAT
GGGTATTTTTTTTCGAAGTTATTAAGCATATTCCTTTTCCACGAACCTCTTCTGTACTT
TGATTGTAATAGGTTGGCTCTTACACCCATTCCAAATGCAGTTTATTTTTAGACCCGAT
TGCAAATAGTGATGTAGTTTTAACCAGTATGGATTAGTTCAGGGATGAACTGCTCCCTC
TAGCCTTACTGGCTCTGATCCACAGGGTTTTGTTTTGTTTTGTTTTGTTTTTTGTTTAA
GTCGAGATATAAAAACTGAACACGATAACACTTACTCTTAAATCAAGCATCAACACTTT
TTCCCTGTTAGAATTCTTTGCATTTTTGTGTTTGTAACAGAAACGCCTTAAGACACTAT
GTTTGGGAATATAGGAAACTATGTGTGTCCCAAGGAAATCCCTGTAAATTTAACTCACC
TACAAAAGGCTTTTTCCCCGCCTTTGGTTGTTAACGGCATTCCTGAAAGCCACATGTGT
TTATTCATTGGGCTTGTTCTTATCAGCAAATAGGTTTTCTGGTTTTATGACTTTTTGTC
TTATTTTATTTTTCCTACATTTCTTTTTTTTTTTTTTTCCTTTAGAATGCCCTGGAAAT
ATATTTAAGTGGTAATGAAAAATAGTAATCATAGTAAAACGCAACAAGAAGAAAACCAA
CCCAAACCAGTGAAGTTTTTTAGAACCTTTAGAAGGGTGGTCTTTATTCAGGTTTTACT
GTAATGGTAAGGATTGACTCAAGAGACAGTATTAGTAAATTTATTGTGTATGGATCAAA
AGTGAATAATGTATGAATGAGAGCTGTAAGAAGGATTTTTATTTTGTTATAATTTAGTT
ACCATTTTCAGTGTTATTTCAAAGGTTCTTTGAAGAATTTTGGGGCAGGGCATCAGATT
AGAGTTTTAAAATTTGAGTATTTTGGATATCAGTGTTCCTCATGAAGATATACATGGAT
ATTCAATTTTGATGGCTTCCAGATTTGTAAGATTGTATGTTGTATATACCATTCTATTA
AGAAACATGTCCACTGTGCTTTCAAACATAGATAAAGCATGATAAAGATTATTATTTAA
GATATACTTGTATTTATACCTCAGATATTCTTTTGGGTTTTGTACCTCAAGGCTTTTTT
CTTCTTATTGTAAATACACTTTACGTGAATACAGTCTAAGTGAAGAAAATAAATAAAAG
GAAGAGGTTTATAACTTGCTCTATATCTGTACAGATTATAATCAATAAGTGCACTATTA
TTAAATGTTTAAAGTAAGGGAAAAGTCTGGGCTGCCTTCCTTAATATTGCATCTCACTC
CCACCCTTAAAACCACAGATTGCAAAGCATAGCATTTTAGCATCAACTACAATCAAAAG
AGCGATTTGCTGAAGGAAAAATCGGACTGCAAATCATTCCAAGGCCAAACTGCAACTGA
GCCACCCACTCCCAAACAGGAAACCCTGGTGAAGGTTCAGGAAGCACGGAGATTCTCTC
CAACAAAGGTCCAGTTAGGAAACGACGCTGAGAGGATGACGACAACGTGCAACAGCAGA
AAGATGCTTGCAAGCAGAGTCAGGGTCACCAGTGAATGCCACAAAAGTTCTCTTTCCCA
CTGTTTAATTTGACAAGAGAAGAATTTGAAGGATATGAACATTTTCAAGAACTCTGCTG
AGGTCACTTAGAGCGCCATCACAACTTATTTGTGTGACTAATTGCCTAGATTGTAAGCT
CTTTGAGGGCAGGGCTTGTCTCTTACACATCTTTATAATCCCCTGCAGCGGCTTTCAGT
ATTTTGTACTTGTAGGCACCTAATAAATTTATTATTTGCTATACTGAA.

The DLC1-i1 gene coded protein is described in the Genbank database with accession number: NP_872584; NP_001335010; XP_016868440. Therefore, in some forms, the DLC1-i1 gene coded protein has the amino acid sequence of:

(SEQ ID NO: 3)
MSVAIRKRSWEEHVTHWMGQPFNSDDRNTACHHGLVADSLQASMEKDATLNVDRKEKCV
SLPDCCHGSELRDFPGRPMGHLSKDVDENDSHEGEDQFLSLEASTETLVHVSDEDNNAD
LCLTDDKQVLNTQGQKTSGQHMIQGAGSLEKALPIIQSNQVSSNSWGIAGETELALVKE
SGERKVTDSISKSLELCNEISLSEIKDAPKVNAVDTLNVKDIAPEKQLLNSAVIAQQRR
KPDPPKDENERSTCNVVQNEFLDTPCTNRGLPLLKTDFGSCLLQPPSCPNGMSAENGLE
KSGFSQHQNKSPPKVKAEDGMQCLQLKETLATQEPTDNQVRLRKRKEIREDRDRARLDS
MVLLIMKLDQLDQDIENALSTSSSPSGTPTNLRRHVPDLESGSESGADTISVNQTRVNL
SSDTESTDLPSSTPVANSGTKPKTTAIQGISEKEKAEIEAKEACDWLRATGFPQYAQLY
EDFLFPIDISLVKREHDFLDRDAIEALCRRLNTLNKCAVMKLEISPHRKRSDDSDEDEP
CAISGKWTFQRDSKRWSRLEEFDVFSPKQDLVPGSPDDSHPKDGPSPGGTLMDLSERQE
VSSVRSLSSTGSLPSHAPPSEDAATPRINSVISVCSSSNLAGNDDSFGSLPSPKELSSF
SFSMKGHEKTAKSKTRSLLKRMESLKLKSSHHSKHKAPSKLGLIISGPILQEGMDEEKL
KQLNCVEISALNGNRINVPMVRKRSVSNSTQTSSSSSQSETSSAVSTPSPVTRTRSLSA
CNKRVGMYLEGFDPFNQSTENNVVEQNFKNRESYPEDTVFYIPEDHKPGTFPKALTNGS
FSPSGNNGSVNWRTGSFHGPGHISLRRENSSDSPKELKRRNSSSSMSSRLSIYDNVPGS
ILYSSSGDLADLENEDIFPELDDILYHVKGMQRIVNQWSEKFSDEGDSDSALDSVSPCP
SSPKQIHLDVDNDRTTPSDLDSTGNSLNEPEEPSEIPERRDSGVGASLTRSNRHRLRWH
SFQSSHRPSLNSVSLQINCQSVAQMNLLQKYSLLKLTALLEKYTPSNKHGFSWAVPKEM
KRIKVPDYKDRSVFGVPLTVNVQRTGQPLPQSIQQAMRYLRNHCLDQVGLFRKSGVKSR
IQALRQMNEGAIDCVNYEGQSAYDVADMLKQYFRDLPEPLMINKLSETFLQIYQYVPKD
QRLQAIKAAIMLLPDENREVLQTLLYFLSDVTAAVKENQMTPTNLAVCLAPSLFHLNTL
KRENSSPRVMQRKQSLGKPDQKDLNENLAATQGLAHMIAECKKLFQVPEEMSRCRNSYT
EQELKPLTLEALGHLGNDDSADYQHFLQDCVDGLFKEVKEKFKGWVSYSTSEQAELSYK
KVSEGPPLRLWRSVIEVPAVPEEILKRLLKEQHLWDVDLLDSKVIEILDSQTEIYQYVQ
NSMAPHPARDYVVLRTWRTNLPKGACALLLTSVDHDRAPVVGVRVNVLLSRYLIEPCGP
GKSKLTYMCRVDLRGHMPEWYTKSFGHLCAAEVVKIRDSFSNQNTETKDTKSR.

### 2. SMN1 Gene

In some forms, the compositions also include a survival motor neuron 1 (SMN1) gene, and/or or polypeptide corresponding to the SMN1 gene expression product in addition to a Deleted in Liver Cancer 1 (DLC1) gene, and/or polypeptide corresponding to the DLC1 gene expression product.

Therefore, compositions including the SMN1 gene for expression in a cell are provided. The compositions include viral vectors for expression of SMN1 genes and gene expression products alone, as well as including both the DLC1 gene and gene expression products together with the SMN1 genes and gene expression products.

In some forms, the rAAV includes an isolated nucleic acid (e.g., a transgene) that encodes an SMN protein, wherein the isolated nucleic acid can be packaged in any AAV viral particle described herein. Typically, the SMN gene is an isolated nucleic acid encoding an SMN protein from a primate such as a human. In some forms, the isolated nucleic acid encodes an SMN protein from a primate taxonomy selected from the group consisting of a family Tarsiidae, family Callitrichidae, family Cebidae, family Aotidae, family Pitheciidae, family Atelidae, family Cercopithecidae, family Hylobatidae, and family Hominidae. In some forms, the isolated nucleic acid encodes an SMN protein from a primate selected from a Homo sapien, a Macaca mulatta, a Pan troglodytes, a Papio anubis, a Nomascus leucogenys, a Pongo abelii, a Gorilla gorilla, a Saimiri boliviensis, and a Pan paniscus.

In some forms, the isolated nucleic acid encodes an SMN1 mRNA identified by a NCBI Reference Sequence (RefSeq) number selected from NM_000344.3, NM_022874.2, NM_001260664.1, and NM_001131470.2. In some form, the isolated nucleic acid encodes an SMN protein identified by a NCBI RefSeq number selected from NP 000335.1, NP 075012.1, NP 001247593.1, XP 001156488.1, XP 001156435.1, XP 001156259.1, XP 001156201.1, XP 003266089.1, XP 003266087.1, XP 003266086.1, XP 003266090.1, NP 001124942.1, XP 004058779.1, XP 003925817.1, XP 003925818.1, XP 003925819.1, XP 003806815.1, XP 003806816.1, XP 003806817.1, and XP 003806818.1. In some forms, the isolated nucleic acid (e.g., the transgene) encodes an SMN protein including the amino acid sequence of SEQ ID NO:4. In some forms, the isolated nucleic acid (e.g., the transgene) includes the nucleic acid sequence of SEQ ID NOs:3. Amino acid sequence variants of any SMN protein provided herein are also contemplated. In some forms, the amino acid variant of an SMN protein is a naturally occurring variant of SMN. In some forms, the biological properties of the SMN protein can be improved by altering the amino acid sequence encoding the protein. For example, in some forms, amino acid sequence variants of an SMN protein are prepared by introducing appropriate modifications into the nucleic acid sequence encoding the protein or by introducing the modification by peptide synthesis. Such modifications include, for example, deletions from, insertions into, and/or substitutions within the amino acid sequence of the SMN protein. Also contemplated herein are amino acid sequence variants of any SMN protein that arise from natural mutations (e.g., natural selection) in the nucleic acid encoding the protein. Accordingly, provided herein are isolated nucleic acids encoding variants of an SMN protein, wherein the isolated nucleic acid can be packaged (e.g., as a transgene) in any AAV viral particle described herein. In some forms, the isolated nucleic acid encodes an SMN protein variant including an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the amino acid sequence of any SMN protein described herein (e.g., human SMN protein). In some forms, the isolated nucleic acid encodes an SMN protein variant including an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to an amino acid sequence of SEQ ID NO:4. In some forms, the isolated nucleic acid encoding SMN includes mutations conferring one, two, three, four, five, six, seven, eight, nine or ten amino acid substitutions while maintaining its biological function in motor neurons. In some forms, the resulting SMN protein can express wild-type levels of activity. In some forms, the resulting SMN protein is expressed at wild-type levels.

Isolated nucleic acid molecules encoding an SMN protein (e.g., an SMN protein) can be obtained by cloning or produced synthetically, or any combinations thereof. The nucleic acid can be triple-stranded, double-stranded or single-stranded, or any combination thereof. Any portion of at least one strand of the DNA can be the coding strand, also known as the sense strand, or it can be the non-coding strand, also referred to as the anti-sense strand. The isolated nucleic acids can be obtained from biological sources using any number of cloning methodologies known to those of skill in the art. The isolated nucleic acids can also be prepared by direct chemical synthesis by known methods. Nucleic acids encoding an SMN protein can be prepared by a variety of methods known in the art including, but not limited to, isolation from a natural source or preparation by oligonucleotide mediated mutagenesis, site-directed mutagenesis, PCR mutagenesis, and cassette mutagenesis of an earlier prepared variant or a non-variant version of the SMN protein.

In some forms, the nucleic acid encoding the SMN1 protein has the nucleic acid sequence of:

(SEQ ID NO: 4)
ATGGCGATGAGCAGCGGCGGCAGTGGTGGCGGCGTCCCGGAGCAGGAGGATTCCGTGCT
GTTCCGGCGCGGCACAGGCCAGAGCGATGATTCTGACATTTGGGATGATACAGCACTGA
TAAAAGCATATGATAAAGCTGTGGCTTCATTTAAGCATGCTCTAAAGAATGGTGACATT
TGTGAAACTTCGGGTAAACCAAAAACCACACCTAAAAGAAAACCTGCTAAGAAGAATAA
AAGCCAAAAGAAGAATACTGCAGCTTCCTTACAACAGTGGAAAGTTGGGGACAAATGTT
CTGCCATTTGGTCAGAAGACGGTTGCATTTACCCAGCTACCATTGCTTCAATTGATTTT
AAGAGAGAAACCTGTGTTGTGGTTTACACTGGATATGGAAATAGAGAGGAGCAAAATCT
GTCCGATCTACTTTCCCCAATCTGTGAAGTAGCTAATAATATAGAACAGAATGCTCAAG
AGAATGAAAATGAAAGCCAAGTTTCAACAGATGAAAGTGAGAACTCCAGGTCTCCTGGA
AATAAATCAGATAACATCAAGCCCAAATCTGCTCCATGGAACTCTTTTCTCCCTCCACC
ACCCCCCATGCCAGGGCCAAGACTGGGACCAGGAAAGCCAGGTCTAAAATTCAATGGCC
CACCACCGCCACCGCCACCACCACCACCCCACTTACTATCATGCTGGCTGCCTCCATTT
CCTTCTGGACCACCAATAATTCCCCCACCACCTCCCATATGTCCAGATTCTCTTGATGA
TGCTGATGCTTTGGGAAGTATGTTAATTTCATGGTACATGAGTGGCTATCATACTGGCT
ATTATATGGGTTTCAGACAAAATCAAAAAGAAGGAAGGTGCTCACATTCCTTAAATTAA.

In some forms, the SMN-1 gene produces a protein having the amino acid sequence of:

(SEQ ID NO: 5)
MAMSSGGSGGGVPEQEDSVLFRRGTGQSDDSDIWDDTALIKAYDKAVASFKHALKNGDI
CETSGKPKTTPKRKPAKKNKSQKKNTAASLQQWKVGDKCSAIWSEDGCIYPATIASIDE
KRETCVVVYTGYGNREEQNLSDLLSPICEVANNIEQNAQENENESQVSTDESENSRSPG
NKSDNIKPKSAPWNSFLPPPPPMPGPRLGPGKPGLKFNGPPPPPPPPPPHLLSCWLPPF
PSGPPIIPPPPPICPDSLDDADALGSMLISWYMSGYHTGYYMGFRQNQKEGRCSHSLN.

### B. Adeno-Associated Virus (AAV) Vector

A preferred vector for delivering the DLC1 gene 1 is an adeno-associated viral (AAV) vector.

AAV is a non-pathogenic, single-stranded DNA virus that has been actively employed for delivering therapeutic genes in both in vitro and in vivo systems (Choi, et al., Curr. Gene Ther., 5:299-310, (2005)). AAV is a replication-deficient virus that belongs to the parvovirus family, and is dependent on co-infection with other viruses, mainly adenoviruses, to replicate. Initially distinguished serologically, molecular cloning of AAV genes has identified hundreds of unique AAV strains in numerous species. Each end of the single-stranded DNA genome contains an inverted terminal repeat (ITR), which is the only cis-acting element required for genome replication and packaging. The single-stranded AAV genome contains three genes, Rep (Replication), Cap (Capsid), and aap (Assembly). These three genes give rise to at least nine gene products through the use of three promoters, alternative translation start sites, and differential splicing. These coding sequences are flanked by the ITRs. The Rep gene encodes four proteins (Rep78, Rep68, Rep52, and Rep40), while Cap expression gives rise to the viral capsid proteins (VP; VP1/VP2/VP3), which form the outer capsid shell that protects the viral genome, as well as being actively involved in cell binding and internalization. It is estimated that the viral coat includes 60 proteins, arranged into an icosahedral structure with the capsid proteins in a molar ratio of 1:1:10 (VP1:VP2:VP3).

Recombinant AAV (rAAV), which lacks viral DNA, is essentially a protein-based nanoparticle engineered to traverse the cell membrane, where it can ultimately traffic and deliver its DNA cargo into the nucleus of a cell. In the absence of Rep proteins, ITR-flanked transgenes encoded within rAAV can form circular concatemers that persist as episomes in the nucleus of transduced cells. Because recombinant episomal DNA does not integrate into host genomes, it will eventually be diluted over time as the cell undergoes repeated rounds of replication. This will eventually result in the loss of the transgene and transgene expression, with the rate of transgene loss dependent on the turnover rate of the transduced cell. These characteristics make rAAV ideal for certain gene therapy applications.

AAV can be advantageous over other viral vectors due to low toxicity (this can be due to the purification method not requiring ultra centrifugation of cell particles that can activate the immune response) and low probability of causing insertional mutagenesis because AAV does not integrate into the host genome (primarily remaining episomal).

In some forms, the AAV vector used in the disclosed compositions and methods is a naturally occurring serotype of AAV including, but not limited to, AAV1, AAV2, AAV3, AAV4, AAV5, AAV6, AAV7, AAV8, AAV9, AAV10, AAV11, AAV12, artificial variants such as AAV.rh10, AAV.rh32/33, AAV.rh43, AAV.rh64R1, rAAV2-retro, AAV-DJ, AAV-PHP.B, AAV-PHP.S, AAV-PHP.eB, or other engineered versions of AAV. In preferred forms, the AAV used in the disclosed compositions and methods is AAV6 or AAV9.

Twelve natural serotypes of AAV have thus far been identified, with the best characterized and most commonly used being AAV2. These serotypes differ in their tropism, or the types of cells they infect, making AAV a very useful system for preferentially transducing specific cell types. For example, AAV serotypes 1, 2, 5 or a hybrid capsid AAV1, AAV2, AAV5 or any combination thereof can be used for targeting brain or neuronal cells; AAV4 can be selected for targeting cardiac cells. AAV8 is useful for delivery to the liver cells. Researchers have further refined the tropism of AAV through pseudotyping, or the mixing of a capsid and genome from different viral serotypes. These serotypes are denoted using a slash, so that AAV2/5 indicates a virus containing the genome of serotype 2 packaged in the capsid from serotype 5. Use of these pseudotyped viruses can improve transduction efficiency, as well as alter tropism. For example, AAV2/5 targets neurons that are not efficiently transduced by AAV2/2, and is distributed more widely in the brain, indicating improved transduction efficiency.

One of skill in the art would be able to determine the optimal AAV serotype to be used for the respective application. The AAV can be AAV1, AAV2, AAV3, AAV4, AAV5, AAV6, AAV7, AAV8, AAV9, AAV10, AAV11, AAV12, artificial variants such as AAV.rh10, AAV.rh32/33, AAV.rh43, AAV.rh64R1, rAAV2-retro, AAV-DJ, AAV-PHP.B, AAV-PHP.S, and AAV-PHP.eB, or combinations thereof. In preferred forms, the AAV vector is AAV6 or AAV9.

In some forms, the rAAV viral particle includes an AAV serotype capsid from Clades A-F.

1. Recombinant AAV Vector (rAAV)

Typically, the vector is a recombinant AAV vector (rAAV vector), including a polynucleotide vector having one or more heterologous sequences (i.e., nucleic acid sequence not of AAV origin) that are flanked by at least one, preferably two, AAV inverted terminal repeat sequences (ITRs). Such rAAV vectors can be replicated and packaged into infectious viral particles when present in a host cell that has been infected with a suitable helper virus (or that is expressing suitable helper functions) and that is expressing AAV rep and cap gene products (i.e. AAV Rep and Cap proteins). When a rAAV vector is incorporated into a larger polynucleotide (e.g., in a chromosome or in another vector such as a plasmid used for cloning or transfection), then the rAAV vector may be referred to as a “pro-vector”, which can be “rescued” by replication and encapsidation in the presence of AAV packaging functions and suitable helper functions. An rAAV vector can be in any of a number of forms, including, but not limited to, plasmids, linear artificial chromosomes, complexed with lipids, encapsulated within liposomes, and, most preferable, encapsidated in a viral particle, particularly an AAV particle. In some forms, the rAAV vector is packaged into an AAV virus capsid to generate a “recombinant adeno-associated viral particle (rAAV particle)”. The rAAV includes a vector encoding the gene(s) for delivery to a target cell(s). In some forms, the vector includes AAV1, AAV2, AAV3, AAV4, AAV5, AAV6, AAV7, AAVS, AAVrh8, AAV9, AAV10, AAVrh10, AAV11, or AAV12 serotype inverted terminal repeats (ITRs).

An “inverted terminal repeat” or “ITR” sequence is a term well understood in the art and refers to relatively short sequences found at the termini of viral genomes which are in opposite orientation. An exemplary AAV inverted terminal repeat (ITR) sequence, a term well-understood in the art, is an approximately 145 nucleotide sequence that is present at both termini of the native single-stranded AAV genome. The outermost 125 nucleotides of the ITR can be present in either of two alternative orientations, leading to heterogeneity between different AAV genomes and between the two ends of a single AAV genome. The outermost 125 nucleotides also contains several shorter regions of self-complementarity (designated A, A′, B, B′, C, C′ and D regions), allowing intra strand base-pairing to occur within this portion of the ITR.

In some forms, the vector includes AAV serotype 2 ITRs. In other forms, the rAAV viral particle includes an AAV serotype capsid from Clades A-F. In further forms, the ITR and the capsid are derived from the same AAV serotype. In other forms, the ITR and the capsid are derived from different AAV serotypes. In some forms, the rAAV viral particle includes an AAV-9 capsid, and the vector includes AAV2 ITRs.

An exemplary nucleic acid sequence for a vector for use with the AAV system is:

(SEQ ID NO: 6)
CCTGCAGGCAGCTGCGCGCTCGCTCGCTCACTGAGGCCGCCCGGGCAAAGCCCGGGCGT
CGGGCGACCTTTGGTCGCCCGGCCTCAGTGAGCGAGCGAGCGCGCAGAGAGGGAGTGGC
CAACTCCATCACTAGGGGTTCCTGCGGCCGCACGCGTTCTAGACTATACAGTTGAAGTC
GGAAGTTTACATACACTTAAGTTGGAGTCATTAAAACTCGTTTTTCAACTACTCCACAA
ATTTCTTGTTAACAAACAATAGTTTTGGCAAGTCAGTTAGGACATCTACTTTGTGCATG
ACACAAGTCATTTTTCCAACAATTGTTTACAGACAGATTATTTCACTTATAATTCACTG
TATCACAATTCCAGTGGGTCAGAAGTTTACATACACTAAGTTGACTGTGCCTTTAAACA
GCTTGGAAAATTCCAGAAAATGATGTCATGGCTTTAGAGAGGATCCTAGGTCTTGAAAG
GAGTGGGAATTGGCTCCGGTGCCCGTCAGTGGGCAGAGCGCACATCGCCCACAGTCCCC
GAGAAGTTGGGGGGAGGGGTCGGCAATTGATCCGGTGCCTAGAGAAGGTGGCGCGGGGT
AAACTGGGAAAGTGATGTCGTGTACTGGCTCCGCCTTTTTCCCGAGGGTGGGGGAGAAC
CGTATATAAGTGCAGTAGTCGCCGTGAACGTTCTTTTTCGCAACGGGTTTGCCGCCAGA
ACACAGGACCGGTTCTAGACGTACGGCCACCATGGGAATTCTTGAGTGTATGTAAACTT
CTGACCCACTGGGAATGTGATGAAAGAAATAAAAGCTGAAATGAATCATTCTCTCTACT
ATTATTCTGATATTTCACATTCTTAAAATAAAGTGGTGATCCTAACTGACCTAAGACAG
GGAATTTTTACTAGGATTAAATGTCAGGAATTGTGAAAAAGTGAGTTTAAATGTATTTG
GCTAAGGTGTATGTAAACTTCCGACTTCAACTGTATAGGCATGCGGTAACCACGTGCGG
ACCGAGCGGCCGCAGGAACCCCTAGTGATGGAGTTGGCCACTCCCTCTCTGCGCGCTCG
CTCGCTCACTGAGGCCGGGCGACCAAAGGTCGCCCGACGCCCGGGCTTTGCCCGGGCGG
CCTCAGTGAGCGAGCGAGCGCGCAGCTGCCTGCAGGGGCGCCTGATGCGGTATTTTCTC
CTTACGCATCTGTGCGGTATTTCACACCGCATACGTCAAAGCAACCATAGTACGCGCCC
TGTAGCGGCGCATTAAGCGCGGCGGGTGTGGTGGTTACGCGCAGCGTGACCGCTACACT
TGCCAGCGCCCTAGCGCCCGCTCCTTTCGCTTTCTTCCCTTCCTTTCTCGCCACGTTCG
CCGGCTTTCCCCGTCAAGCTCTAAATCGGGGGCTCCCTTTAGGGTTCCGATTTAGTGCT
TTACGGCACCTCGACCCCAAAAAACTTGATTTGGGTGATGGTTCACGTAGTGGGCCATC
GCCCTGATAGACGGTTTTTCGCCCTTTGACGTTGGAGTCCACGTTCTTTAATAGTGGAC
TCTTGTTCCAAACTGGAACAACACTCAACCCTATCTCGGGCTATTCTTTTGATTTATAA
GGGATTTTGCCGATTTCGGCCTATTGGTTAAAAAATGAGCTGATTTAACAAAAATTTAA
CGCGAATTTTAACAAAATATTAACGTTTACAATTTTATGGTGCACTCTCAGTACAATCT
GCTCTGATGCCGCATAGTTAAGCCAGCCCCGACACCCGCCAACACCCGCTGACGCGCCC
TGACGGGCTTGTCTGCTCCCGGCATCCGCTTACAGACAAGCTGTGACCGTCTCCGGGAG
CTGCATGTGTCAGAGGTTTTCACCGTCATCACCGAAACGCGCGAGACGAAAGGGCCTCG
TGATACGCCTATTTTTATAGGTTAATGTCATGATAATAATGGTTTCTTAGACGTCAGGT
GGCACTTTTCGGGGAAATGTGCGCGGAACCCCTATTTGTTTATTTTTCTAAATACATTC
AAATATGTATCCGCTCATGAGACAATAACCCTGATAAATGCTTCAATAATATTGAAAAA
GGAAGAGTATGAGTATTCAACATTTCCGTGTCGCCCTTATTCCCTTTTTTGCGGCATTT
TGCCTTCCTGTTTTTGCTCACCCAGAAACGCTGGTGAAAGTAAAAGATGCTGAAGATCA
GTTGGGTGCACGAGTGGGTTACATCGAACTGGATCTCAACAGCGGTAAGATCCTTGAGA
GTTTTCGCCCCGAAGAACGTTTTCCAATGATGAGCACTTTTAAAGTTCTGCTATGTGGC
GCGGTATTATCCCGTATTGACGCCGGGCAAGAGCAACTCGGTCGCCGCATACACTATTC
TCAGAATGACTTGGTTGAGTACTCACCAGTCACAGAAAAGCATCTTACGGATGGCATGA
CAGTAAGAGAATTATGCAGTGCTGCCATAACCATGAGTGATAACACTGCGGCCAACTTA
CTTCTGACAACGATCGGAGGACCGAAGGAGCTAACCGCTTTTTTGCACAACATGGGGGA
TCATGTAACTCGCCTTGATCGTTGGGAACCGGAGCTGAATGAAGCCATACCAAACGACG
AGCGTGACACCACGATGCCTGTAGCAATGGCAACAACGTTGCGCAAACTATTAACTGGC
GAACTACTTACTCTAGCTTCCCGGCAACAATTAATAGACTGGATGGAGGCGGATAAAGT
TGCAGGACCACTTCTGCGCTCGGCCCTTCCGGCTGGCTGGTTTATTGCTGATAAATCTG
GAGCCGGTGAGCGTGGGTCTCGCGGTATCATTGCAGCACTGGGGCCAGATGGTAAGCCC
TCCCGTATCGTAGTTATCTACACGACGGGGAGTCAGGCAACTATGGATGAACGAAATAG
ACAGATCGCTGAGATAGGTGCCTCACTGATTAAGCATTGGTAACTGTCAGACCAAGTTT
ACTCATATATACTTTAGATTGATTTAAAACTTCATTTTTAATTTAAAAGGATCTAGGTG
AAGATCCTTTTTGATAATCTCATGACCAAAATCCCTTAACGTGAGTTTTCGTTCCACTG
AGCGTCAGACCCCGTAGAAAAGATCAAAGGATCTTCTTGAGATCCTTTTTTTCTGCGCG
TAATCTGCTGCTTGCAAACAAAAAAACCACCGCTACCAGCGGTGGTTTGTTTGCCGGAT
CAAGAGCTACCAACTCTTTTTCCGAAGGTAACTGGCTTCAGCAGAGCGCAGATACCAAA
TACTGTCCTTCTAGTGTAGCCGTAGTTAGGCCACCACTTCAAGAACTCTGTAGCACCGC
CTACATACCTCGCTCTGCTAATCCTGTTACCAGTGGCTGCTGCCAGTGGCGATAAGTCG
TGTCTTACCGGGTTGGACTCAAGACGATAGTTACCGGATAAGGCGCAGCGGTCGGGCTG
AACGGGGGGTTCGTGCACACAGCCCAGCTTGGAGCGAACGACCTACACCGAACTGAGAT
ACCTACAGCGTGAGCTATGAGAAAGCGCCACGCTTCCCGAAGGGAGAAAGGCGGACAGG
TATCCGGTAAGCGGCAGGGTCGGAACAGGAGAGCGCACGAGGGAGCTTCCAGGGGGAAA
CGCCTGGTATCTTTATAGTCCTGTCGGGTTTCGCCACCTCTGACTTGAGCGTCGATTTT
TGTGATGCTCGTCAGGGGGGCGGAGCCTATGGAAAAACGCCAGCAACGCGGCCTTTTTA
CGGTTCCTGGCCTTTTGCTGGCCTTTTGCTCACATGT.

In some forms, the vector is a pAAV-hSYN-EGFP scaffold vector, including a SYN promoter for expression of an encoded gene at high levels. An exemplary scaffold vector is pAAV-hSyn-EGFP having 5265 base pairs (bp) (commercially available from Addgene, catalogue number: 504465). Therefore, in some forms, a DLC-i1 gene and/or a SMN1 gene is delivered by recombination into a vector having a nucleic acid sequence of:

(SEQ ID NO: 7)
CATGTCCTGCAGGCAGCTGCGCGCTCGCTCGCTCACTGAGGCCGCCCGGGCGTCGGGCG
ACCTTTGGTCGCCCGGCCTCAGTGAGCGAGCGAGCGCGCAGAGAGGGAGTGGCCAACTC
CATCACTAGGGGTTCCTGCGGCCGCACGCGTGTGTCTAGACTGCAGAGGGCCCTGCGTA
TGAGTGCAAGTGGGTTTTAGGACCAGGATGAGGCGGGGTGGGGGTGCCTACCTGACGAC
CGACCCCGACCCACTGGACAAGCACCCAACCCCCATTCCCCAAATTGCGCATCCCCTAT
CAGAGAGGGGGAGGGGAAACAGGATGCGGCGAGGCGCGTGCGCACTGCCAGCTTCAGCA
CCGCGGACAGTGCCTTCGCCCCCGCCTGGCGGCGCGCGCCACCGCCGCCTCAGCACTGA
AGGCGCGCTGACGTCACTCGCCGGTCCCCCGCAAACTCCCCTTCCCGGCCACCTTGGTC
GCGTCCGCGCCGCCGCCGGCCCAGCCGGACCGCACCACGCGAGGCGCGAGATAGGGGGG
CACGGGCGCGACCATCTGCGCTGCGGCGCCGGCGACTCAGCGCTGCCTCAGTCTGCGGT
GGGCAGCGGAGGAGTCGTGTCGTGCCTGAGAGCGCAGTCGAGAAGGTACCGGATCCGCC
ACCATGGTGAGCAAGGGCGAGGAGCTGTTCACCGGGGTGGTGCCCATCCTGGTCGAGCT
GGACGGCGACGTAAACGGCCACAAGTTCAGCGTGTCCGGCGAGGGCGAGGGCGATGCCA
CCTACGGCAAGCTGACCCTGAAGTTCATCTGCACCACCGGCAAGCTGCCCGTGCCCTGG
CCCACCCTCGTGACCACCCTGACCTACGGCGTGCAGTGCTTCAGCCGCTACCCCGACCA
CATGAAGCAGCACGACTTCTTCAAGTCCGCCATGCCCGAAGGCTACGTCCAGGAGCGCA
CCATCTTCTTCAAGGACGACGGCAACTACAAGACCCGCGCCGAGGTGAAGTTCGAGGGC
GACACCCTGGTGAACCGCATCGAGCTGAAGGGCATCGACTTCAAGGAGGACGGCAACAT
CCTGGGGCACAAGCTGGAGTACAACTACAACAGCCACAACGTCTATATCATGGCCGACA
AGCAGAAGAACGGCATCAAGGTGAACTTCAAGATCCGCCACAACATCGAGGACGGCAGC
GTGCAGCTCGCCGACCACTACCAGCAGAACACCCCCATCGGCGACGGCCCCGTGCTGCT
GCCCGACAACCACTACCTGAGCACCCAGTCCGCCCTGAGCAAAGACCCCAACGAGAAGC
GCGATCACATGGTCCTGCTGGAGTTCGTGACCGCCGCCGGGATCACTCTCGGCATGGAC
GAGCTGTACAAGTAGGAATTCGATATCAAGCTTATCGATAATCAACCTCTGGATTACAA
AATTTGTGAAAGATTGACTGGTATTCTTAACTATGTTGCTCCTTTTACGCTATGTGGAT
ACGCTGCTTTAATGCCTTTGTATCATGCTATTGCTTCCCGTATGGCTTTCATTTTCTCC
TCCTTGTATAAATCCTGGTTGCTGTCTCTTTATGAGGAGTTGTGGCCCGTTGTCAGGCA
ACGTGGCGTGGTGTGCACTGTGTTTGCTGACGCAACCCCCACTGGTTGGGGCATTGCCA
CCACCTGTCAGCTCCTTTCCGGGACTTTCGCTTTCCCCCTCCCTATTGCCACGGCGGAA
CTCATCGCCGCCTGCCTTGCCCGCTGCTGGACAGGGGCTCGGCTGTTGGGCACTGACAA
TTCCGTGGTGTTGTCGGGGAAATCATCGTCCTTTCCTTGGCTGCTCGCCTATGTTGCCA
CCTGGATTCTGCGCGGGACGTCCTTCTGCTACGTCCCTTCGGCCCTCAATCCAGCGGAC
CTTCCTTCCCGCGGCCTGCTGCCGGCTCTGCGGCCTCTTCCGCGTCTTCGCCTTCGCCC
TCAGACGAGTCGGATCTCCCTTTGGGCCGCCTCCCCGCATCGATACCGAGCGCTGCTCG
AGAGATCTACGGGTGGCATCCCTGTGACCCCTCCCCAGTGCCTCTCCTGGCCCTGGAAG
TTGCCACTCCAGTGCCCACCAGCCTTGTCCTAATAAAATTAAGTTGCATCATTTTGTCT
GACTAGGTGTCCTTCTATAATATTATGGGGTGGAGGGGGGTGGTATGGAGCAAGGGGCA
AGTTGGGAAGACAACCTGTAGGGCCTGCGGGGTCTATTGGGAACCAAGCTGGAGTGCAG
TGGCACAATCTTGGCTCACTGCAATCTCCGCCTCCTGGGTTCAAGCGATTCTCCTGCCT
CAGCCTCCCGAGTTGTTGGGATTCCAGGCATGCATGACCAGGCTCAGCTAATTTTTGTT
TTTTTGGTAGAGACGGGGTTTCACCATATTGGCCAGGCTGGTCTCCAACTCCTAATCTC
AGGTGATCTACCCACCTTGGCCTCCCAAATTGCTGGGATTACAGGCGTGAACCACTGCT
CCCTTCCCTGTCCTTCTGATTTTGTAGGTAACCACGTGCGGACCGAGCGGCCGCAGGAA
CCCCTAGTGATGGAGTTGGCCACTCCCTCTCTGCGCGCTCGCTCGCTCACTGAGGCCGG
GCGACCAAAGGTCGCCCGACGCCCGGGCTTTGCCCGGGCGGCCTCAGTGAGCGAGCGAG
CGCGCAGCTGCCTGCAGGGGCGCCTGATGCGGTATTTTCTCCTTACGCATCTGTGCGGT
ATTTCACACCGCATACGTCAAAGCAACCATAGTACGCGCCCTGTAGCGGCGCATTAAGC
GCGGCGGGTGTGGTGGTTACGCGCAGCGTGACCGCTACACTTGCCAGCGCCTTAGCGCC
CGCTCCTTTCGCTTTCTTCCCTTCCTTTCTCGCCACGTTCGCCGGCTTTCCCCGTCAAG
CTCTAAATCGGGGGCTCCCTTTAGGGTTCCGATTTAGTGCTTTACGGCACCTCGACCCC
AAAAAACTTGATTTGGGTGATGGTTCACGTAGTGGGCCATCGCCCTGATAGACGGTTTT
TCGCCCTTTGACGTTGGAGTCCACGTTCTTTAATAGTGGACTCTTGTTCCAAACTGGAA
CAACACTCAACTCTATCTCGGGCTATTCTTTTGATTTATAAGGGATTTTGCCGATTTCG
GTCTATTGGTTAAAAAATGAGCTGATTTAACAAAAATTTAACGCGAATTTTAACAAAAT
ATTAACGTTTACAATTTTATGGTGCACTCTCAGTACAATCTGCTCTGATGCCGCATAGT
TAAGCCAGCCCCGACACCCGCCAACACCCGCTGACGCGCCCTGACGGGCTTGTCTGCTC
CCGGCATCCGCTTACAGACAAGCTGTGACCGTCTCCGGGAGCTGCATGTGTCAGAGGTT
TTCACCGTCATCACCGAAACGCGCGAGACGAAAGGGCCTCGTGATACGCCTATTTTTAT
AGGTTAATGTCATGATAATAATGGTTTCTTAGACGTCAGGTGGCACTTTTCGGGGAAAT
GTGCGCGGAACCCCTATTTGTTTATTTTTCTAAATACATTCAAATATGTATCCGCTCAT
GAGACAATAACCCTGATAAATGCTTCAATAATATTGAAAAAGGAAGAGTATGAGTATTC
AACATTTCCGTGTCGCCCTTATTCCCTTTTTTGCGGCATTTTGCCTTCCTGTTTTTGCT
CACCCAGAAACGCTGGTGAAAGTAAAAGATGCTGAAGATCAGTTGGGTGCACGAGTGGG
TTACATCGAACTGGATCTCAACAGCGGTAAGATCCTTGAGAGTTTTCGCCCCGAAGAAC
GTTTTCCAATGATGAGCACTTTTAAAGTTCTGCTATGTGGCGCGGTATTATCCCGTATT
GACGCCGGGCAAGAGCAACTCGGTCGCCGCATACACTATTCTCAGAATGACTTGGTTGA
GTACTCACCAGTCACAGAAAAGCATCTTACGGATGGCATGACAGTAAGAGAATTATGCA
GTGCTGCCATAACCATGAGTGATAACACTGCGGCCAACTTACTTCTGACAACGATCGGA
GGACCGAAGGAGCTAACCGCTTTTTTGCACAACATGGGGGATCATGTAACTCGCCTTGA
TCGTTGGGAACCGGAGCTGAATGAAGCCATACCAAACGACGAGCGTGACACCACGATGC
CTGTAGCAATGGCAACAACGTTGCGCAAACTATTAACTGGCGAACTACTTACTCTAGCT
TCCCGGCAACAATTAATAGACTGGATGGAGGCGGATAAAGTTGCAGGACCACTTCTGCG
CTCGGCCCTTCCGGCTGGCTGGTTTATTGCTGATAAATCTGGAGCCGGTGAGCGTGGGT
CTCGCGGTATCATTGCAGCACTGGGGCCAGATGGTAAGCCCTCCCGTATCGTAGTTATC
TACACGACGGGGAGTCAGGCAACTATGGATGAACGAAATAGACAGATCGCTGAGATAGG
TGCCTCACTGATTAAGCATTGGTAACTGTCAGACCAAGTTTACTCATATATACTTTAGA
TTGATTTAAAACTTCATTTTTAATTTAAAAGGATCTAGGTGAAGATCCTTTTTGATAAT
CTCATGACCAAAATCCCTTAACGTGAGTTTTCGTTCCACTGAGCGTCAGACCCCGTAGA
AAAGATCAAAGGATCTTCTTGAGATCCTTTTTTTCTGCGCGTAATCTGCTGCTTGCAAA
CAAAAAAACCACCGCTACCAGCGGTGGTTTGTTTGCCGGATCAAGAGCTACCAACTCTT
TTTCCGAAGGTAACTGGCTTCAGCAGAGCGCAGATACCAAATACTGTTCTTCTAGTGTA
GCCGTAGTTAGGCCACCACTTCAAGAACTCTGTAGCACCGCCTACATACCTCGCTCTGC
TAATCCTGTTACCAGTGGCTGCTGCCAGTGGCGATAAGTCGTGTCTTACCGGGTTGGAC
TCAAGACGATAGTTACCGGATAAGGCGCAGCGGTCGGGCTGAACGGGGGGTTCGTGCAC
ACAGCCCAGCTTGGAGCGAACGACCTACACCGAACTGAGATACCTACAGCGTGAGCTAT
GAGAAAGCGCCACGCTTCCCGAAGGGAGAAAGGCGGACAGGTATCCGGTAAGCGGCAGG
GTCGGAACAGGAGAGCGCACGAGGGAGCTTCCAGGGGGAAACGCCTGGTATCTTTATAG
TCCTGTCGGGTTTCGCCACCTCTGACTTGAGCGTCGATTTTTGTGATGCTCGTCAGGGG
GGCGGAGCCTATGGAAAAACGCCAGCAACGCGGCCTTTTTACGGTTCCTGGCCTTTTGC
TGGCCTTTTGCTCA.

In some forms, the vector is a pUCmini-iCAP-PHP.eB scaffold vector that is used to package AAV PHP.eB. An exemplary scaffold vector is commercially available from Addgene, catalogue number 103005. Therefore, in some forms, the vector is a that is used to package AAV PHP.eB has a nucleic acid sequence of:

(SEQ ID NO: 8)
CCAATGATACGCGTCGGTGCGGGCCTCTTCGCTATTACGCCAGCTGGCGAAAGGGGGAT
GTGCTGCAAGGCGATTAAGTTGGGTAACGCCAGGGTTTTCCCAGTCACGACGTTGTAAA
ACGACGGCCAGTGAGCGCGCGTAATACGACTCACTATAGGGCGAATTGGGTACATCGAC
GGTATCGGGGGAGCTCGCAGGGTCTCCATTTTGAAGCGGGAGGTTTGAACGCGCAGCCG
CCATGCCGGGGTTTTACGAGATTGTGATTAAGGTCCCCAGCGACCTTGACGAGCATCTG
CCCGGCATTTCTGACAGCTTTGTGAACTGGGTGGCCGAGAAGGAATGGGAGTTGCCGCC
AGATTCTGACATGGATCTGAATCTGATTGAGCAGGCACCCCTGACCGTGGCCGAGAAGC
TGCAGCGCGACTTTCTGACGGAATGGCGCCGTGTGAGTAAGGCCCCGGAGGCTCTTTTC
TTTGTGCAATTTGAGAAGGGAGAGAGCTACTTCCACATGCACGTGCTCGTGGAAACCAC
CGGGGTGAAATCCATGGTTTTGGGACGTTTCCTGAGTCAGATTCGCGAAAAACTGATTC
AGAGAATTTACCGCGGGATCGAGCCGACTTTGCCAAACTGGTTCGCGGTCACAAAGACC
AGAAATGGCGCCGGAGGCGGGAACAAGGTGGTGGATGAGTGCTACATCCCCAATTACTT
GCTCCCCAAAACCCAGCCTGAGCTCCAGTGGGCGTGGACTAATATGGAACAGTATTTAA
GCGCCTGTTTGAATCTCACGGAGCGTAAACGGTTGGTGGCGCAGCATCTGACGCACGTG
TCGCAGACGCAGGAGCAGAACAAAGAGAATCAGAATCCCAATTCTGATGCGCCGGTGAT
CAGATCAAAAACTTCAGCCAGGTACATGGAGCTGGTCGGGTGGCTCGTGGACAAGGGGA
TTACCTCGGAGAAGCAGTGGATCCAGGAGGACCAGGCCTCATACATCTCCTTCAATGCG
GCCTCCAACTCGCGGTCCCAAATCAAGGCTGCCTTGGACAATGCGGGAAAGATTATGAG
CCTGACTAAAACCGCCCCCGACTACCTGGTGGGCCAGCAGCCCGTGGAGGACATTTCCA
GCAATCGGATTTATAAAATTTTGGAACTAAACGGGTACGATCCCCAATATGCGGCTTCC
GTCTTTCTGGGATGGGCCACGAAAAAGTTCGGCAAGAGGAACACCATCTGGCTGTTTGG
GCCTGCAACTACCGGGAAGACCAACATCGCGGAGGCCATAGCCCACACTGTGCCCTTCT
ACGGGTGCGTAAACTGGACCAATGAGAACTTTCCCTTCAACGACTGTGTGGACAAGATG
GTGATCTGGTGGGAGGAGGGGAAGATGACCGCCAAGGTCGTGGAGTCGGCCAAAGCCAT
TCTCGGAGGAAGCAAGGTGCGCGTGGACCAGAAATGCAAGTCCTCGGCCCAGATAGACC
CGACTCCCGTGATCGTCACCTCCAACACCAATATGTGCGCCGTGATTGACGGGAACTCA
ACGACCTTCGAACACCAGCAGCCGTTGCAAGACCGGATGTTCAAATTTGAACTCACCCG
CCGTCTGGATCATGACTTTGGGAAGGTCACCAAGCAGGAAGTCAAAGACTTTTTCCGGT
GGGCAAAGGATCACGTGGTTGAGGTGGAGCATGAATTCTACGTCAAAAAGGGTGGAGCC
AAGAAAAGACCCGCCCCCAGTGACGCAGATATAAGTGAGCCCAAACGGGTGCGCGAGTC
AGTTGCGCAGCCATCGACGTCAGACGCGGAAGCTTCGATCAACTACGCGGACAGGTACC
AAAACAAATGTTCTCGTCACGTGGGCATGAATCTGATGCTGTTTCCCTGCAGACAATGC
GAGAGACTGAATCAGAATTCAAATATCTGCTTCACTCACGGTGTCAAAGACTGTTTAGA
GTGCTTTCCCGTGTCAGAATCTCAACCCGTTTCTGTCGTCAAAAAGGCGTATCAGAAAC
TGTGCTACATTCATCACATCATGGGAAAGGTGCCAGACGCTTGCACTGCTTGCGACCTG
GTCAATGTGGACTTGGATGACTGTGTTTCTGAACAATAAATGACTTAAACCAGGTATGA
GTCGGCTGGATAAATCTAAAGTCATAAACGGCGCTCTGGAATTACTCAATGAAGTCGGT
ATCGAAGGCCTGACGACAAGGAAACTCGCTCAAAAGCTGGGAGTTGAGCAGCCTACCCT
GTACTGGCACGTGAAGAACAAGCGGGCCCTGCTCGATGCCCTGGCCATCGAGATGCTGG
ACAGGCATCATACCCACTTCTGCCCCCTGGAAGGCGAGTCATGGCAAGACTTTCTGCGG
AACAACGCCAAGTCATTCCGCTGTGCTCTCCTCTCACATCGCGACGGGGCTAAAGTGCA
TCTCGGCACCCGCCCAACAGAGAAACAGTACGAAACCCTGGAAAATCAGCTCGCGTTCC
TGTGTCAGCAAGGCTTCTCCCTGGAGAACGCACTGTACGCTCTGTCCGCCGTGGGCCAC
TTTACACTGGGCTGCGTATTGGAGGAACAGGAGCATCAAGTAGCAAAAGAGGAAAGAGA
GACACCTACCACCGATTCTATGCCCCCACTTCTGAGACAAGCAATTGAGCTGTTCGACC
GGCAGGGAGCCGAACCTGCCTTCCTTTTCGGCCTGGAACTAATCATATGTGGCCTGGAG
AAACAGCTAAAGTGCGAAAGCGGCGGGCCGGCCGACGCCCTTGACGATTTTGACTTAGA
CATGCTCCCAGCCGATGCCCTTGACGACTTTGACCTTGATATGCTGCCTGCTGACGCTC
TTGACGATTTTGACCTTGACATGCTCCCCGGGTAAATGCATGAATTCGATCTAGAGGGC
CCTATTCTATAGTGTCACCTAAATGCTAGAGCTCGCTGATCAGCCTCGACTGTGCCTTC
TAGTTGCCAGCCATCTGTTGTTTGCCCCTCCCCCGTGCCTTCCTTGACCCTGGAAGGTG
CCACTCCCACTGTCCTTTCCTAATAAAATGAGGAAATTGCATCGCATTGTCTGAGTAGG
TGTCATTCTATTCTGGGGGGTGGGGTGGGGCAGGACAGCAAGGGGGAGGATTGGGAAGA
CAATAGCAGGCATGCTGGGGATGCGGTGGGCTCTATGGCTTCTGAGGCGGAAAGAACCA
GCTGGGGCTCGAATCAAGCTATCAAGTGCCACCTGACGTCTCCCTATCAGTGATAGAGA
AGTCGACACGTCTCGAGCTCCCTATCAGTGATAGAGAAGGTACGTCTAGAACGTCTCCC
TATCAGTGATAGAGAAGTCGACACGTCTCGAGCTCCCTATCAGTGATAGAGAAGGTACG
TCTAGAACGTCTCCCTATCAGTGATAGAGAAGTCGACACGTCTCGAGCTCCCTATCAGT
GATAGAGAAGGTACGTCTAGAACGTCTCCCTATCAGTGATAGAGAAGTCGACACGTCTC
GAGCTCCCTATCAGTGATAGAGAAGGTACCCCCTATATAAGCAGAGAGATCTGTTCAAA
TTTGAACTGACTAAGCGGCTCCCGCCAGATTTTGGCAAGATTACTAAGCAGGAAGTCAA
GGACTTTTTTGCTTGGGCAAAGGTCAATCAGGTGCCGGTGACTCACGAGTTTAAAGTTC
CCAGGGAATTGGCGGGAACTAAAGGGGCGGAGAAATCTCTAAAACGCCCACTGGGTGAC
GTCACCAATACTAGCTATAAAAGTCTGGAGAAGCGGGCCAGGCTCTCATTTGTTCCCGA
GACGCCTCGCAGTTCAGACGTGACTGTTGATCCCGCTCCTCTGCGACCGCTAGCTTCGA
TCAACTACGCAGACAGGTACCAAAACAAGTGTTCTCGTCACGTGGGCATTAATCTGATT
CTGTTTCCCTGCAGACAATGCGAGAGAATGAATCAGAACTCAAATATCTGCTTCACTCA
CGGACAGAAAGACTGTTTAGAGTGCTTTCCCGTGTCAGAATCTCAACCCGTTTCTGTCG
TCAAAAAGGCGTATCAGAAACTGTGCTACATTCATCATATCATGGGAAAGGTGCCAGAC
GCTTGCACTGCCTGCGATCTGGTCAATGTGGATTTGGATGACTGCATCTTTGAACAATA
AATGACTTAAGCCAGGTATGGCTGCCGATGGTTATCTTCCAGATTGGCTCGAGGACAAC
CTTAGTGAAGGAATTCGCGAGTGGTGGGCTTTGAAACCTGGAGCCCCTCAACCCAAGGC
AAATCAACAACATCAAGACAACGCTAGAGGTCTTGTGCTTCCGGGTTACAAATACCTTG
GACCCGGCAACGGACTCGACAAGGGGGAGCCGGTCAACGCAGCAGACGCGGCGGCCCTC
GAGCACGACAAAGCCTACGACCAGCAGCTCAAGGCCGGAGACAACCCGTACCTCAAGTA
CAACCACGCCGACGCCGAGTTCCAGGAGCGGCTCAAAGAAGATACGTCTTTTGGGGGCA
ACCTCGGGCGAGCAGTCTTCCAGGCCAAAAAGAGGCTTCTTGAACCTCTTGGTCTGGTT
GAGGAAGCGGCTAAGACGGCTCCTGGAAAGAAGAGGCCTGTAGAGCAGTCTCCTCAGGA
ACCGGACTCCTCCGCGGGTATTGGCAAATCGGGTGCACAGCCCGCTAAAAAGAGACTCA
ATTTCGGTCAGACTGGCGACACAGAGTCAGTCCCAGACCCTCAACCAATCGGAGAACCT
CCCGCAGCCCCCTCAGGTGTGGGATCTCTTACAATGGCTTCAGGTGGTGGCGCACCAGT
GGCAGACAATAACGAAGGTGCCGATGGAGTGGGTAGTTCCTCGGGAAATTGGCATTGCG
ATTCCCAATGGCTGGGGGACAGAGTCATCACCACCAGCACCCGAACCTGGGCCCTGCCC
ACCTACAACAATCACCTCTACAAGCAAATCTCCAACAGCACATCTGGAGGATCTTCAAA
TGACAACGCCTACTTCGGCTACAGCACCCCCTGGGGGTATTTTGACTTCAACAGATTCC
ACTGCCACTTCTCACCACGTGACTGGCAGCGACTCATCAACAACAACTGGGGATTCCGG
CCTAAGCGACTCAACTTCAAGCTCTTTAACATTCAGGTCAAAGAGGTTACGGACAACAA
TGGAGTCAAGACCATCGCCAATAACCTTACCAGCACGGTCCAGGTCTTCACGGACTCAG
ACTATCAGCTCCCGTACGTGCTCGGGTCGGCTCACGAGGGCTGCCTCCCGCCGTTCCCA
GCGGACGTTTTCATGATTCCTCAGTACGGGTATCTGACGCTTAATGATGGAAGCCAGGC
CGTGGGTCGTTCGTCCTTTTACTGCCTGGAATATTTCCCGTCGCAAATGCTAAGAACGG
GTAACAACTTCCAGTTCAGCTACGAGTTTGAGAACGTACCTTTCCATAGCAGCTACGCT
CACAGCCAAAGCCTGGACCGACTAATGAATCCACTCATCGACCAATACTTGTACTATCT
CTCTAGAACTATTAACGGTTCTGGACAGAATCAACAAACGCTAAAATTCAGTGTGGCCG
GACCCAGCAACATGGCTGTCCAGGGAAGAAACTACATACCTGGACCCAGCTACCGACAA
CAACGTGTCTCAACCACTGTGACTCAAAACAACAACAGCGAATTTGCTTGGCCTGGAGC
TTCTTCTTGGGCTCTCAATGGACGTAATAGCTTGATGAATCCTGGACCTGCTATGGCCT
CTCACAAAGAAGGAGAGGACCGTTTCTTTCCTTTGTCTGGATCTTTAATTTTTGGCAAA
CAAGGTACTGGCAGAGACAACGTGGATGCGGACAAAGTCATGATAACCAACGAAGAAGA
AATTAAAACTACTAACCCGGTAGCAACGGAGTCCTATGGACAAGTGGCCACAAACCACC
AGAGTGATGGGACTTTGGCGGTGCCTTTTAAGGCACAGGCGCAGACCGGTTGGGTTCAA
AACCAAGGAATACTTCCGGGTATGGTTTGGCAGGACAGAGATGTGTACCTGCAAGGACC
CATTTGGGCCAAAATTCCTCACACGGACGGCAACTTTCACCCTTCTCCGCTGATGGGAG
GGTTTGGAATGAAGCACCCGCCTCCTCAGATCCTCATCAAAAACACACCTGTACCTGCG
GATCCTCCAACGGCCTTCAACAAGGACAAGCTGAACTCTTTCATCACCCAGTATTCTAC
TGGTCAAGTCAGCGTGGAGATCGAGTGGGAGCTGCAGAAGGAAAACAGCAAGCGCTGGA
ACCCGGAGATCCAGTACACTTCCAACTATTACAAGTCTAATAATGTTGAATTTGCTGTT
AATACTGAAGGTGTATATAGTGAACCCCGCCCCATTGGCACCAGATACCTGACTCGTAA
TCTGTAAGTCGACTTGCTTGTTAATCAATAAACCGTTTAATTCGTTTCAGTTGAACTTT
GGTCTCTGCGAAGGGCAATTCGTTTAAACCTGCAGGACTAGAGGTCCTGTATTAGAGGT
CACGTGAGTGTTTTGCGACATTTTGCGACACCATGTGGTCACGCTGGGTATTTAAGCCC
GAGTGAGCACGCAGGGTCTCCATTTTGAAGCGGGAGGTTTGAACGCGCAGCCGCCAAGC
CGAATTCTGCAGATATCACATGTCCTAGGAACTATCGATCCATCACACTGGCGGCCGCT
CGACTAGAGCGGCCGCCACCGCGGTGGAGCTCCAGCTTTTGCGGACCGAATCGGAAAGA
ACATGTGAGCAAAAGGCCAGCAAAAGGCCAGGAACCGTAAAAAGGCCGCGTTGCTGGCG
TTTTTCCATAGGCTCCGCCCCCCTGACGAGCATCACAAAAATCGACGCTCAAGTCAGAG
GTGGCGAAACCCGACAGGACTATAAAGATACCAGGCGTTTCCCCCTGGAAGCTCCCTCG
TGCGCTCTCCTGTTCCGACCCTGCCGCTTACCGGATACCTGTCCGCCTTTCTCCCTTCG
GGAAGCGTGGCGCTTTCTCATAGCTCACGCTGTAGGTATCTCAGTTCGGTGTAGGTCGT
TCGCTCCAAGCTGGGCTGTGTGCACGAACCCCCCGTTCAGCCCGACCGCTGCGCCTTAT
CCGGTAACTATCGTCTTGAGTCCAACCCGGTAAGACACGACTTATCGCCACTGGCAGCA
GCCACTGGTAACAGGATTAGCAGAGCGAGGTATGTAGGCGGTGCTACAGAGTTCTTGAA
GTGGTGGCCTAACTACGGCTACACTAGAAGAACAGTATTTGGTATCTGCGCTCTGCTGA
AGCCAGTTACCTTCGGAAAAAGAGTTGGTAGCTCTTGATCCGGCAAACAAACCACCGCT
GGTAGCGGTGGTTTTTTTGTTTGCAAGCAGCAGATTACGCGCAGAAAAAAAGGATCTCA
AGAAGATCCTTTGATCTTTTCTACGGGGTCTGACGCTCAGTGGAACGAAAACTCACGTT
AAGGGATTTTGGTCATGAGATTATCAAAAAGGATCTTCACCTAGATCCTTTTAAATTAA
AAATGAAGTTTTAAATCAATCTAAAGTATATATGAGTAAACTTGGTCTGACAGTTACCA
ATGCTTAATCAGTGAGGCACCTATCTCAGCGATCTGTCTATTTCGTTCATCCATAGTTG
CCTGACTCCCCGTCGTGTAGATAACTACGATACGGGAGGGCTTACCATCTGGCCCCAGT
GCTGCAATGATACCGCGAGACCCACGCTCACCGGCTCCAGATTTATCAGCAATAAACCA
GCCAGCCGGAAGGGCCGAGCGCAGAAGTGGTCCTGCAACTTTATCCGCCTCCATCCAGT
CTATTAATTGTTGCCGGGAAGCTAGAGTAAGTAGTTCGCCAGTTAATAGTTTGCGCAAC
GTTGTTGCCATTGCTACAGGCATCGTGGTGTCACGCTCGTCGTTTGGTATGGCTTCATT
CAGCTCCGGTTCCCAACGATCAAGGCGAGTTACATGATCCCCCATGTTGTGCAAAAAAG
CGGTTAGCTCCTTCGGTCCTCCGATCGTTGTCAGAAGTAAGTTGGCCGCAGTGTTATCA
CTCATGGTTATGGCAGCACTGCATAATTCTCTTACTGTCATGCCATCCGTAAGATGCTT
TTCTGTGACTGGTGAGTACTCAACCAAGTCATTCTGAGAATAGTGTATGCGGCGACCGA
GTTGCTCTTGCCCGGCGTCAATACGGGATAATACCGCGCCACATAGCAGAACTTTAAAA
GTGCTCATCATTGGAAAACGTTCTTCGGGGCGAAAACTCTCAAGGATCTTACCGCTGTT
GAGATCCAGTTCGATGTAACCCACTCGTGCACCCAACTGATCTTCAGCATCTTTTACTT
TCACCAGCGTTTCTGGGTGAGCAAAAACAGGAAGGCAAAATGCCGCAAAAAAGGGAATA
AGGGCGACACGGAAATGTTGAATACTCATACTCTTCCTTTTTCAATATTATTGAAGCAT
TTATCAGGGTTATTGTCTCATGAGCGGATACATATTTGAATGTATTTAGAAAAATAAAC
AAATAGGGGTTCCGCGCACATTTCCCCGAAAAGTGCCACCTGACGTC.

### 2. Other Viral Vectors

In some forms, the DLC1 gene is packaged for delivery to cells within or otherwise associated with a non-AAV viral vector. Suitable viral vectors include, without limitation, vectors derived from bacteriophages, baculoviruses, retroviruses (such as lentiviruses), adenoviruses, poxviruses, and Epstein-Barr viruses. In some forms, the viral vector is derived from a DNA virus (e.g., dsDNA or ssDNA virus) or an RNA virus (e.g., an ssRNA virus). Numerous vectors and expression systems are commercially available from commercial vendors including Addgene, Novagen (Madison, WI), Clontech (Palo Alto, CA), Stratagene (La Jolla, CA), and Invitrogen/Life Technologies (Carlsbad, CA).

In some forms, the vector is a viral vector such as a vesicular stomatitis (VSV) vector, a Bocavirus vector, such as a human bocavirus 1 (HBoV1) vector, a Herpes simplex virus (HSV) vector, or an adenovirus vector (AdV).

In some forms, the viral vector is a Herpes simplex virus (HSV) vector. Herpes simplex viruses (HSV) are large, enveloped dsDNA viruses characteristic of their lytic and latent nature of infection, which result in life-long latent infection of neurons and allows for long-term transgene expression. Deletion of HSV genes has generated expression vectors with low toxicity and an excellent packaging capacity of >30 kb foreign DNA. In some forms, the viral vector is a Vesicular stomatitis virus (VSV) vector. Vesicular stomatitis virus is a non-segmented, negative-stranded RNA virus that belongs to the family Rhabdoviridae, genus Vesiculovirus. VSV infects a broad range of animals, including cattle, horses, and swine. The genome of the virus codes for five major proteins, glycoprotein (G), matrix protein (M), nucleoprotein (N), large protein (L), and phosphoprotein (P). The G protein mediates both viral binding and host cell fusion with the endosomal membrane following endocytosis. The L and P proteins are subunits of the viral RNA-dependent RNA polymerase. The simple structure and rapid high-titer growth of VSV in mammalian and many other cells has made recombinant VSV a useful tool in the fields of cellular and molecular biology and virology.

In some forms, the viral vector is a human Bocavirus vector (HBoV). Exemplary human bocavirus vectors include human bocaviruses 1-4 (HBoV1-4), As well as Gorilla BoV.

In other forms, the viral vector is an adenovirus vector that is not a typical AAV. In some forms, the vector is a chimeric vector, such as a vector that is based on a chimeric virus formed from a combination of one or more components from two or more different viral vectors. An exemplary chimeric viral vector is a chimeric bocavirus/adeno-associated virus vector. Therefore, in some forms, the vector is a chimeric HBoV1/AAV2 vector (e.g., rAAV2/HBoV1 chimeras).

### C. Promoters

In some forms, the transgene (e.g., the DLC1 transgene) is operably linked to a promoter. Useful promoters are strong promoters that drive expression of the associated proteins in the brain and CNS. A preferred promoter is the human synapsin 1 gene promoter (SYN promoter) (Kugler, et al., Gene Ther 10, 337-347 (2003)). SYN is a general neuronal promoter and it has previously been shown that the rAAV2/1-SYN system can be used to achieve expression in both excitatory and inhibitory neurons in neocortex (Nathanson et al., 2009). In cerebellar cortex, however, we find that SYN is highly heterogeneous with respect to cell type.

Other exemplary promoters include, but are not limited to, the cytomegalovirus (CMV) immediate early promoter, the RSV LTR, the MoMLV LTR, the phosphoglycerate kinase-I (PGK) promoter, a simian virus 40 (SV40) promoter and a CK6 promoter, a transthyretin promoter (TTR), a TK promoter, a tetracycline responsive promoter (TRE), an HBV promoter, an hAAT promoter, a LSP promoter, chimeric liver-specific promoters (LSPs), the E2F promoter, the telomerase (hTERT) promoter; the cytomegalovirus enhancer/chicken beta-actin/Rabbit ˜-globin promoter (CAG promoter; Niwa et al., Gene, 1991, 108(2):193-9) and the elongation factor I-alpha promoter (EFI-alpha) promoter (Guo et al., Gene Ther., 1996, 3(9): 802-10). In some forms, the promoter includes a human β-glucuronidase promoter or a cytomegalovirus enhancer linked to a chicken β-actin promoter. The promoter can be a constitutive, inducible or repressible promoter. In some forms, the transgene encodes any DLC1 and optionally also a SMN protein described herein (e.g., primate DLC1 and SMN) and is operable linked to a promoter. In some forms, the trans gene encodes a human DLC1 protein and is operable linked to a promoter. In some forms, the transgene encodes a human DLC1 protein including the amino acid sequence of SEQ ID NO:3 and is operable linked to a promoter. In some forms, the transgene includes a nucleic acid of SEQ ID NO: 1 and is operable linked to a promoter.

In some forms, the hSYN promoter has a nucleic acid sequence of:

(SEQ ID NO: 9)
AGTGCAAGTGGGTTTTAGGACCAGGATGAGGCGGGGTGGGGGTGCCTACCTGACGACCG
ACCCCGACCCACTGGACAAGCACCCAACCCCCATTCCCCAAATTGCGCATCCCCTATCA
GAGAGGGGGAGGGGAAACAGGATGCGGCGAGGCGCGTGCGCACTGCCAGCTTCAGCACC
GCGGACAGTGCCTTCGCCCCCGCCTGGCGGCGCGCGCCACCGCCGCCTCAGCACTGAAG
GCGCGCTGACGTCACTCGCCGGTCCCCCGCAAACTCCCCTTCCCGGCCACCTTGGTCGC
GTCCGCGCCGCCGCCGGCCCAGCCGGACCGCACCACGCGAGGCGCGAGATAGGGGGGCA
CGGGCGCGACCATCTGCGCTGCGGCGCCGGCGACTCAGCGCTGCCTCAGTCTGCGGTGG
GCAGCGGAGGAGTCGTGTCGTGCCTGAGAGCGCAGAGTGCAAGTGGGTTTTAGGACCAG
GATGAGGCGGGGTGGGGGTGCCTACCTGACGACCGACCCCGACCCACTGGACAAGCACC
CAACCCCCATTCCCCAAATTGCGCATCCCCTATCAGAGAGGGGGAGGGGAAACAGGATG
CGGCGAGGCGCGTGCGCACTGCCAGCTTCAGCACCGCGGACAGTGCCTTCGCCCCCGCC
TGGCGGCGCGCGCCACCGCCGCCTCAGCACTGAAGGCGCGCTGACGTCACTCGCCGGTC
CCCCGCAAACTCCCCTTCCCGGCCACCTTGGTCGCGTCCGCGCCGCCGCCGGCCCAGCC
GGACCGCACCACGCGAGGCGCGAGATAGGGGGGCACGGGCGCGACCATCTGCGCTGCGG
CGCCGGCGACTCAGCGCTGCCTCAGTCTGCGGTGGGCAGCGGAGGAGTCGTGTCGTGCC
TGAGAGCGCAG.

### D. Additional Agents

In some forms, the compositions include one or more additional molecules that is an active agent. Exemplary active agents include therapeutic, diagnostic, nutraceutical and prophylactic agents. In some forms, the compositions include one or more additional therapeutic agents that are active in the treatment and/or diagnosis of Spinal muscular atrophy (SMA).

Exemplary additional therapeutic agents include FDA approved medications to treat SMA. Exemplary medications include nusinersen and onasemnogene abeparvovec-xioi.

Nusinersen is FDA-approved for both pediatric and adult patients with genetically confirmed SMA. This medication is given as an injection into the spinal canal for treatment of different types of SMAs, including types 3 and 4. Therefore, in some forms, the compositions include Nusinersen.

Onasemnogene abeparvovec-xioi (ZOLGENSMA™) is a gene therapy medication for treating SMA, and is a one-time intravenous infusion for pediatric patients. Therefore, in some forms, the compositions include onasemnogene abeparvovec-xioi.

### E. Pharmaceutical Excipients

In some forms, compositions including recombinant AAV encapsidating an expression vector encoding a DLC1-i1 gene or a DLC1-i1 protein (rAAV-DLC1-i1) include one or more other pharmaceutically acceptable carriers, including any suitable diluent or excipient.

For intradermal injection, or injection at the site of affliction, the compositions including rAAV-DLC1-i1 are typically formulated in the form of a parenterally acceptable aqueous solution which is pyrogen-free and has suitable pH, isotonicity, and stability. Those of relevant skill in the art are well able to prepare suitable solutions using, for example, isotonic vehicles such as Sodium Chloride Injection, Ringer's Injection, or Lactated Ringer's Injection. Preservatives, stabilizers, buffers, antioxidants, and/or other additives can be included, as required. rAAV-DLC1-i1 is formulated in sodium chloride solution and will be injected through a vein.

Administration is preferably in a “therapeutically effective amount”, this being sufficient to show benefit to the individual. The actual amount administered, and rate and time-course of administration, will depend on the nature and severity of disease being treated. Prescription of treatment, e.g., decisions on dosage etc., is within the responsibility of general practitioners and other medical doctors, and typically takes account of the disorder to be treated, the condition of the individual patient, the site of delivery, the method of administration and other factors known to practitioners.

Preferably, the pharmaceutically acceptable carrier does not itself induce a physiological response, e.g., an immune response, nor result in any adverse or undesired side effects and/or does not result in undue toxicity. Pharmaceutically acceptable carriers include, but are not limited to, saline, buffered saline, dextrose, water, glycerol, sterile isotonic aqueous buffer, and combinations thereof. Additional examples of pharmaceutically acceptable carriers, diluents, and excipients are provided in Remington's Pharmaceutical Sciences (Mack Pub. Co., N.J., current edition).

### F. Exemplary Compositions Including DLC1-i1

In some forms, the compositions include a recombinant AAV particle including a nucleic acid sequence encoding a DLC1-i1 gene (rAAV-DLC1-i1), optionally further including a nucleic acid sequence encoding a SMN gene (e.g., human SMN protein) (rAAV-DLC1-i1/SMN1). Typically the gene(s) are flanked by one or two ITRs. Typically, the nucleic acid is encapsidated in the AAV particle. The AAV particle also includes capsid proteins. In some forms, the nucleic acid includes the protein coding sequence(s) of interest (e.g., a transgene encoding an DLC1-i1 protein and optionally also a transgene encoding an SMN protein) operatively linked components in the direction of transcription, control sequences including transcription initiation and termination sequences, thereby forming an expression cassette. An exemplary expression cassette is flanked on the 5′ and 3′ end by at least one functional AAV ITR sequences. The term “functional AAV ITR sequences” infer that the ITR sequences function as intended for the rescue, replication and packaging of the AAV virion.

In some forms, the expression of the encapsidated genes encoding the DLC1-i1 and optionally the SMN1 transgenes is under the control of the human synapsin 1 gene promoter (SYN promoter) (rAAV-SYN-DLC1-i1/SMN1).

Generally, the recombinant vectors include at least all of the sequences of AAV essential for encapsidation and the physical structures for infection by the AAV. AAV ITRs for use in the vectors of the invention need not have a wild-type nucleotide sequence (e.g., as described in Katin, Hum. Gene Ther., 1994, 5:793-801), and may be altered by the insertion, deletion or substitution of nucleotides or the AAV ITRs may be derived from any of several AAV serotypes. More than 40 serotypes of AAV are currently known, and new serotypes and variants of existing serotypes continue to be identified (Gao et al., PNAS, 2002, 99(18): 11854-6; Gao et al., PNAS, 2003, 100(10): 6081-6; and Bossis et al., J. Viral., 2003, 77(12):6799-810).

In some forms, the AAV vector is a vector derived from an AAV serotype, including without limitation, AAV1, AAV2, AAV3, AAV4, AAV5, AA6, AAV7, AAV8, AAV9, AAVrh.8, AAVrh. 10, AAV11, or AAV12. In some forms, the nucleic acid in the AAV includes an ITR of AAV1, AAV2, AAV3, AAV4, AAV5, AAV6, AAV7, AAV8, AAV9, AAVrh.8, AAVrh.10, AAV11, or AAV12. In some forms, the nucleic acid in the AAV further encodes any one or more DLC1-i1 protein, and optionally a SMN protein as described herein. For example, the nucleic acid in the AAV can include at least one ITR of any AAV serotype contemplated herein and can further include a DLC1-i1 gene having the nucleic acid sequence of SEQ ID NO: 1, or a protein including the amino acid of SEQ ID NO:2, and optionally an SMN1 gene having the nucleic acid sequence of SEQ ID NO:3, or a protein including the amino acid of SEQ ID NO:4.

In some forms, the composition is formulated for administration to a subject in vivo. For example, in some forms, the composition is formulated for intracerebroventricular (ICV) injection. In some forms, the composition includes rAAV-SYN-DLC1-i1/SMN1. In other forms, the composition includes rAAV-SYN-DLC1-i1 and rAAV-SYN-SMN1 as an admixture within the same formulation. The amounts of rAAV-SYN-DLC1-i1 and rAAV-SYN-SMN1 within the composition can be the same or different. For example, in some forms, the molar ratio or mass ratio of rAAV-SYN-DLC1-i1 to rAAV-SYN-SMN1 within the formulation is 1:1, 1:2, 1:3, 1:4, 1:5, 1:6, 1:7, 1:8, 1:9 or 1:10, or 10:1, 9:1, 8:1, 7:1, 6:1, 5:1, 4:1, 3:1, 2:1 or 100:1, or more than 100:1.

### 1. Exemplary DLC1-i1/AAV/SYN and SMN1/AAV/SYN Vectors

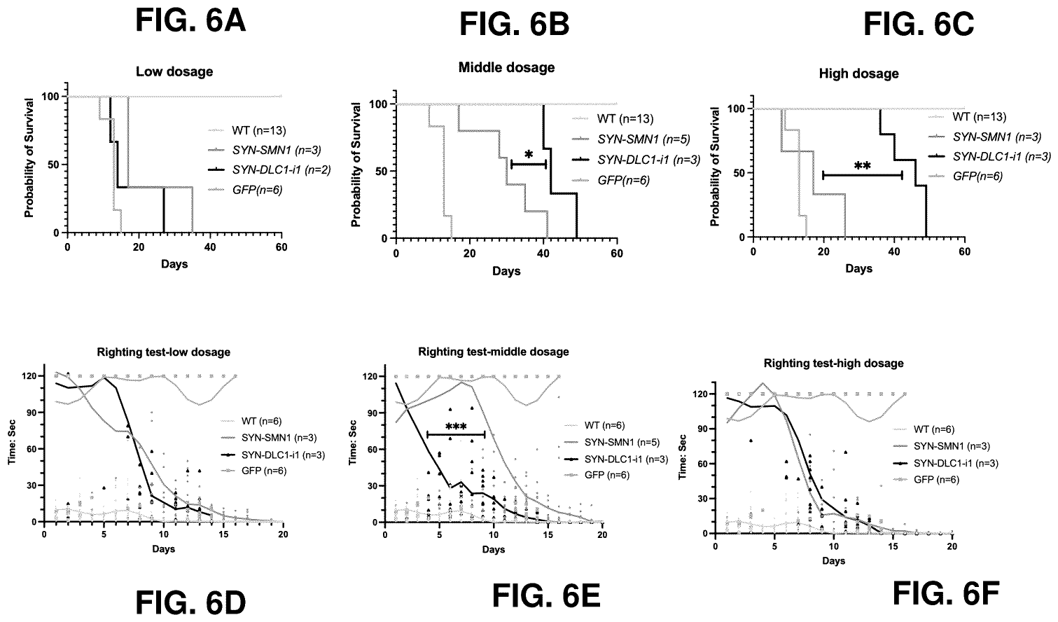

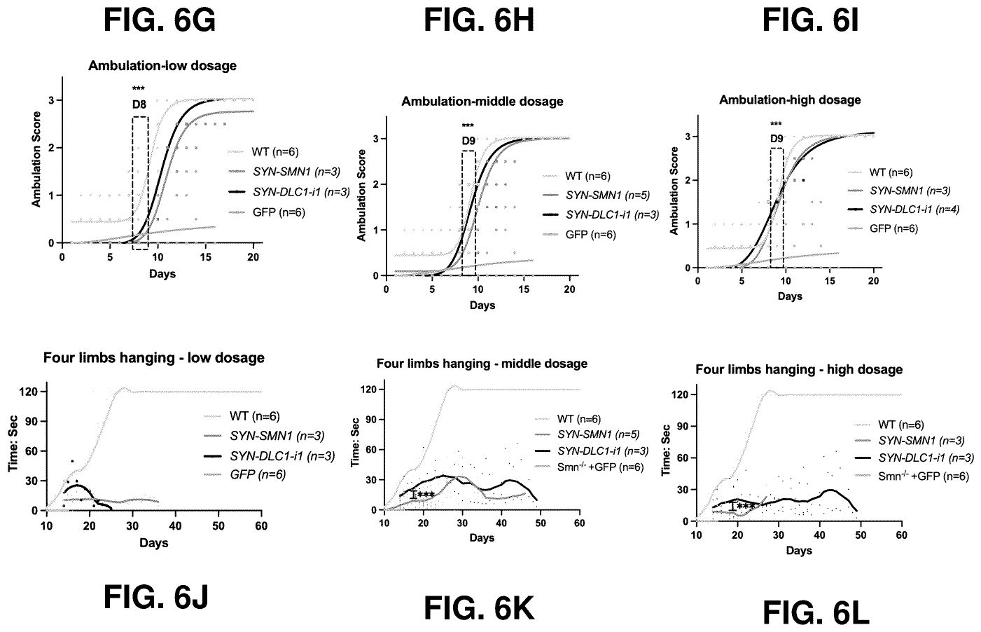

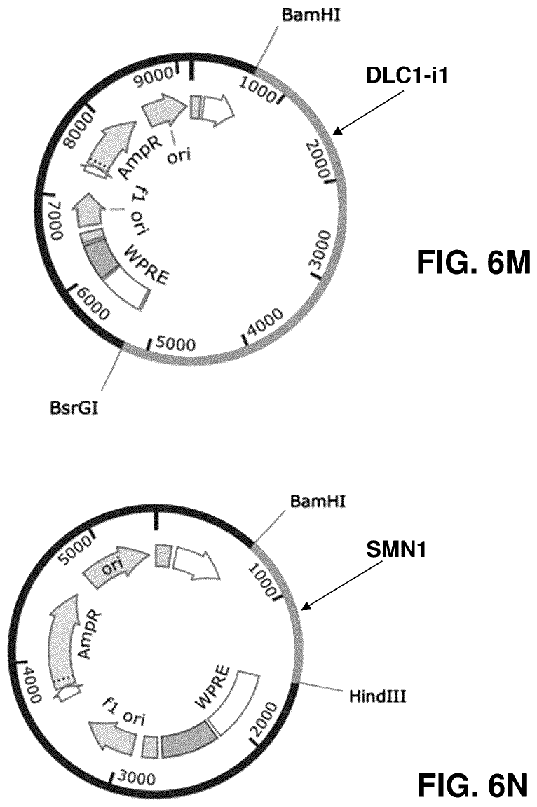

In some forms, the DLC1-i1 gene is contained within a AAV vector configured for expression of the DLC1-i1 protein in a target cell under the control of the SYN promoter. An exemplary vector is a hSyn-DLC1-i1 AAV plasmid. In some forms, the hSyn-DLC1-i1 AAV plasmid is constructed by pAAV-hSyn-EGFP and cDNA of DLC1-i1. For example, in some forms, the plasmid is hSYN-DLC1-i1 AAV having 9137 base pairs (bp), as depicted in FIG. 6M. In some forms, the hSyn-DLC1-i1 AAV includes the nucleic acid sequence:

CATGTCCTGCAGGCAGCTGCGCGCTCGCTCGCTCACTGAGGCCGCCCGGGCGTCGGGCG
ACCTTTGGTCGCCCGGCCTCAGTGAGCGAGCGAGCGCGCAGAGAGGGAGTGGCCAACTC
CATCACTAGGGGTTCCTGCGGCCGCACGCGTGTGTCTAGACTGCAGAGGGCCCTGCGTA
TGAGTGCAAGTGGGTTTTAGGACCAGGATGAGGCGGGGTGGGGGTGCCTACCTGACGAC
CGACCCCGACCCACTGGACAAGCACCCAACCCCCATTCCCCAAATTGCGCATCCCCTAT
CAGAGAGGGGGAGGGGAAACAGGATGCGGCGAGGCGCGTGCGCACTGCCAGCTTCAGCA
CCGCGGACAGTGCCTTCGCCCCCGCCTGGCGGCGCGCGCCACCGCCGCCTCAGCACTGA
AGGCGCGCTGACGTCACTCGCCGGTCCCCCGCAAACTCCCCTTCCCGGCCACCTTGGTC
GCGTCCGCGCCGCCGCCGGCCCAGCCGGACCGCACCACGCGAGGCGCGAGATAGGGGGG
CACGGGCGCGACCATCTGCGCTGCGGCGCCGGCGACTCAGCGCTGCCTCAGTCTGCGGT
GGGCAGCGGAGGAGTCGTGTCGTGCCTGAGAGCGCAGTCGAGAAGGTACCGGATCCATG
TCTGTAGCTATCAGAAAGAGAAGCTGGGAAGAACATGTGACCCACTGGATGGGACAGCC
TTTTAATTCTGATGATCGTAACACAGCATGTCATCATGGACTAGTAGCTGACAGCTTGC
AGGCAAGTATGGAAAAAGATGCAACTCTAAATGTGGACCGCAAAGAGAAGTGTGTTTCA
CTACCTGACTGCTGTCATGGATCAGAGCTGAGAGATTTTCCTGGGAGGCCAATGGGTCA
TCTTTCAAAGGATGTGGACGAAAATGACAGCCATGAAGGTGAAGATCAGTTTCTTTCTC
TGGAAGCCAGCACAGAAACACTAGTGCATGTTTCTGATGAGGATAACAATGCTGATTTA
TGCCTTACAGATGATAAACAGGTTTTAAATACCCAAGGGCAGAAAACATCAGGCCAACA
TATGATCCAAGGAGCAGGCTCCTTAGAAAAGGCACTGCCCATCATACAAAGTAACCAAG
TTTCTTCTAACTCCTGGGGAATAGCTGGTGAAACTGAATTAGCACTGGTAAAAGAAAGT
GGGGAGAGAAAAGTTACTGACTCTATAAGTAAAAGCCTGGAGCTTTGCAATGAAATAAG
CTTAAGTGAAATAAAAGATGCACCCAAAGTAAATGCAGTGGATACTTTGAACGTGAAAG
ATATTGCACCTGAGAAACAATTGCTTAACTCTGCTGTAATTGCTCAGCAACGAAGGAAA
CCTGACCCCCCTAAAGATGAAAATGAAAGAAGCACCTGCAATGTAGTACAAAATGAGTT
CTTGGATACTCCTTGCACAAACAGAGGACTGCCATTATTAAAAACAGATTTTGGAAGCT
GCCTTCTGCAGCCTCCTTCCTGCCCCAATGGAATGTCAGCTGAAAATGGCCTGGAGAAG
AGTGGTTTTTCACAACATCAAAACAAAAGTCCACCAAAGGTCAAGGCAGAAGATGGCAT
GCAGTGTTTACAATTAAAGGAGACCCTGGCCACCCAGGAACCCACAGATAACCAAGTCA
GACTTCGTAAGAGAAAGGAAATAAGAGAAGATCGAGATAGGGCGCGGCTGGACTCCATG
GTGCTGCTGATTATGAAACTGGACCAGCTTGATCAGGACATAGAAAATGCCCTCAGCAC
CAGCTCCTCTCCATCAGGCACACCAACAAACCTGCGGCGGCACGTTCCTGATCTGGAAT
CAGGATCTGAAAGTGGAGCAGATACCATTTCAGTAAATCAGACACGAGTAAATTTGTCT
TCTGACACTGAGTCCACGGACCTCCCATCTTCCACTCCAGTAGCCAATTCTGGAACCAA
ACCCAAGACTACGGCTATTCAAGGTATTTCAGAGAAGGAAAAGGCTGAAATTGAAGCCA
AGGAAGCTTGTGATTGGCTACGGGCAACTGGTTTCCCCCAGTATGCACAGCTTTATGAA
GATTTCCTGTTCCCCATCGATATTTCCTTGGTCAAGAGAGAGCATGATTTTTTGGACAG
AGATGCCATTGAGGCTCTATGCAGGCGTCTAAATACTTTAAACAAATGTGCGGTGATGA
AGCTAGAAATTAGTCCTCATCGGAAACGAAGTGACGATTCAGACGAGGATGAGCCTTGT
GCCATCAGTGGCAAATGGACTTTCCAAAGGGACAGCAAGAGGTGGTCCCGGCTTGAAGA
GTTTGATGTCTTTTCTCCAAAACAAGACCTGGTCCCTGGGTCCCCAGACGACTCCCACC
CGAAGGACGGCCCCAGCCCCGGAGGCACGCTGATGGACCTCAGCGAGCGCCAGGAGGTG
TCTTCCGTCCGCAGCCTCAGCAGCACTGGCAGCCTCCCCAGCCACGCGCCCCCCAGCGA
GGATGCTGCCACCCCCCGGACTAACTCCGTCATCAGCGTTTGCTCCTCCAGCAACTTGG
CAGGCAATGACGACTCTTTCGGCAGCCTGCCCTCTCCCAAGGAACTGTCCAGCTTCAGC
TTCAGCATGAAAGGCCACGAAAAAACTGCCAAGTCCAAGACGCGCAGTCTGCTGAAACG
GATGGAGAGCCTGAAGCTCAAGAGCTCCCATCACAGCAAGCACAAAGCGCCCTCAAAGC
TGGGGTTGATCATCAGCGGGCCCATCTTGCAAGAGGGGATGGATGAGGAGAAGCTGAAG
CAGCTCAACTGCGTGGAGATCTCCGCCCTCAATGGCAACCGCATCAACGTCCCCATGGT
ACGAAAGAGGAGCGTTTCCAACTCCACGCAGACCAGCAGCAGCAGCAGCCAGTCGGAGA
CCAGCAGCGCGGTCAGCACGCCCAGCCCTGTTACGAGGACCCGGAGCCTCAGTGCGTGC
AACAAGCGGGTGGGCATGTACTTAGAGGGCTTCGATCCTTTCAATCAGTCAACATTTAA
CAACGTGGTGGAGCAGAACTTTAAGAACCGCGAGAGCTACCCAGAGGACACGGTGTTCT
ACATCCCTGAAGATCACAAGCCTGGCACTTTCCCCAAAGCTCTCACCAATGGCAGTTTC
TCCCCCTCGGGGAATAACGGCTCTGTGAACTGGAGGACGGGAAGCTTCCACGGCCCTGG
CCACATCAGCCTCAGGAGGGAAAACAGTAGCGACAGCCCCAAGGAACTGAAGAGACGCA
ATTCTTCCAGCTCCATGAGCAGCCGCCTGAGCATCTACGACAACGTGCCGGGCTCCATC
CTCTACTCCAGTTCAGGGGACCTGGCGGATCTGGAGAACGAGGACATCTTCCCCGAGCT
GGACGACATCCTCTACCACGTGAAGGGGATGCAGCGGATAGTCAATCAGTGGTCGGAGA
AGTTTTCTGATGAGGGAGATTCGGACTCAGCCCTGGACTCGGTCTCTCCCTGCCCGTCC
TCTCCAAAACAGATACACCTGGATGTGGACAACGACCGAACCACACCCAGCGACCTGGA
CAGCACAGGCAACTCCCTGAATGAACCGGAAGAGCCCTCCGAGATCCCGGAAAGAAGGG
ATTCTGGGGTTGGGGCTTCCCTAACCAGGTCCAACAGGCACCGACTGAGATGGCACAGT
TTCCAGAGCTCACATCGGCCAAGCCTCAACTCTGTATCACTACAGATTAACTGCCAGTC
TGTGGCCCAGATGAACCTGCTGCAGAAATACTCACTCCTAAAGCTAACGGCCCTGCTGG
AGAAATACACACCTTCTAACAAGCATGGTTTTAGCTGGGCCGTGCCCAAGTTCATGAAG
AGGATCAAGGTTCCAGACTACAAGGACCGGAGTGTGTTTGGGGTCCCACTGACGGTCAA
CGTGCAGCGCACAGGACAACCGTTGCCTCAGAGCATCCAGCAGGCCATGCGATACCTCC
GGAACCATTGTTTGGATCAGGTTGGGCTCTTCAGAAAATCGGGGGTCAAGTCCCGGATT
CAGGCTCTGCGCCAGATGAATGAAGGTGCCATAGACTGTGTCAACTACGAAGGACAGTC
TGCTTATGACGTGGCAGACATGCTGAAGCAGTATTTTCGAGATCTTCCTGAGCCACTAA
TGACGAACAAACTCTCGGAAACCTTTCTACAGATCTACCAATATGTGCCCAAGGACCAG
CGCCTGCAGGCCATCAAGGCTGCCATCATGCTGCTGCCTGACGAGAACCGGGAGGTTCT
GCAGACCCTGCTTTATTTCCTGAGCGATGTCACAGCAGCCGTAAAAGAAAACCAGATGA
CCCCAACCAACCTGGCCGTGTGCTTAGCGCCTTCCCTCTTCCATCTCAACACCCTGAAG
AGAGAGAATTCCTCTCCCAGGGTAATGCAAAGAAAACAAAGTTTGGGCAAACCAGATCA
GAAAGATTTGAATGAAAACCTAGCTGCCACTCAAGGGCTGGCCCATATGATCGCCGAGT
GCAAGAAGCTTTTCCAGGTTCCCGAGGAAATGAGCCGATGTCGTAATTCCTATACCGAA
CAAGAGCTGAAGCCCCTCACTCTGGAAGCACTCGGGCACCTGGGTAATGATGACTCAGC
TGACTACCAACACTTCCTCCAGGACTGTGTGGATGGCCTGTTTAAAGAAGTCAAAGAGA
AGTTTAAAGGCTGGGTCAGCTACTCCACTTCGGAGCAGGCTGAGCTGTCCTATAAGAAG
GTGAGCGAAGGACCCCCTCTGAGGCTTTGGAGGTCAGTCATTGAAGTCCCTGCTGTGCC
AGAGGAAATCTTAAAGCGCCTACTTAAAGAACAGCACCTCTGGGATGTAGACCTGTTGG
ATTCAAAAGTGATCGAAATTCTGGACAGCCAAACTGAAATTTACCAGTATGTCCAAAAC
AGTATGGCACCTCATCCTGCTCGAGACTACGTTGTTTTAAGAACCTGGAGGACTAATTT
ACCCAAAGGAGCCTGTGCCCTTTTACTAACCTCTGTGGATCACGATCGCGCACCTGTGG
TGGGTGTGAGGGTTAATGTGCTCTTGTCCAGGTATTTGATTGAACCCTGTGGGCCAGGA
AAATCCAAACTCACCTACATGTGCAGAGTTGACTTAAGGGGCCACATGCCAGAATGGTA
CACAAAATCTTTTGGACATTTGTGTGCAGCTGAAGTTGTAAAGATCCGGGATTCCTTCA
GTAACCAGAACACTGAAACCAAAGACACCAAATCTAGGTGATGTACAAGTAGGAATTCG
ATATCAAGCTTATCGATAATCAACCTCTGGATTACAAAATTTGTGAAAGATTGACTGGT
ATTCTTAACTATGTTGCTCCTTTTACGCTATGTGGATACGCTGCTTTAATGCCTTTGTA
TCATGCTATTGCTTCCCGTATGGCTTTCATTTTCTCCTCCTTGTATAAATCCTGGTTGC
TGTCTCTTTATGAGGAGTTGTGGCCCGTTGTCAGGCAACGTGGCGTGGTGTGCACTGTG
TTTGCTGACGCAACCCCCACTGGTTGGGGCATTGCCACCACCTGTCAGCTCCTTTCCGG
GACTTTCGCTTTCCCCCTCCCTATTGCCACGGCGGAACTCATCGCCGCCTGCCTTGCCC
GCTGCTGGACAGGGGCTCGGCTGTTGGGCACTGACAATTCCGTGGTGTTGTCGGGGAAA
TCATCGTCCTTTCCTTGGCTGCTCGCCTATGTTGCCACCTGGATTCTGCGCGGGACGTC
CTTCTGCTACGTCCCTTCGGCCCTCAATCCAGCGGACCTTCCTTCCCGCGGCCTGCTGC
CGGCTCTGCGGCCTCTTCCGCGTCTTCGCCTTCGCCCTCAGACGAGTCGGATCTCCCTT
TGGGCCGCCTCCCCGCATCGATACCGAGCGCTGCTCGAGAGATCTACGGGTGGCATCCC
TGTGACCCCTCCCCAGTGCCTCTCCTGGCCCTGGAAGTTGCCACTCCAGTGCCCACCAG
CCTTGTCCTAATAAAATTAAGTTGCATCATTTTGTCTGACTAGGTGTCCTTCTATAATA
TTATGGGGTGGAGGGGGGTGGTATGGAGCAAGGGGCAAGTTGGGAAGACAACCTGTAGG
GCCTGCGGGGTCTATTGGGAACCAAGCTGGAGTGCAGTGGCACAATCTTGGCTCACTGC
AATCTCCGCCTCCTGGGTTCAAGCGATTCTCCTGCCTCAGCCTCCCGAGTTGTTGGGAT
TCCAGGCATGCATGACCAGGCTCAGCTAATTTTTGTTTTTTTGGTAGAGACGGGGTTTC
ACCATATTGGCCAGGCTGGTCTCCAACTCCTAATCTCAGGTGATCTACCCACCTTGGCC
TCCCAAATTGCTGGGATTACAGGCGTGAACCACTGCTCCCTTCCCTGTCCTTCTGATTT
TGTAGGTAACCACGTGCGGACCGAGCGGCCGCAGGAACCCCTAGTGATGGAGTTGGCCA
CTCCCTCTCTGCGCGCTCGCTCGCTCACTGAGGCCGGGCGACCAAAGGTCGCCCGACGC
CCGGGCTTTGCCCGGGCGGCCTCAGTGAGCGAGCGAGCGCGCAGCTGCCTGCAGGGGCG
CCTGATGCGGTATTTTCTCCTTACGCATCTGTGCGGTATTTCACACCGCATACGTCAAA
GCAACCATAGTACGCGCCCTGTAGCGGCGCATTAAGCGCGGCGGGTGTGGTGGTTACGC
GCAGCGTGACCGCTACACTTGCCAGCGCCTTAGCGCCCGCTCCTTTCGCTTTCTTCCCT
TCCTTTCTCGCCACGTTCGCCGGCTTTCCCCGTCAAGCTCTAAATCGGGGGCTCCCTTT
AGGGTTCCGATTTAGTGCTTTACGGCACCTCGACCCCAAAAAACTTGATTTGGGTGATG
GTTCACGTAGTGGGCCATCGCCCTGATAGACGGTTTTTCGCCCTTTGACGTTGGAGTCC
ACGTTCTTTAATAGTGGACTCTTGTTCCAAACTGGAACAACACTCAACTCTATCTCGGG
CTATTCTTTTGATTTATAAGGGATTTTGCCGATTTCGGTCTATTGGTTAAAAAATGAGC
TGATTTAACAAAAATTTAACGCGAATTTTAACAAAATATTAACGTTTACAATTTTATGG
TGCACTCTCAGTACAATCTGCTCTGATGCCGCATAGTTAAGCCAGCCCCGACACCCGCC
AACACCCGCTGACGCGCCCTGACGGGCTTGTCTGCTCCCGGCATCCGCTTACAGACAAG
CTGTGACCGTCTCCGGGAGCTGCATGTGTCAGAGGTTTTCACCGTCATCACCGAAACGC
GCGAGACGAAAGGGCCTCGTGATACGCCTATTTTTATAGGTTAATGTCATGATAATAAT
GGTTTCTTAGACGTCAGGTGGCACTTTTCGGGGAAATGTGCGCGGAACCCCTATTTGTT
TATTTTTCTAAATACATTCAAATATGTATCCGCTCATGAGACAATAACCCTGATAAATG
CTTCAATAATATTGAAAAAGGAAGAGTATGAGTATTCAACATTTCCGTGTCGCCCTTAT
TCCCTTTTTTGCGGCATTTTGCCTTCCTGTTTTTGCTCACCCAGAAACGCTGGTGAAAG
TAAAAGATGCTGAAGATCAGTTGGGTGCACGAGTGGGTTACATCGAACTGGATCTCAAC
AGCGGTAAGATCCTTGAGAGTTTTCGCCCCGAAGAACGTTTTCCAATGATGAGCACTTT
TAAAGTTCTGCTATGTGGCGCGGTATTATCCCGTATTGACGCCGGGCAAGAGCAACTCG
GTCGCCGCATACACTATTCTCAGAATGACTTGGTTGAGTACTCACCAGTCACAGAAAAG
CATCTTACGGATGGCATGACAGTAAGAGAATTATGCAGTGCTGCCATAACCATGAGTGA
TAACACTGCGGCCAACTTACTTCTGACAACGATCGGAGGACCGAAGGAGCTAACCGCTT
TTTTGCACAACATGGGGGATCATGTAACTCGCCTTGATCGTTGGGAACCGGAGCTGAAT
GAAGCCATACCAAACGACGAGCGTGACACCACGATGCCTGTAGCAATGGCAACAACGTT
GCGCAAACTATTAACTGGCGAACTACTTACTCTAGCTTCCCGGCAACAATTAATAGACT
GGATGGAGGCGGATAAAGTTGCAGGACCACTTCTGCGCTCGGCCCTTCCGGCTGGCTGG
TTTATTGCTGATAAATCTGGAGCCGGTGAGCGTGGGTCTCGCGGTATCATTGCAGCACT
GGGGCCAGATGGTAAGCCCTCCCGTATCGTAGTTATCTACACGACGGGGAGTCAGGCAA
CTATGGATGAACGAAATAGACAGATCGCTGAGATAGGTGCCTCACTGATTAAGCATTGG
TAACTGTCAGACCAAGTTTACTCATATATACTTTAGATTGATTTAAAACTTCATTTTTA
ATTTAAAAGGATCTAGGTGAAGATCCTTTTTGATAATCTCATGACCAAAATCCCTTAAC
GTGAGTTTTCGTTCCACTGAGCGTCAGACCCCGTAGAAAAGATCAAAGGATCTTCTTGA
GATCCTTTTTTTCTGCGCGTAATCTGCTGCTTGCAAACAAAAAAACCACCGCTACCAGC
GGTGGTTTGTTTGCCGGATCAAGAGCTACCAACTCTTTTTCCGAAGGTAACTGGCTTCA
GCAGAGCGCAGATACCAAATACTGTTCTTCTAGTGTAGCCGTAGTTAGGCCACCACTTC
AAGAACTCTGTAGCACCGCCTACATACCTCGCTCTGCTAATCCTGTTACCAGTGGCTGC
TGCCAGTGGCGATAAGTCGTGTCTTACCGGGTTGGACTCAAGACGATAGTTACCGGATA
AGGCGCAGCGGTCGGGCTGAACGGGGGGTTCGTGCACACAGCCCAGCTTGGAGCGAACG
ACCTACACCGAACTGAGATACCTACAGCGTGAGCTATGAGAAAGCGCCACGCTTCCCGA
AGGGAGAAAGGCGGACAGGTATCCGGTAAGCGGCAGGGTCGGAACAGGAGAGCGCACGA
GGGAGCTTCCAGGGGGAAACGCCTGGTATCTTTATAGTCCTGTCGGGTTTCGCCACCTC
TGACTTGAGCGTCGATTTTTGTGATGCTCGTCAGGGGGGCGGAGCCTATGGAAAAACGC
CAGCAACGCGGCCTTTTTACGGTTCCTGGCCTTTTGCTGGCCTTTTGCTCA (SEQ ID
NO: 10; the sequence of the DLC1-i1 gene is in bold italic).

In other forms, a SMN-1 gene is delivered to the subject together with the DLC1-i1 gene, for example, as part of the same or a different vector/delivery system. In an exemplary form, the SMN-1 gene is delivered to the subject as part of a separate vector from that which is used to deliver the DLC1-i1 to the same subject.

In some forms, the SMN-1 gene is delivered to the subject contained within a AAV vector configured for expression of the SMN-1 protein in a target cell under the control of the SYN promoter. An exemplary vector is a hSyn SMN-1 AAV plasmid. In some forms, the hSyn-SMN-1 AAV plasmid is constructed by pAAV-hSyn-EGFP and cDNA of SMN-1. For example, in some forms, the plasmid is hSYN-SMN-1 AAV having 5425 base pairs (bp), as depicted in FIG. 6N. In some forms, the hSyn-SMN-1 AAV includes the nucleic acid sequence:

CATGTCCTGCAGGCAGCTGCGCGCTCGCTCGCTCACTGAGGCCGCCCGGGCGTCGGGCG
ACCTTTGGTCGCCCGGCCTCAGTGAGCGAGCGAGCGCGCAGAGAGGGAGTGGCCAACTC
CATCACTAGGGGTTCCTGCGGCCGCACGCGTGTGTCTAGACTGCAGAGGGCCCTGCGTA
TGAGTGCAAGTGGGTTTTAGGACCAGGATGAGGCGGGGTGGGGGTGCCTACCTGACGAC
CGACCCCGACCCACTGGACAAGCACCCAACCCCCATTCCCCAAATTGCGCATCCCCTAT
CAGAGAGGGGGAGGGGAAACAGGATGCGGCGAGGCGCGTGCGCACTGCCAGCTTCAGCA
CCGCGGACAGTGCCTTCGCCCCCGCCTGGCGGCGCGCGCCACCGCCGCCTCAGCACTGA
AGGCGCGCTGACGTCACTCGCCGGTCCCCCGCAAACTCCCCTTCCCGGCCACCTTGGTC
GCGTCCGCGCCGCCGCCGGCCCAGCCGGACCGCACCACGCGAGGCGCGAGATAGGGGGG
CACGGGCGCGACCATCTGCGCTGCGGCGCCGGCGACTCAGCGCTGCCTCAGTCTGCGGT
GGGCAGCGGAGGAGTCGTGTCGTGCCTGAGAGCGCAGTCGAGAAGGTACCGGATCCATG
GCGATGAGCAATGGCGATGAGCAGCGGCGGCAGTGGTGGCGGCGTCCCGGAGCAGGAGG
ATTCCGTGCTGTTCCGGCGCGGCACAGGCCAGAGCGATGATTCTGACATTTGGGATGAT
ACAGCACTGATAAAAGCATATGATAAAGCTGTGGCTTCATTTAAGCATGCTCTAAAGAA
TGGTGACATTTGTGAAACTTCGGGTAAACCAAAAACCACACCTAAAAGAAAACCTGCTA
AGAAGAATAAAAGCCAAAAGAAGAATACTGCAGCTTCCTTACAACAGTGGAAAGTTGGG
GACAAATGTTCTGCCATTTGGTCAGAAGACGGTTGCATTTACCCAGCTACCATTGCTTC
AATTGATTTTAAGAGAGAAACCTGTGTTGTGGTTTACACTGGATATGGAAATAGAGAGG
AGCAAAATCTGTCCGATCTACTTTCCCCAATCTGTGAAGTAGCTAATAATATAGAACAA
AATGCTCAAGAGAATGAAAATGAAAGCCAAGTTTCAACAGATGAAAGTGAGAACTCCAG
GTCTCCTGGAAATAAATCAGATAACATCAAGCCCAAATCTGCTCCATGGAACTCTTTTC
TCCCTCCACCACCCCCCATGCCAGGGCCAAGACTGGGACCAGGAAAGCCAGGTCTAAAA
TTCAATGGCCCACCACCGCCACCGCCACCACCACCACCCCACTTACTATCATGCTGGCT
GCCTCCATTTCCTTCTGGACCACCAATAATTCCCCCACCACCTCCCATATGTCCAGATT
CTCTTGATGATGCTGATGCTTTGGGAAGTATGTTAATTTCATGGTACATGAGTGGCTAT
CATACTGGCTATTATATGGGTTTCAGACAAAATCAAAAAGAAGGAAGGTGCTCACATTC
CTTAAATTAAAAGCTTATCGATAATCAACCTCTGGATTACAAAATTTGTGAAAGATTGA
CTGGTATTCTTAACTATGTTGCTCCTTTTACGCTATGTGGATACGCTGCTTTAATGCCT
TTGTATCATGCTATTGCTTCCCGTATGGCTTTCATTTTCTCCTCCTTGTATAAATCCTG
GTTGCTGTCTCTTTATGAGGAGTTGTGGCCCGTTGTCAGGCAACGTGGCGTGGTGTGCA
CTGTGTTTGCTGACGCAACCCCCACTGGTTGGGGCATTGCCACCACCTGTCAGCTCCTT
TCCGGGACTTTCGCTTTCCCCCTCCCTATTGCCACGGCGGAACTCATCGCCGCCTGCCT
TGCCCGCTGCTGGACAGGGGCTCGGCTGTTGGGCACTGACAATTCCGTGGTGTTGTCGG
GGAAATCATCGTCCTTTCCTTGGCTGCTCGCCTATGTTGCCACCTGGATTCTGCGCGGG
ACGTCCTTCTGCTACGTCCCTTCGGCCCTCAATCCAGCGGACCTTCCTTCCCGCGGCCT
GCTGCCGGCTCTGCGGCCTCTTCCGCGTCTTCGCCTTCGCCCTCAGACGAGTCGGATCT
CCCTTTGGGCCGCCTCCCCGCATCGATACCGAGCGCTGCTCGAGAGATCTACGGGTGGC
ATCCCTGTGACCCCTCCCCAGTGCCTCTCCTGGCCCTGGAAGTTGCCACTCCAGTGCCC
ACCAGCCTTGTCCTAATAAAATTAAGTTGCATCATTTTGTCTGACTAGGTGTCCTTCTA
TAATATTATGGGGTGGAGGGGGGTGGTATGGAGCAAGGGGCAAGTTGGGAAGACAACCT
GTAGGGCCTGCGGGGTCTATTGGGAACCAAGCTGGAGTGCAGTGGCACAATCTTGGCTC
ACTGCAATCTCCGCCTCCTGGGTTCAAGCGATTCTCCTGCCTCAGCCTCCCGAGTTGTT
GGGATTCCAGGCATGCATGACCAGGCTCAGCTAATTTTTGTTTTTTTGGTAGAGACGGG
GTTTCACCATATTGGCCAGGCTGGTCTCCAACTCCTAATCTCAGGTGATCTACCCACCT
TGGCCTCCCAAATTGCTGGGATTACAGGCGTGAACCACTGCTCCCTTCCCTGTCCTTCT
GATTTTGTAGGTAACCACGTGCGGACCGAGCGGCCGCAGGAACCCCTAGTGATGGAGTT
GGCCACTCCCTCTCTGCGCGCTCGCTCGCTCACTGAGGCCGGGCGACCAAAGGTCGCCC
GACGCCCGGGCTTTGCCCGGGCGGCCTCAGTGAGCGAGCGAGCGCGCAGCTGCCTGCAG
GGGCGCCTGATGCGGTATTTTCTCCTTACGCATCTGTGCGGTATTTCACACCGCATACG
TCAAAGCAACCATAGTACGCGCCCTGTAGCGGCGCATTAAGCGCGGCGGGTGTGGTGGT
TACGCGCAGCGTGACCGCTACACTTGCCAGCGCCTTAGCGCCCGCTCCTTTCGCTTTCT
TCCCTTCCTTTCTCGCCACGTTCGCCGGCTTTCCCCGTCAAGCTCTAAATCGGGGGCTC
CCTTTAGGGTTCCGATTTAGTGCTTTACGGCACCTCGACCCCAAAAAACTTGATTTGGG
TGATGGTTCACGTAGTGGGCCATCGCCCTGATAGACGGTTTTTCGCCCTTTGACGTTGG
AGTCCACGTTCTTTAATAGTGGACTCTTGTTCCAAACTGGAACAACACTCAACTCTATC
TCGGGCTATTCTTTTGATTTATAAGGGATTTTGCCGATTTCGGTCTATTGGTTAAAAAA
TGAGCTGATTTAACAAAAATTTAACGCGAATTTTAACAAAATATTAACGTTTACAATTT
TATGGTGCACTCTCAGTACAATCTGCTCTGATGCCGCATAGTTAAGCCAGCCCCGACAC
CCGCCAACACCCGCTGACGCGCCCTGACGGGCTTGTCTGCTCCCGGCATCCGCTTACAG
ACAAGCTGTGACCGTCTCCGGGAGCTGCATGTGTCAGAGGTTTTCACCGTCATCACCGA
AACGCGCGAGACGAAAGGGCCTCGTGATACGCCTATTTTTATAGGTTAATGTCATGATA
ATAATGGTTTCTTAGACGTCAGGTGGCACTTTTCGGGGAAATGTGCGCGGAACCCCTAT
TTGTTTATTTTTCTAAATACATTCAAATATGTATCCGCTCATGAGACAATAACCCTGAT
AAATGCTTCAATAATATTGAAAAAGGAAGAGTATGAGTATTCAACATTTCCGTGTCGCC
CTTATTCCCTTTTTTGCGGCATTTTGCCTTCCTGTTTTTGCTCACCCAGAAACGCTGGT
GAAAGTAAAAGATGCTGAAGATCAGTTGGGTGCACGAGTGGGTTACATCGAACTGGATC
TCAACAGCGGTAAGATCCTTGAGAGTTTTCGCCCCGAAGAACGTTTTCCAATGATGAGC
ACTTTTAAAGTTCTGCTATGTGGCGCGGTATTATCCCGTATTGACGCCGGGCAAGAGCA
ACTCGGTCGCCGCATACACTATTCTCAGAATGACTTGGTTGAGTACTCACCAGTCACAG
AAAAGCATCTTACGGATGGCATGACAGTAAGAGAATTATGCAGTGCTGCCATAACCATG
AGTGATAACACTGCGGCCAACTTACTTCTGACAACGATCGGAGGACCGAAGGAGCTAAC
CGCTTTTTTGCACAACATGGGGGATCATGTAACTCGCCTTGATCGTTGGGAACCGGAGC
TGAATGAAGCCATACCAAACGACGAGCGTGACACCACGATGCCTGTAGCAATGGCAACA
ACGTTGCGCAAACTATTAACTGGCGAACTACTTACTCTAGCTTCCCGGCAACAATTAAT
AGACTGGATGGAGGCGGATAAAGTTGCAGGACCACTTCTGCGCTCGGCCCTTCCGGCTG
GCTGGTTTATTGCTGATAAATCTGGAGCCGGTGAGCGTGGGTCTCGCGGTATCATTGCA
GCACTGGGGCCAGATGGTAAGCCCTCCCGTATCGTAGTTATCTACACGACGGGGAGTCA
GGCAACTATGGATGAACGAAATAGACAGATCGCTGAGATAGGTGCCTCACTGATTAAGC
ATTGGTAACTGTCAGACCAAGTTTACTCATATATACTTTAGATTGATTTAAAACTTCAT
TTTTAATTTAAAAGGATCTAGGTGAAGATCCTTTTTGATAATCTCATGACCAAAATCCC
TTAACGTGAGTTTTCGTTCCACTGAGCGTCAGACCCCGTAGAAAAGATCAAAGGATCTT
CTTGAGATCCTTTTTTTCTGCGCGTAATCTGCTGCTTGCAAACAAAAAAACCACCGCTA
CCAGCGGTGGTTTGTTTGCCGGATCAAGAGCTACCAACTCTTTTTCCGAAGGTAACTGG
CTTCAGCAGAGCGCAGATACCAAATACTGTTCTTCTAGTGTAGCCGTAGTTAGGCCACC
ACTTCAAGAACTCTGTAGCACCGCCTACATACCTCGCTCTGCTAATCCTGTTACCAGTG
GCTGCTGCCAGTGGCGATAAGTCGTGTCTTACCGGGTTGGACTCAAGACGATAGTTACC
GGATAAGGCGCAGCGGTCGGGCTGAACGGGGGGTTCGTGCACACAGCCCAGCTTGGAGC
GAACGACCTACACCGAACTGAGATACCTACAGCGTGAGCTATGAGAAAGCGCCACGCTT
CCCGAAGGGAGAAAGGCGGACAGGTATCCGGTAAGCGGCAGGGTCGGAACAGGAGAGCG
CACGAGGGAGCTTCCAGGGGGAAACGCCTGGTATCTTTATAGTCCTGTCGGGTTTCGCC
ACCTCTGACTTGAGCGTCGATTTTTGTGATGCTCGTCAGGGGGGCGGAGCCTATGGAAA
AACGCCAGCAACGCGGCCTTTTTACGGTTCCTGGCCTTTTGCTGGCCTTTTGCTCA (SEQ
ID NO: 11; the sequence of the SMN-1 gene is in bold italic).

### III. Methods of Treatment

Methods for using rAAV-DLC1-i1, optionally including rAAV-SMN1, are provided. Typically, the methods treat or prevent Spinal muscular atrophy (SMA) in a subject in need thereof. Therefore, methods of treating or preventing one or more symptoms of SMA in a subject in need thereof are described. In some forms, the methods including a step of selecting a subject who is likely to benefit from treatment with the described compositions.

### A. Methods of Treating Spinal Muscular Atrophy (SMA)

Methods for the treatment, amelioration or prevention of Spinal muscular atrophy (SMA) in a subject are provided.

As described in the Examples, the administration of AAV-SYN-DLC1-i1 via intra-cerebraventricular (ICV) in vivo gave rise to a significant increase of lifespan, shorter righting time, higher ambulation scores and longer four limbs hanging time compared to controls in experimental animal models.

As also described in the Examples, the lifespans of middle dosage (5×10¹⁰ vg) AAV-SYN-DLC1-i1 treated animals was a mean of 42.7 days, as compared with only 30.2 days for those treated with commercially-available drug (ZOLGENSMA™), whilst the lifespan of high dosage (1×10¹¹ vg) AAV-SYN-DLC1-i1 treated animals was a mean of 43.7 days. As compared with only 17 days for those treated with commercially-available drug (ZOLGENSMA™), which is significantly lower than the lifespan of the same dosage of DLC1-i1 treated mice.

Therefore, behavior tests showed that AAV-SYN-DLC1-i1 is more effective than AAV-SYN-SMN1 in improving mice locomotion and muscle strength, especially the significant longer hanging time in all three dosages of DLC1-i1 compared to SMN1.

Typically, the methods deliver compositions including AAV-SYN-DLC1-i1 to the brain tissue or CNS tissue via intravenous (iv), intra-cerebraventricular (ICV), or intracranial administration to a subject. The composition is administered in an amount sufficient to induce a therapeutic effect in the subject. Exemplary methods include administering to the spinal cord and/or cistema magna of the subject an effective amount of rAAV viral particles including a vector encoding a DLC1-i1 gene or gene expression product. In some forms, the methods treat a human with SMA, e.g., a pediatric subject, or an adult, to improve the pathologies associated with spinal muscular atrophy. In some forms, the viral particle includes an AAV serotype 9 capsid (AAV9 capsid) and AAV2 inverted terminal repeats. In some forms, the viral particle includes a recombinant self-complementing vector genome for efficient expression of the transgene in motor neurons upon viral transduction.

In some forms, the methods administer one or more additional active agents to the subject before, at the same time, or after the administration of the composition of AAV-SYN-DLC1-i1. The co-administered agent can be in the same, or different composition, and can be administered at the same or a distant site.

In some forms, the methods repeat the step of administering the composition of AAV-SYN-DLC1-i1 composition to the CNS and/or brain of the subject. Typically, the second administration is carried out at a time of one, two, three, four, five, six, seven, eight, nine, or ten days or weeks after the first administration.

In some forms, the methods deliver a heterologous transgene encoding a primate SMN to a motor neuron in a subject, the method including administering to the spinal cord or cisterna magna of the subject, an effective amount of rAAV viral particles including vector encoding the primate DLC1-i1 transgene. Typically, the administration delivers the transgene product to the motor neuron's cellular environment, where the SMN mediates a beneficial effect on the cell and surrounding cells. In some forms the motor neurons are in the spinal cord of the subject. In some forms, the methods deliver a trans gene expressing human SMN to motor neurons that are in the spinal cord of the subject.

In some forms, the methods include a combination of treatments. The combination therapies and treatment regimes disclosed herein typically include treatment of a disease or symptom thereof, or a method for achieving a desired physiological change, including administering to an animal, such as a mammal, especially a human being, an effective amount of a DLC1-i1, alone or in combination with an effective amount of SMN1 to treat the disease or symptom thereof, or to produce the physiological change. In some forms, the agents or components for delivery of DLC1-i1 in combination with an effective amount of SMN1 are administered together, such as part of the same composition, or administered separately and independently at the same time or at different times (i.e., administration of the DLC1-i1 gene and the SMN1 gene is separated by a finite period of time from each other). Therefore, the term “combination” or “combined” is used to refer to either concomitant, simultaneous, or sequential administration of the DLC1-i1 gene and the SMN1 gene. The combinations can be administered either concomitantly (e.g., as an admixture), separately but simultaneously (e.g., via separate intravenous lines into the same subject; one agent is given orally while the other agent is given by infusion or injection, etc.), or sequentially (e.g., one agent is given first followed by the second).

When used for treating SMA, the amount of DLC1-i1 gene present in a pharmaceutical dosage unit, or otherwise administered to a subject can be the amount effective to reduce one or more symptoms or physiological effects of the SMA when administered in combination with a SMN1 gene. Likewise, the amount of the SMN1 gene present in a pharmaceutical dosage unit, or otherwise administered to a subject can be the amount effective to reduce one or more symptoms or physiological effects of the SMA when administered in combination with a DLC1-i1 gene. Therefore, in some forms the amount of the active agents is effective to reduce, slow or halt one or more symptoms of SMA in the treated subject. In some forms, the amount of the active agents is effective to alter a measurable biochemical or physiological marker. In some forms, the active agents are administered in an effective amount to reduce or prevent SMA development or progression.

In preferred forms, administration of the DLC1-i1 gene and the SMN1 gene achieves a result greater than when the DLC1-i1 gene and the SMN1 gene are administered alone or in isolation (i.e., the result achieved by the combination is more than additive of the results achieved by the individual components alone). In some forms, the effective amount of one or both agents used in combination is lower than the effective amount of each agent when administered separately. In some embodiments, the amount of one or both agents when used in the combination therapy is sub-therapeutic when used alone.

The effect of the combination therapy, or individual agents thereof can depend on the disease or condition to be treated or progression thereof. For example, as illustrated in the Examples below, an agent such as the described DLC1-i1 gene therapy alone or in combination with the SMN1 gene therapy can be used a first or second line therapy for treatment of SMA. Accordingly, in some embodiments, the effect of the combination on SMA can compared to the effect of the individual agents alone on SMA. In some forms, the combination is improved over the individual components alone. In some forms, although the therapeutic effect of the combination is similar to the individual components, the duration of efficacy of the treatment is longer. This allows the disclosed combination therapies to be administered in combination with or as an alternative to other therapy, a first line therapy, or a second line or subsequent therapy.

A treatment regimen of the combination therapy can include one or multiple administrations of the DLC1-i1 gene and/or the SMN1 gene. In some forms, the DLC1-i1 gene is administered prior to the first administration of the SMN1 gene. In other embodiments, the SMN1 gene is administered prior to the first administration of the DLC1-i1 gene.

The DLC1-i1 gene therapy gene can be administered at least 1, 2, 3, 5, 10, 15, 20, 24 or 30 hours or days prior to or after administering of the SMN1 gene therapy. Alternatively, the SMN1 gene can be administered at least 1, 2, 3, 5, 10, 15, 20, 24 or 30 hours or days prior to or after administering of the DLC1-i1 gene.

Dosage regimens or cycles of the agents can be completely, or partially overlapping, or can be sequential. For example, in some forms, all such administration(s) of the DLC1-i1 gene occur before or after administration of the SMN1 gene. Alternatively, administration of one or more doses of the DLC1-i1 gene can be temporally staggered with the administration of the SMN1 gene to form a uniform or non-uniform course of treatment whereby one or more doses of the DLC1-i1 gene, followed by one or more doses of the SMN1 gene, followed by one or more doses of the DLC1-i1 gene; or one or more doses of the SMN1 gene are administered, followed by one or more doses of the DLC1-i1 gene, followed by one or more doses of the SMN1 gene etc., all according to whatever schedule is selected or desired by the researcher or clinician administering the therapy.

An effective amount of each of the agents can be administered as a single unit dosage (e.g., as dosage unit), or sub-therapeutic doses that are administered over a finite time interval. Such unit doses may be administered on a daily basis for a finite time period, such as up to 3 days, or up to 5 days, or up to 7 days, or up to 10 days, or up to 15 days or up to 20 days or up to 25 days, are all specifically contemplated.

### 1. Symptoms to be Treated

In preferred forms, the methods provide protective therapeutic efficacy by reducing or preventing one or more of the symptoms of SMA. For example, in some forms, the methods ameliorate a symptom of SMA, by administering to the spinal cord and/or cistema magna of a subject an effective amount of rAAV viral particles including a vector encoding a primate DLC1-i1.

Symptoms that can be ameliorated, treated or prevented by the methods include floppy or weak arms and legs; movement problems, such as difficulty sitting up, crawling or walking; twitching or shaking muscles (tremors); bone and joint problems, such as an unusually curved spine (scoliosis); swallowing problems and breathing difficulties, muscle wasting, paralysis, bulbar and respiratory dysfunction, motor neuron cell loss and neuromuscular junction pathology.

Amelioration of the symptoms of SMA can be measured by improved motor muscle action potential, achieved milestones, decreased dependency on ventilation, increased quality of life and longevity. For example, improvement can be measured by less dependence on ventilation and cough assistance machines, or the use of feeding tubes. Improvement can also be measured by gross motor functions such as sitting unaided, head control and the ability to walk. Increases in motor unit number estimation (MUNE), improvement in compound motor action potential (CMAP), increase in Hammersmith functional motor score (HFMS), improvement in pulmonary functional tests (FVC), and improvement of gross muscle physiology using MRI imaging, alone or in combination are indicative of therapeutic efficacy. Milestones can be measured with respect to the subject before the treatment of the invention, in comparison with non-treated control subjects, or in comparison with historical records.

### B. Individuals to be Treated

A subject in need of treatment is a subject having or at risk of having spinal muscular atrophy (SMA). Methods of assessing and detecting SMA in a subject are known by those of ordinary skill in the art. Methods of assessing the risk of SMA in a subject are known by those of ordinary skill in the art. In some forms, the subject who is at an elevated risk of an SMA may be an apparently healthy subject. An apparently healthy subject is a subject who has no signs or symptoms of disease.

SMA may be caused by mutations in the DLC1 gene that encodes the DLC1 proteins. In some forms, the methods a treat humans with a mutation in the SMN1 gene and/or in the SMN protein. In some forms, the expression of functional DLC1 in motor neurons is deficient compared to the expression of DLC1 in motor neurons of a human without SMA. In some forms, the expression of DLC1 is deficient in motor neurons of the brain and/or spinal cord.

There are three types of SMA in terms of disease severity which are related to the expression of SMN2 in the subject. Type I SMA is characterized by early onset (<6 months of age, with death typically <3 years of age) and a SMN2 copy number of 1-2. Type I SMA subjects never achieve the ability to sit and have respiratory and bulbar dysfunction. Type II SMA onset is typically between 6 and 18 months of age, with death typically at <30-40 years of age. Type II SMA is typically associated with a SMN2 copy number of 2-3. Type II subjects never achieve the ability to walk and eventually succumb to respiratory dysfunction. Type III SMA onset is typically at >18 months of age with death at >60 years of age. Type III SMA is associated with a SMN2 copy number of 3-4. Type III patients are often confined to a wheelchair by teenage and have no respiratory dysfunction.

Therefore, in some forms, the methods treat a subject with Type I SMA. In some forms, the methods treat a subject with Type II SMA. In some forms, the methods treat a subject with Type III SMA. In some forms, the methods treat a subject with Type I or Type II SMA. In some forms, the methods treat a subject with Type II or Type III SMA. In some forms, the methods treat a subject who has an snm2 copy number of 1-2, 2-3 or 3-4.

In some forms, the methods treat a pediatric human subject with SMA. In some forms, the pediatric human subject is less than any one of 2 months of age, 3 months of age, 4 months of age, 5 months of age, 6 months of age, 7 months of age, 8 months of age, 9 months of age, 10 months of age, 11 months of age, 12 months of age, 13 months of age, 14 months of age, 15 months of age, 16 months of age, 17 months of age, 18 months of age, 1 year of age, 2 years of age, 3 years of age, 4 years of age, 5 years of age, 6 years of age, 7 years of age, 8 years of age, 9 years of age, 10 years of age, 11 years of age, 12 years of age, 13 years of age, 14 years of age, 15 years of age, 16 years of age, 17 years of age, 18 years of age. In some forms, the subject is greater than 18 years of age.

In some forms, the subject has been previously treated for SMA. In some forms, the subject is currently being treated for SMA. For example, in certain forms, the subject is undergoing, has previously undergone, or will undergo treatment for SMA with one or more drugs. Exemplary drugs include those approved for treatment of SMA, such as Nusinersen or Onasemnogene abeparvovec-xioi (ZOLGENSMA™). Therefore, in some forms, the methods are carried out in conjunction with other treatment regimens involving one or more other drugs. In an exemplary form, a subject is treated by administration of ZOLGENSMA™ at the same time, before or after being treated with a composition of AAV-SYN-DLC1-i1.

### C. Dosing and Effective Amounts

In some forms, the administration to the spinal cord and/or cisterna magna of an effective amount of rAAV viral particles including a vector encoding a DLC1-i1 transgene transduces motor neurons at the vertebrate section site of administration. In some forms, the dosage includes an amount effective to transduce more than about any of 5%, 10%, 15%, 20%, 25%, 30%, 35%, 40%, 45%, 50%, 55%, 60%, 65%, 70%, 75% or 100% of motor neurons. In some forms, more than about any of 5%, 10%, 15%, 20%, 25%, 30%, 35%, 40%, 45%, 50%, 55%, 60%, 65%, 70%, 75% or 100% of motor neurons throughout the spinal cord are transduced (e.g., throughout the lumbar, thoracic, and cervical regions).

Methods to identify motor neurons transduced by AAV expressing DLC1-i1 are known in the art; for example, immunohistochemistry can be used to detect expression of DLC1-i1 using an anti-DLC1 antibody and motor neurons can be identified using an anti-choline acetyl transferase (ChAT) antibody. In some forms, more than about any of 5%, 10%, 15%, 20%, 25%, 30%, 35%, 40%, 45%, 50%, 55%, 60%, 65%, 70%, 75% or 100% of motor neurons are transduced in an animal model of SMA, e.g., a mouse model of SMA, or in a nonhuman primate.

In some forms, the transduction of motor neurons following administration to the spinal cord and/or cisterna magna of a subject an effective amount of AAV viral particles including a vector encoding a DLC1-i1 transgene results in the expression of DLC1-i1 to provide benefit to a subject with SMA. In some forms, administration to the spinal cord and/or cisterna magna of an AAV viral particle including a transgene encoding a DLC1-i1 results in expression of at least about any of 20%, 25%, 30%, 35%, 40%, 45%, 50%, 55%, 60%, 65%, 70%, 75%, 80%, 85%, 90%, 95% or 100% of levels of DLC1-i1 expression in a normal individual. Methods to measure expression of DLC1-i1 are known in the art; for example, using an anti-DLC1-i1 antibody.

In some forms, at least 1×10¹² genome copies are administered to the subject.

In some forms, the methods include administration to the spinal cord and/or cisterna magna of a subject an effective amount of AAV viral particles including a vector encoding a primate DLC1-i1. In some forms, the viral titer of the composition is at least about any of 5×10¹², 6×10¹², 7×10¹², 8×10¹², 9×10¹², 1×10¹³, 1.1×10¹³, 1.5×10¹³, 2×10¹³, 2.5×10¹³, 3×10¹³, 5×10¹³ or 1×10¹⁴ genome copies/mL. In some forms, the dose of viral particles administered to the subject is about any of 1×10¹¹ to 5×10¹¹, 5×10¹¹ to 1×10¹², or 1×10¹² to 5×10¹², 5×10¹² to 1×10¹³, or 1×10¹³ to 5×10¹⁴ genome copies/kg of body weight. In some forms, the methods include administration to the spinal cord and/or cisterna magna of a human an effective amount of AAV viral particles including a vector encoding a DLC1-i1 to a subject. In some forms, the total amount of viral particles administered to the subject is at least about any of 1×10¹², 2×10¹², 3×10¹², 4×10¹², 5×10¹², 6×10¹², 7×10¹², 8×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, or 1×10¹⁵, 2×10¹⁵ genome copies.

### D. Route of Administration

In some forms, the methods include administration to the spinal cord and/or cisterna magna of a subject an effective amount of AAV viral particles including a vector encoding a DLC1-i1 for treating a subject, e.g., a human, with SMA. In some forms, the composition is injected to one or more intrathecal spaces in the spinal cord and or in the cisterna magna to allow expression of DLC1-i1 in motor neurons. In some forms, the composition is injected into the cisterna magna. In some forms, the composition is injected into the subarachnoid space of the spinal column at one or more locations in the cervical, thoracic, lumbar or sacral regions of the spinal cord.

In some forms, the composition is injected into any one of one, two, three, four, five, six, seven, eight, nine, ten or more than ten locations in the subarachnoid space of the spinal cord. In some forms, the composition is injected to the cisterna magna and the spinal cord. In some forms, the composition is injected to the cisterna magna and into any one of one, two, three, four, five, six, seven, eight, nine, ten or more than ten locations in the subarachnoid space of the spinal cord. In some forms, the composition is injected into the subarachnoid space of the spinal cord using a catheter or other devices for intrathecal injection known in the art. In some forms the rAAV viral particles are administered to more than one location simultaneously or sequentially. In some form, multiple injections of rAAV viral particles are no more than one hour, two hours, three hours, four hours, five hours, six hours, nine hours, twelve hours or 24 hours apart.

### IV. Kits for Administration

In one form a kit is provided including one or more containers filled with one or more of the following components: A recombinant AAV viral particle including a vector encoding a Deleted in Liver Cancer 1-isoform 1 (DLC1-i1) transgene, or the expression product thereof, a composition for administration to a subject in vivo, and apparatus for mixing and administering the reagents to a subject. In some forms, the kits additionally contain a device for injecting into the spine. Associated with such a kit can be instructions on how to use the kit and optionally a notice in the form prescribed by a governmental agency regulating the manufacture, use or sale of pharmaceuticals or biological products, which notice reflects approval by the agency of manufacture, use or sale for human administration.

The disclosure will be further understood by reference to the following paragraphs:

1. A recombinant AAV viral particle including a vector encoding a Deleted in Liver Cancer 1-isoform 1 (DLC1-i1) transgene, or the expression product thereof.
2. The recombinant AAV viral particle of paragraph 1, wherein the DLC1-i1 transgene is operably linked to a promoter selected from the group including the human synapsin 1 gene promoter (SYN), human β-glucuronidase promoter and a cytomegalovirus enhancer linked to a chicken β-actin promoter.
3. The recombinant AAV viral particle of paragraph 1, wherein the DLC1-i1 transgene is operably linked to the human synapsin 1 gene promoter (SYN).
4. The recombinant AAV viral particle of any one of paragraphs 1-3, wherein the vector includes the nucleic acid sequence of SEQ ID NO: 1, and/or sequence of SEQ ID NO: 2, and/or a polynucleotide encoding the polypeptide having an amino acid sequence of SEQ ID NO:3.
5. The recombinant AAV viral particle of any one of paragraphs 1-4, wherein the AAV viral particle includes an AAV1, AAV2, AAV3, AAV4, AAV5, AAV6, AAV7, AAVS, AAVrh8, AAV9, AAV10, AAVrh10, AAV11, or AAV12 serotype capsid.
6. The recombinant AAV viral particle of any one of paragraphs 1-5, wherein the rAAV viral particle includes an AAV9 serotype capsid.
7. The recombinant AAV viral particle of any one of paragraphs 1-6, wherein the AAV viral particle includes an AAV serotype capsid from Clades A-F.
8. The recombinant AAV viral particle of any one of paragraphs 1-7, wherein, wherein the vector includes AAV1, AAV2, AAV3, AAV4, AAV5, AAV6, AAV7, AAVS, AAVrh8, AAV9, AAV10, AAVrh10, AAV11, or AAV12 serotype inverted terminal repeats (ITRs).
9. The recombinant AAV viral particle of any one of paragraphs 1-4, wherein the vector includes AAV serotype 2 ITRs.
10. The recombinant AAV viral particle of any one of paragraphs 1-9, further including a survival motor neuron 1 (SMN1) transgene, wherein the SMN1 transgene is within the same or different vector as the DLC1-i1 transgene.
11. The recombinant AAV viral particle of paragraph 10, wherein the SMN1 transgene is operably linked to the same or different promoter as the DLC1-i1 transgene.
12. The recombinant AAV viral particle of paragraph 10 or 11, wherein the recombinant AAV viral particle of paragraph 1, wherein the SMN1 transgene is operably linked to the human synapsin 1 gene promoter (SYN).
13. A composition for administration to a subject in vivo, including
(a) the recombinant AAV viral particle of any one of paragraphs 1-12; and
(b) a pharmaceutically acceptable excipient for administration.
   14. The composition of paragraph 13, further including
(c) a recombinant AAV viral particle including a survival motor neuron 1 (SMN1) transgene, optionally wherein the SMN1 transgene is operably linked to a synapsin 1 gene promoter (SYN).
   15. The composition of paragraph 13 or 14, including the recombinant AAV viral particles in an amount of between about 2.5×10¹² genome copies and about 5×10¹³ genome copies.
   16. The composition of any one of paragraphs 13 to 15, including the recombinant AAV viral particle in an amount of least 5×10¹³ genome copies per kg body weight of the subject.
   17. A method for treating spinal muscular atrophy (SMA) in a subject, including administering to the subject a first composition including the composition of any one of paragraphs 13-16.
   18. The method of paragraph 17, wherein the method ameliorates or minimizes one or more symptoms of SMA in the subject, wherein the one or more symptom is selected from the group including muscle wasting, inability to achieve motor milestones, inability to sit, inability to walk, paralysis, respiratory dysfunction, bulbar dysfunction, motor neuron cell loss and neuromuscular junction pathology.
   19. The method of paragraph 17 or 18, wherein the composition includes at least 1×10¹² genome copies of recombinant AAV including a DLC1-i1 transgene.
   20. The method of any one of paragraphs 17 to 19, wherein the methods administer at least 3.5×10¹¹ genome copies per kg body weight of recombinant AAV viral particles including a DLC1-i1 transgene to the subject.
   21. The method of any one of paragraphs 17 to 20, wherein at least 10-30% of motor neurons in the lumbar, thoracic and cervical regions of the spinal cord of the subject are transduced by the recombinant AAV viral particles.
   22. The method of any one of paragraphs 17 to 21, wherein at least 30% of wild type level of DLC1-i1 is generated throughout the spinal cord.
   23. The method of any one of paragraphs 17 to 22, wherein the composition is administered to the subject via intravenous injection, or via direct injection into the spinal cord, or via intrathecal injection, or via intracisternal injection.
   24. The method of any one of paragraphs 17 to 23, wherein the composition is administered to more than one location of the spinal cord or cisterna magna.
   25. The method of paragraph 24, wherein the composition is administered to more than one location of the spinal cord.
   26. The method of any one of paragraphs 17 to 23, wherein the composition is administered to one or more of a lumbar subarachnoid space, thoracic subarachnoid space and a cervical subarachnoid space of the spinal cord.
   27. The method of paragraph 26, wherein the composition is administered to the cisterna magna.
   28. The method of any one of paragraphs 17-27, further including administering to the subject a second composition including a recombinant AAV viral particle including a survival motor neuron 1 (SMN1) transgene, optionally wherein the SMN1 transgene is operably linked to a synapsin 1 gene promoter (SYN).
   29. The method of paragraph 28, wherein the SMN1 transgene includes the polynucleotide of SEQ ID NO:4 and/or encodes a polypeptide having the amino acid sequence of SEQ ID NO:5.
   30. The method of paragraph 28 or 29, wherein the second composition is administered to the subject at the same time as, before or after the first composition.
   31. The method of any one of paragraphs 28 to 30, wherein the therapeutic effect of the administering the first and second compositions to the subject is greater than the additive effects of administering the first composition alone or the second composition alone.
   32. The method of any one of paragraphs 28 to 31, wherein the methods administer at least 3.5×10¹¹ genome copies per kg body weight of recombinant AAV viral particles including a SMN1 transgene to the subject.
   33. The method of any one of paragraphs 28 to 32, wherein the subject is a pediatric subject.
   34. The method of any one of paragraphs 28 to 33, wherein the subject is a young adult.
   35. The method of any one of paragraphs 28 to 34, wherein the subject has spinal muscular atrophy, optionally wherein the subject has a mutation in the endogenous DLC1-i1 gene, and/or the SMN-1 gene.
   36. The method of paragraph 35, wherein the subject has a partial deletion of the endogenous DLC1-i1 gene, and/or the SMN1 gene.
   37. The method of paragraph 35, wherein the subject has a complete deletion of the endogenous DLC1-i1 gene, and/or the SMN1 gene.
   38. The method of any one of paragraphs 35 to 37, wherein expression of the mutant DLC1-i1 gene, and/or the SMN-1 gene in spinal cord or brain of the subject is deficient compared to expression of DLC1-i1, and/or SMN-1 in a subject with a wild-type DLC1-i1 gene, and/or SMN-1 gene.
   39. A recombinant viral particle including the nucleic acid sequence of SEQ ID NO:10 or 11.
   40. The recombinant viral particle of paragraph 39, including the nucleic acid sequence of SEQ ID NO: 10.
   41. A cell including the recombinant viral particle of paragraph 40.
   42. The cell of paragraph 41, further including the nucleic acid sequence of SEQ ID NO:11.

The present invention is further illustrated by the following non-limiting examples. The entire contents of all of the references (including literature references, issued patents, published patent applications, and co-pending patent applications) cited throughout this application are hereby expressly incorporated by reference.

### EXAMPLES

### Example 1: Expression of DLC1-i1 in Human and Rats

DLC1-i1 is the predominant isoform highly expressed during MN development. Because human DLC1 has five isoforms (i1-i5) and there is no study which DLC1 isoforms is/are predominantly expressed in motor neurons, different primers were designed specific for each individual isoform of DLC1. The expression of DLC1-i1, i2, i3, i4 and i5 were examined during different stages of MN differentiation including hESCs, EB, neural spheres (NSP) after neural induction, motor neuron progenitors (MNPs), MNs at 7 days post-differentiation of MNPs and MNs 21 days post-differentiation of MNPs as illustrated in FIG. 1A. qRT-PCR showed DLC1-i1 expression is significantly increased during motor neuron differentiation including MNPs, 7 D, and 21 D post-differentiation (FIG. 1B). However, the other isoforms of DLC1 (isoforms 2, 3, 4, 5) did not show elevated expression except DLC1-i3 which showed a slightly increase in expression at 7 D and 21 D post-differentiation. Therefore, DLC1-i1 is the predominant isoform highly expressed during MNs differentiation. Furthermore, it was observed that expression of DLC1-i1 has a similar pattern with the MN marker ISLET1 as iPSCs differentiated into MNs, indicating the DLC1-i1 may be involved in MN development (FIG. 1C). To examine whether DLC1-i1 expression is conserved between humans and rodents, adult rats' spinal cords were collected and the expression of DLC1-i1 was examined by immunofluorescence staining. DLC1-i1 is co-expressed with ISLET1/2 which marks soma of MNs in grey matter and in axons of MNs in white matter.

Altogether, DLC1-i1 is highly expressed in the nucleus and cytosol of human MNs and rodents MNs.

### Example 2: SMA Patients' iPSCs-Derived MNs Showed Reduced Level of DLC1-i1 Expression

Reprogramming of Healthy Individual- and SMA Patients-Derived Urine Cells into iPSC's (UiPSC's)

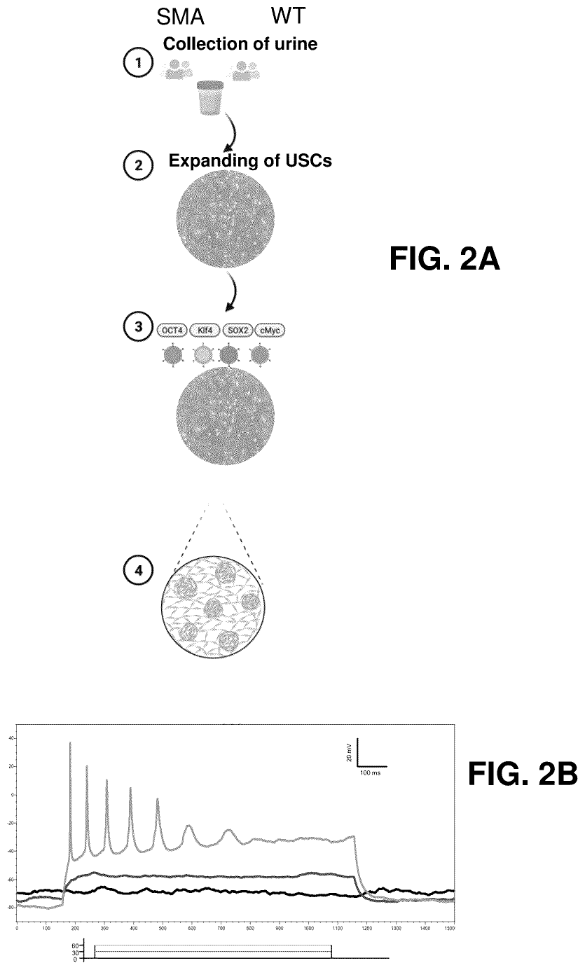

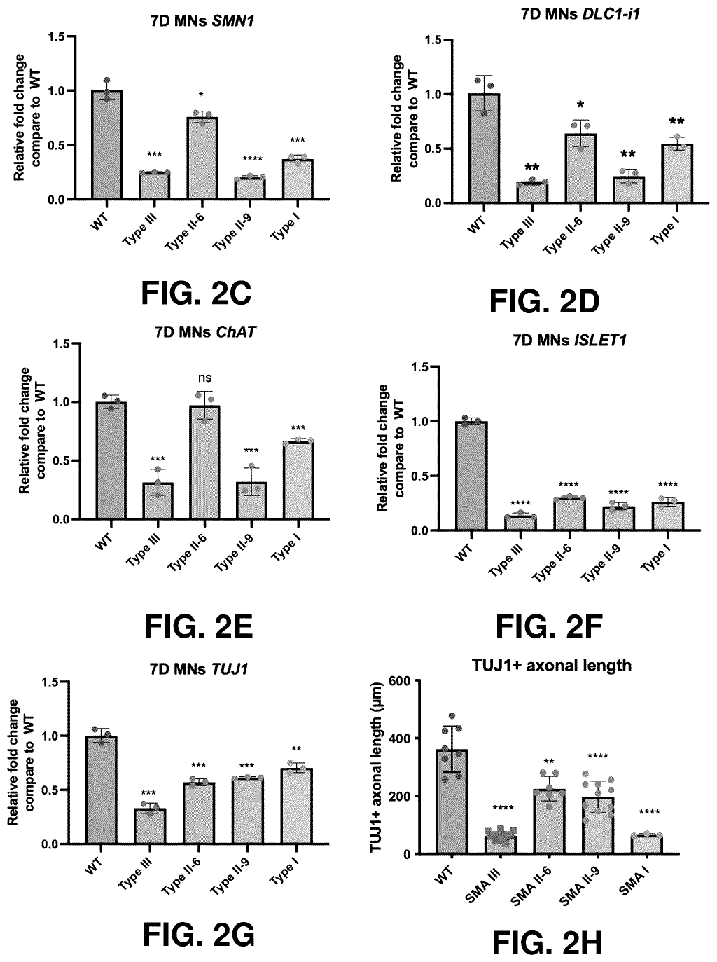

To examine the expression levels of DLC1-i1 in SMA patients' motor neurons, urine samples were first collected from SMA patients (type I×1, type II-6, type II-9 and type III×1) with the help of Dr. Chan Hoi Shan Sophelia in the Department of Pediatrics and Adolescent Medicine, Queen Mary Hospital. After a week of culture, small colonies were obtained of urine stem cells which can be reprogrammed into iPSCs (UiPSCs) by dox-inducible lentiviruses encoding OCT4, KLF4, SOX2 and c-MYC (FIG. 2A). Wild-type (WT) UiPSCs were also generated from healthy individual-derived urine stem cells as control. Both WT UiPSCs and SMA patients' UiPSCs express pluripotency markers (OCT3/4, NANOG, and SOX2). They can differentiate into MN progenitors (MNPs) expressing SOX2 and OLIG2 and post-mitotic MNs expressing ISLET1/2, TUJ1, and MAP2. After 60 days of MN differentiation, by whole-cell patch-clamp assay, we detected robust action potential with regular trains of spikes in the MNs under steps current stimulation FIG. 2B), demonstrating that urine-derived MNs are functional and electrically active.

Reduced Expression of DLC1-i1 in MNs from SMA UiPSC's

After 7 days differentiation of MNPs, immunofluorescence staining showed decreased expressions of DLC1-i1, ISLET1/2 and TUJ1; no change of OLIG2 and SOX2; increased percentage of cleaved CASPASE3, in SMA patients' MNs compared to WT. qPCR analysis further confirmed reduction of SMN1 and DLC1-i1 mRNAs in all types of SMA patients' MNs compared to WT (FIGS. 2C-2D). UiPSCs from SMA types I, II (6) and II (9) gave rise to less than 20% ISLET1/2+ MNs whereas type III UiPSCs differentiated into ˜40% ISLET1/2+ MNs compared to 50% WT UiPSCs-derived MNs. Consistently, there was a marked reduction of ISLET1 and ChAT mRNAs in SMA patients-derived MNs compared to WT (FIGS. 2E-2F). qPCR analysis further showed decrease expression of pan-neuronal marker TUJ1 in SMA patients-derived neuronal population with marked reduction in axonal length (FIGS. 2G-2H). More cells were detected positively marked by cleaved caspase-3 (cCaspase3) from SMA-derived MNs whereas cCaspase3 expression was barely detectable in WT cells. In contrast, expression of OLIG2 and SOX2 remained unaltered. Altogether, these data show that SMA patients UiPSCs exhibit defects in MN differentiation that was associated with reduced level of DLC1-i1 expression.

### Example 3: DLC1 is Required for Motor Neuron Differentiation

### KD of DLC1-i1 LED to Fewer Motor Neuron Formation and Shorter Axons

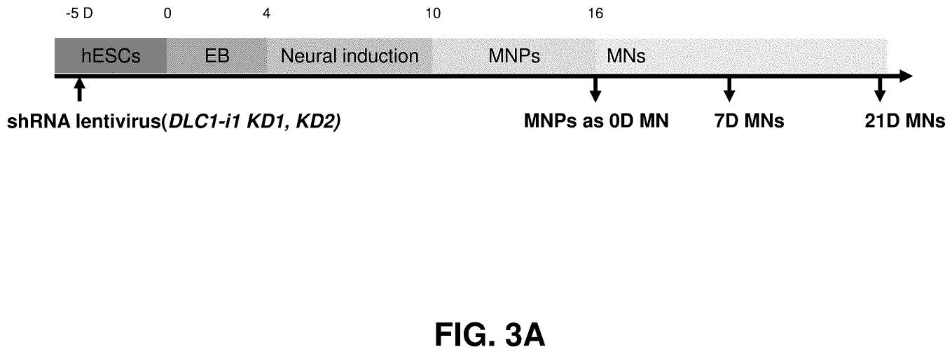

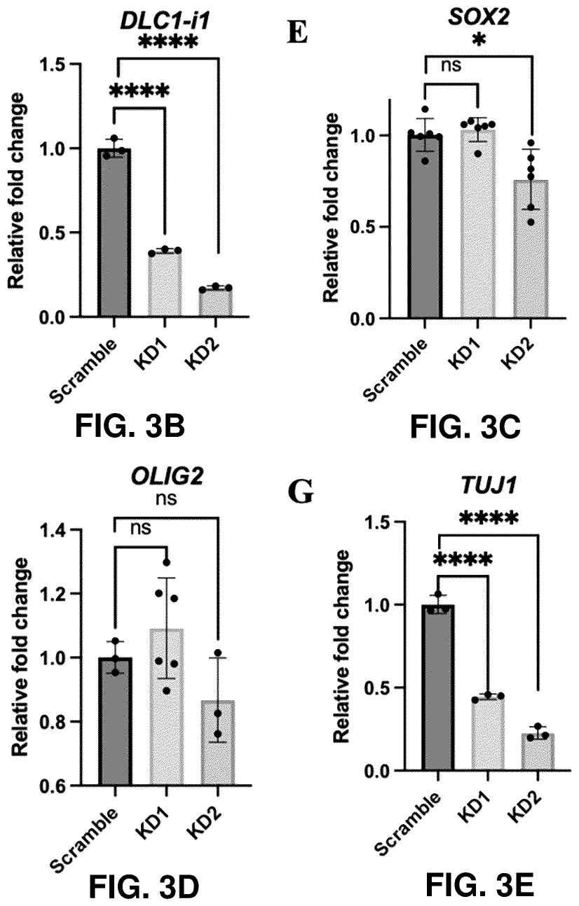

To examine whether DLC1-i1 is required for MN differentiation, shRNA lentivirus mediated knockdown (KD) of DLC1-i1 was performed in H9 ESCs followed by differentiating into OLIG2+ MNPs and post-mitotic MNs; and expression of MN markers was examined at MNPs and 7, 21 days post-mitotic MNs by immunofluorescence staining and qRT-PCR (FIG. 3A). Two shRNA oligos (KD1, KD2) were designed to specifically target the N-terminal region of DLC1-i1 and DLC1 isoform 3 (truncated and non-functional) mRNAs, both of which share the common sequence without targeting DLC1-isoform 2, 4 and 5. After lentivirus infection, expression of pluripotency markers, SOX2, OCT3/4, and NANOG, were not altered in H9 hESCs confirmed by immunofluorescence staining. Immunofluorescence staining also showed no change of motor neuron progenitor's markers: SOX2, OLIG2 and no increase of apoptosis marker: cleaved CASPASE3 after DLC1-i1 KD.

At the MNPs stage, DLC1-i1 expression in DLC1-i1 KD was significantly down-regulated by 55% and 80% from KD1 and KD2 compared to scramble control (FIG. 3B). Meanwhile, the expression of neural stem cell marker SOX2 and MNP marker (OLIG2) remained unaltered (FIGS. 3C-3D). In contrast, expression of neuron axonal marker TUJ1 was reduced to 50% and 20% in the KD1 and KD2 groups (FIG. 3E), implying reduced neuronal differentiation by DLC1-i1 KD. These results suggest that DLC1-i1 is not required for the specification of MNPs but is crucial for neuronal differentiation.

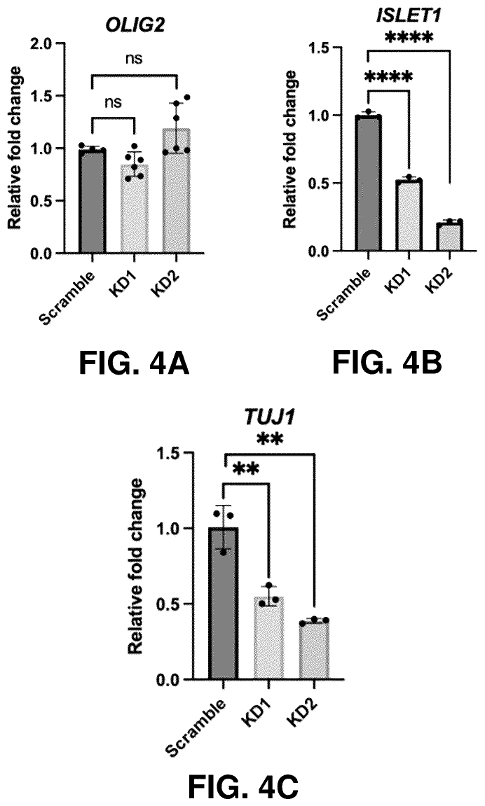

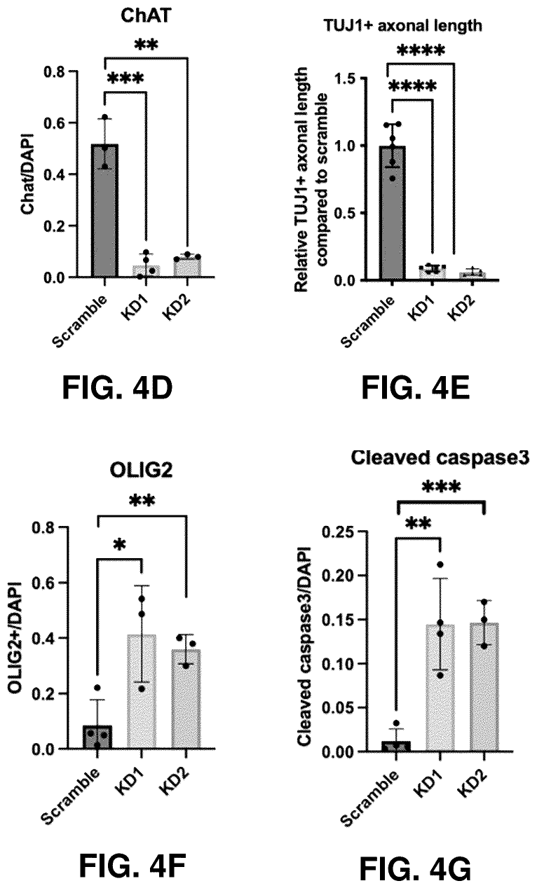

After 7 days differentiation, immunofluorescence staining showed shorter TUJ1+ axons and fewer ISLET1/2+ MNs in DLC1-i1 KD group at 7 days differentiating MNs and no change of SOX2, OLIG2 and cleaved CASPASE3. qRT-PCR confirmed that expression of SOX2 and OLIG2 remained unaltered (FIG. 4A). In contrast, expression of post-mitotic MN marker ISLET1/2 was reduced to 50% and 20% (FIG. 4B) and neuronal fibers marker TUJ1 was reduced to 50% and 40% (FIG. 4C) in DLC1-i1 KD1 and KD2 cells compared to scramble control. These results show that DLC1-i1 KD inhibit MN differentiation. Immunofluorescence staining with TUJ1 showed shorter, thinner, and twisted axons in DLC1-i1 KDs while scramble showed longer, more robust, and elongated axons. These data suggest that DLC1-i1 is required for MN differentiation and axonal outgrowth.

After 21 days differentiation, immunofluorescence staining showed shorter TUJ1+ axons, fewer CHAT+ mature MNs, decreased expression of synaptophysin (SYP) and ISLET1/2 but increased expression of OLIG2 and cleaved CASPASE3 in DLC1-i1 KD groups at 21 days differentiating MNs. qRT-PCR confirmed that mature motor neuron marker ChAT was significantly reduced in DLC1-i1 KD MNs compared to scramble (FIG. 4D). Besides, both OLIG2 and cleaved CASPASE 3 expression were up-regulated in DLC1-i1 KD MNs (FIG. 4F-4G) by staining and quantification, indicating DLC1-i1 KD led to differentiation blockade at the MNPs stage and apoptosis of differentiating MNs. By measuring the neuronal length, KD group showed marked reduction in axonal length to 10% (FIG. 4E) when compared to scramble indicating that axonal extension is severely affected by the loss of DLC1-i1. These data suggest that DLC1-i1 plays a vital role in motor neuron differentiation and survival.

### Example 4: Rescue of SMA Patients' UiPSCs Derived NMOs by DLC1-i1 Overexpression

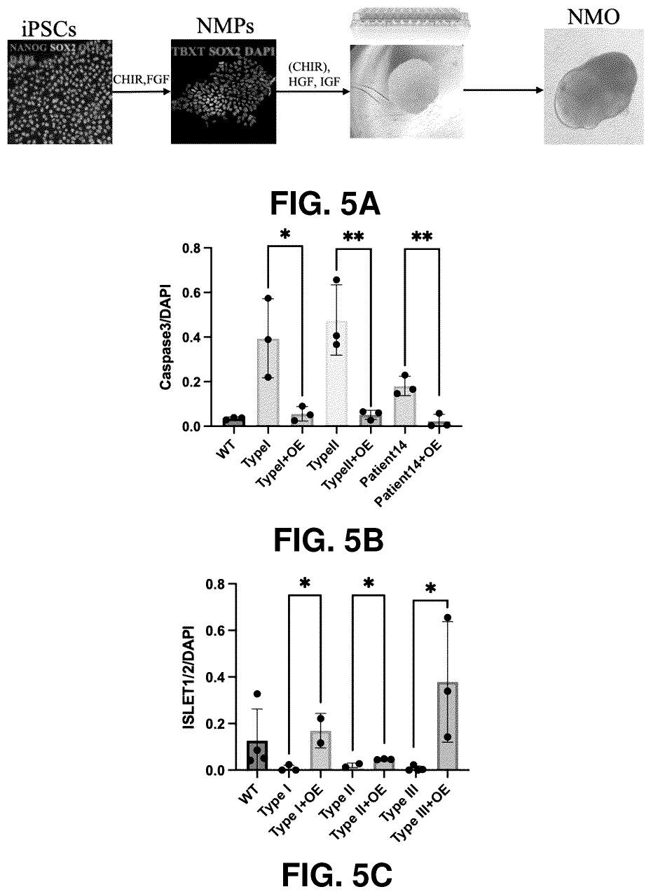

SMA is characterized by atrophy of muscles caused by degeneration of motor neurons, but the underlying mechanism is still unclear. The main obstacle is the lack of an excellent in vitro model to investigate the interaction between motor neurons and muscles. NMO including neural and muscles lineage can be simultaneously generated from NMPs (E. Tzouanacou, A. Wegener, F. J. Wymeersch, V. Wilson, J.-F. Nicolas, Developmental Cell 17, 365-376 (2009)) and is self-organized in minimal medium, serving as an appropriate model to investigate neuromuscular junctions and SMA pathogenesis. After NMPs formation, single cell suspension was centrifuged in U-shape wells to form small aggregates. Then these NMOs were treated with HGF and IGF for 3 days, followed by basal medium without growth factors for 50 days after NMPs stage (FIG. 5A). To verify whether DLC1-i1 can rescue NMJs in the SMA patients derived NMOs, DLC1-i1 OE lentivirus was added in the NMPs stage and examined at 50 days NMOs. By immunofluorescence staining, SMA patients' derived NMOs showed fewer TUJ1+ axons, ISLET1/2+ MNs, MHC+ muscle formation and elevated expression of cleaved CASPASE3. When overexpressing DLC1-i1 in patients' NMOs, there are more TUJ1+ axons, ISLET1/2+ MNs, MHC+ muscles, and fewer cleaved CASPASE3+ cells in the rescued NMOs. CASPASE3+ and ISLET1/2+ cell number quantification showed DLC1-i1 OE can significantly decrease CASPASE3+ apoptosis and increase ISLET1/2+ MNs (FIGS. 5B-5C). In summary, DLC1-i1 OE can partly rescue neuronal formation and apoptosis in SMA patients' derived NMOs.

### Example 5: Gene Therapy of SMNA7;SMN2;Smn−/− Mice by AAV-SYN-DLC1-i1

Improved Survival, Weight, and Locomotion Ability after DLC1-i1 Gene Therapy

Moderate type II SMA mice (SMNΔ7;SMN2;Smn^{-/-} mice) were purchased from the Jackson Laboratory and used as SMA in vivo model to evaluate the beneficial effect of DLC1-i1 as a gene therapy. Intracerebroventricular (ICV) injection of AAV-SYN (SYNAPSIN)-DLC1-i1 was performed into the neuronal population of PO SMA mice with AAV-SYN-SMN1 and AAV-GFP as a positive and a negative control respectively, followed by recording their lifespans and locomotion abilities such as surface righting, ambulation, hindlimb foot angle, and four limbs hanging on a daily basis. Since different dosages could result in different therapeutic effects, we adopted high dosage (1×10¹¹ vg), middle dosage (5×10¹⁰ vg), and low dosage (2.2×10¹⁰ vg) treatments for DLC1-i1 gene and SMN1 gene. Three dosages of AAV-SYN-SMN1 and AAV-SYN-DLC1-i1 treated SMN^{-/-} mice showed a significant increase of lifespan (Table 1, FIGS. 6A-6C), shorter righting time (FIGS. 6D-6F), higher ambulation scores (FIGS. 6G-6I) and longer four limbs hanging time (FIGS. 6J-6L) compared to AAV-GFP treated SMN^{-/-} mice. Although AAV9-SMN1 is a commercial drug for SMA patients, hepatotoxicity was reported in SMA patients after administration of AAV9-SMN1 (D. Chand et al., J Hepatol 74, 560-566 (2021)). Since loss of DLC1-i1 expression could partly mediate MN defects in SMA, it was examined whether gene therapy of DLC1-i1 could exhibit better or faster beneficial effect in locomotor recovery with reduced liver damage than the current drug.

To this end, the lifespan and locomotor ability of AAV-SYN-SMN1 and AAV-SYN-DLC1-i1 treated SMN^{-/-} mice were compared. Kaplan-Meier survival curves show prolonged survival of SMA mice after AAV-SYN-SMN1 and AAV-SYN-DLC1-i1 treatment in low, middle, and high dosages than GFP treatment and AAV-SYN-DLC1-i1 middle and high dosage treated mice showed a longer survival time than AAV-SYN-SMN1 treated mice (P<0.05 by Log-rank (mantel-cox) test) (FIGS. 6A-6C). The lifespans of middle dosage (5×10¹⁰ vg) AAV-SYN-DLC1-i1 treated mice are 40, 42 and 49 days, with a mean of 42.7 days. However, SMN^{-/-} mice (n=5) treated with middle dosages (5×10¹⁰ vg) of AAV-SYN-SMN1 survived for 17, 28, 30, 35, and 41 days, with 30.2 day mean lifespan. AAV-SYN-DLC1-i1 treated mice showed prolonged survival than AAV-SYN-SMN1 treated mice with a significant P<0.05 by Log-rank (mantel-cox) test. The lifespan of high dosage (1×10¹¹ vg) AAV-SYN-DLC1-i1 treated mice is 36, 46 and 49 days, with a mean of 43.7 days. High dosage (1×10¹¹ vg) AAV-SYN-SMN1 treated mice survived for 8, 17, and 26 days, with 17 days mean lifespan, which is significantly lower than the lifespan of the same dosage of DLC1-i1 treated mice (P<0.05 by Log-rank (mantel-cox) test and Gehan-Breslow-Wilcoxon test).

The locomotor ability is evaluated by righting test, ambulation scores and four limbs hanging time. Shorter righting time in SMA mice treated with AAV-SYN-SMN1 and AAV-SYN-DLC1-i1 than GFP treated. SMN^{-/-} mice treated with AAV-SYN-DLC1-i1 showed faster righting time than AAV-SYN-SMN1 treated group in medium dosage (P<0.05, two-way ANOVA, Mixed effect model (REML)) (FIGS. 6D-6F). Higher ambulation score in SMA mice treated with AAV-SYN-SMN1 and AAV-SYN-DLC1-i1 than GFP treated (P<0.05, two-way ANOVA, Mixed effect model (REML)). Moreover, SMN^{-/-} mice treated with AAV-SYN-DLC1-i1 showed higher ambulation score at day 8 or day 9 compared to AAV-SYN-SMN1 treated group (FIGS. 6G-6I, P<0.05, unpaired two-tailed student's t-test). Longer four limbs hanging time in SMA mice treated with AAV-SYN-SMN1 and AAV-SYN-DLC1-i1 than GFP treated (P<0.05, two-way ANOVA, Mixed effect model (REML)). Longer hanging time was found in SMN^{-/-} mice treated with AAV-SYN-DLC1-i1 compared to AAV-SYN-SMN1 treated mice in low, medium, and high dosage (P<0.05, unpaired two-tailed student's t-test) (FIGS. 6J-6L). These behavior tests showed that AAV-SYN-DLC1-i1 is more effective than AAV-SYN-SMN1 in improving mice locomotion and muscle strength, especially the significant longer hanging time in all three dosages of DLC1-i1 compared to SMN1. Schematics of the AAV vectors used to deliver DLC1-i1 and SMN1 are shown in FIGS. 6M and 6N, respectively.

TABLE 1
Lifespan of AAV-GFP, AAV-SYN-SMN1 and
AAV-SYN-DLC1-i1 treated SMN^{-/-} mice.

| | Low Dosage | | Middle Dosage | | High Dosage | |
|---|---|---|---|---|---|---|
| | (2.2 × 10¹⁰ vg) | | (2.2 × 10¹⁰ vg) | | (2.2 × 10¹⁰ vg) | |
| | Survival (Days) | | Survival (Days) | | Survival (Days) | |
| AAV | Mean | Longest | Mean | Longest | Mean | Longest |
| GFP | — | — | 12.7 | 15 | — | — |
| | | | (n = 6) | | | |
| SYN- | 23.6 | 35 | 30.2 | 41 | 17 | 26 |
| SMN1 | (n = 3) | | (n = 5) | | (n = 3) | |
| SYN- | 15.3 | 20 | 42.7 | 46 | 43.7 | 49 |
| DLC1-i1 | (n = 2) | | (n = 3) | | (n = 3) | |

### Example 6: AAV-SYN-SMN1 and AAV-SYN-DLC1-i1 Showed Greater than Additive Effects on Increasing Lifespan and Locomotor Ability of SMN−/− Mice

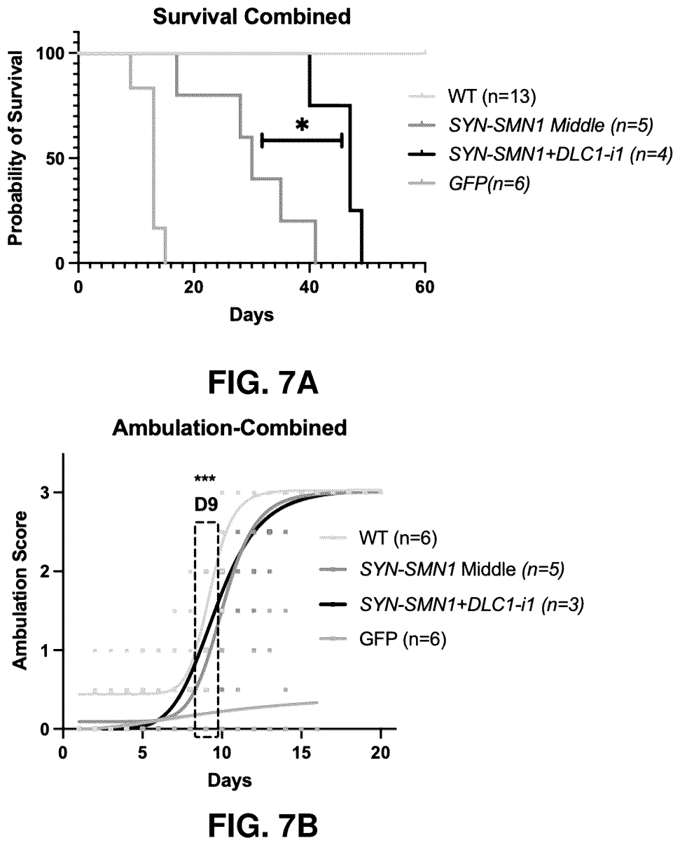

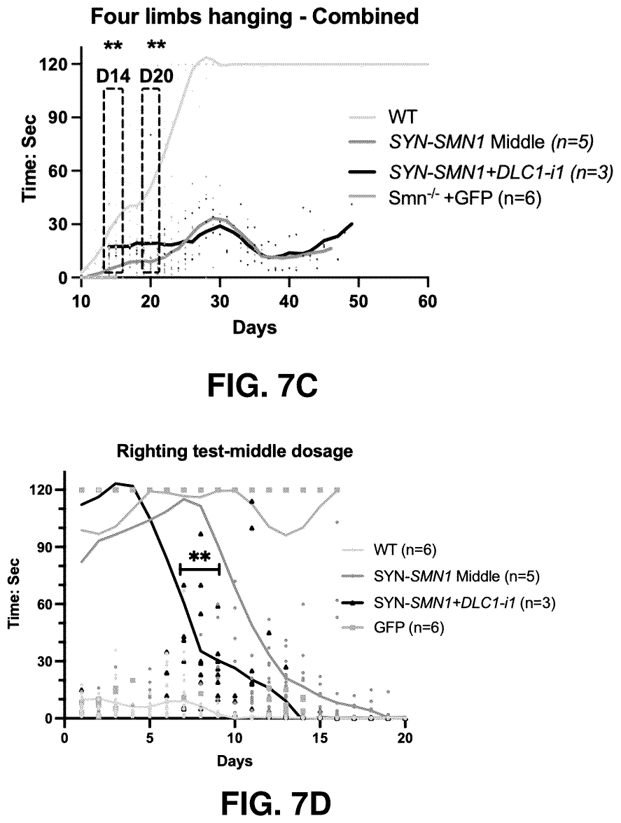

Based on the limitations in improving SMA patients' locomotion ability of current existing drug Zolgensma AAV9-SMN1 and the high efficiency of DLC1-i1 to rescue of SMN^{-/-} mouse, it was investigated whether combined injection of AAV-CAG(ubiquitous promoter)-SMN1/AAV-CAG-DLC1-i1, and AAV-SYN-SMN1/AAV-SYN-DLC1-i1 could result in synergistic effects on locomotor recovery and extension of lifespan. Survival results showed the lifespan of AAV-CAG-SMN1 and AAV-CAG-DLC1 combined treated mice can reach 59 days with a median of 48 days. AAV-SYN-SMN1 and AAV-SYN-DLC1 combined treated mice can reach 50 days with a median of 46 days (FIG. 7A, Table 2). These results demonstrated that AAV-CAG-SMN1/AAV-CAG-DLC1-i1 combination, and AAV-SYN-SMN1/AAV-SYN-DLC1-i1 can significantly improve the survival of SMA mice, which is longer than the lifespan of low dosage (2.2×10¹⁰ vg), middle dosage (5×10¹⁰ vg) SMN1 or middle (5×10¹⁰ vg) dosage DLC1-i1 treatment alone shown above (P<0.05 by Log-rank (mantel-cox) test). These results show the greater than additive effect of AAV-PHP.eB SYN-SMN1 and AAV-PHP.eB SYN-DLC1-i1 on enhancing survival of SMA mice compared to treatment alone.

Higher ambulation score in SMA mice treated with AAV-PHP.eB SYN-SMN1+SYN-DLC1-i1 than GFP treated. Ambulation score showed AAV-PHP.eB SYN-SMN1 and AAV-PHP.eB SYN-DLC1-i1 combined group exhibited higher score than middle dosage AAV-PHP.eB SYN-SMN1 treated group in day 9 (FIG. 7B). Longer four limbs hanging time in SMN^{-/-} mice treated with AAV-PHP.eB SYN-SMN1+SYN-DLC1-i1 than GFP treated (P<0.05, two-way ANOVA, Mixed effect model (REML)). Combined group also showed longer hanging time at early stage (day 14 and day 20) than middle dosage AAV-PHP.eB SYN-SMN1 treated group (FIG. 7C). Shorter righting time was observed in SMN^{-/-} mice treated with AAV-PHP.eB SYN-SMN1+SYN-DLC1-i1 than GFP treated or AAV-PHP.eB SYN-SMN1 middle dosage treated mice (FIG. 7D). This result shows combined treatment can improve locomotor ability of SMA mice at earlier stage than AAV-PHP.eB SMN1 alone. In summary, AAV-PHP.eB SYN-SMN1 and AAV-PHP.eB SYN-DLC1-i1 combination treatment has a synergistic effect on improving survival and locomotor ability of SMA mice.

TABLE 2
Lifespan of AAV-PHP.eB SMN1 and AAV-PHP.eB DLC1-i1
combination treated SMN^{—/—} mice.

AAV-PHP.eB

| | | |
|---|---|---|
| Low dosage | Medium dosage | Survival (Days) |

| (2.2 × 10¹⁰ vg) | (5 × 10¹⁰ vg) | Mean | Longest |
|---|---|---|---|
| CAG-SMN1 | CAG-DLC1-i1 | 48 (n = 3) | 59 |
| SYN-SMN1 | SYN-DLC1-i1 | 46 (n = 3) | 50 |

We claim:

1. A recombinant AAV viral particle comprising a vector encoding a Deleted in Liver Cancer 1-isoform 1 (DLC1-i1) transgene, or the expression product thereof.

2. The recombinant AAV viral particle of claim 1, wherein the DLC1-i1 transgene is operably linked to a promoter selected from the group consisting of the human synapsin 1 gene promoter (SYN), human β-glucuronidase promoter and a cytomegalovirus enhancer linked to a chicken β-actin promoter.

3. The recombinant AAV viral particle of claim 1, wherein the DLC1-i1 transgene is operably linked to the human synapsin 1 gene promoter (SYN).

4. The recombinant AAV viral particle of claim 1, wherein the vector comprises the nucleic acid sequence of SEQ ID NO: 1 and/or SEQ ID NO:2, and/or a polynucleotide encoding the polypeptide having an amino acid sequence of SEQ ID NO:3.

5. The recombinant AAV viral particle of claim 1, wherein the AAV viral particle comprises an AAV1, AAV2, AAV3, AAV4, AAV5, AAV6, AAV7, AAVS, AAVrh8, AAV9, AAV10, AAVrh10, AAV11, or AAV12 serotype capsid.

6. The recombinant AAV viral particle of claim 5, wherein the rAAV viral particle comprises an AAV9 serotype capsid.

7. The recombinant AAV viral particle of claim 6, wherein the AAV viral particle comprises an AAV serotype capsid from Clades A-F.

8. The recombinant AAV viral particle of claim 7, wherein, wherein the vector comprises AAV1, AAV2, AAV3, AAV4, AAV5, AAV6, AAV7, AAVS, AAVrh8, AAV9, AAV10, AAVrh10, AAV11, or AAV12 serotype inverted terminal repeats (ITRs).

9. The recombinant AAV viral particle of claim 1, wherein the vector comprises AAV serotype 2 ITRs.

10. The recombinant AAV viral particle of claim 1, further comprising a survival motor neuron 1 (SMN1) transgene,
wherein the SMN1 transgene is within the same or different vector as the DLC1-i1 transgene.

11. The recombinant AAV viral particle of claim 10, wherein the SMN1 transgene is operably linked to the same or different promoter as the DLC1-i1 transgene.

12. The recombinant AAV viral particle of claim 10, wherein the recombinant AAV viral particle of claim 1, wherein the SMN1 transgene is operably linked to the human synapsin 1 gene promoter (SYN).

13. A composition for administration to a subject in vivo, comprising
(a) the recombinant AAV viral particle of claim 1; and
(b) a pharmaceutically acceptable excipient for administration.

14. The composition of claim 13, further comprising
(c) a recombinant AAV viral particle comprising a survival motor neuron 1 (SMN1) transgene, optionally wherein the SMN1 transgene is operably linked to a synapsin 1 gene promoter (SYN).

15. The composition of claim 13, comprising the recombinant AAV viral particles in an amount of between about 2.5×10¹² genome copies and about 5×10¹³ genome copies.

16. The composition of claim 13, comprising the recombinant AAV viral particle in an amount of least 5×10¹³ genome copies per kg body weight of the subject.

17. A method for treating spinal muscular atrophy (SMA) in a subject, comprising administering to the subject a first composition comprising the composition of claim 13.

18. The method of claim 17, wherein the method ameliorates or minimizes one or more symptoms of SMA in the subject, wherein the one or more symptom is selected from the group consisting of muscle wasting, inability to achieve motor milestones, inability to sit, inability to walk, paralysis, respiratory dysfunction, bulbar dysfunction, motor neuron cell loss and neuromuscular junction pathology.

19. The method of claim 17, wherein the composition comprises at least 1×10¹² genome copies of recombinant AAV comprising a DLC1-i1 transgene.

20. The method of claim 17, wherein the methods administer at least 3.5×10¹¹ genome copies per kg body weight of recombinant AAV viral particles comprising a DLC1-i1 transgene to the subject.

21. The method of claim 17, wherein at least 10-30% of motor neurons in the lumbar, thoracic and cervical regions of the spinal cord of the subject are transduced by the recombinant AAV viral particles.

22. The method of claim 17, wherein at least 30% of wild type level of DLC1-i1 is generated throughout the spinal cord.

23. The method of claim 17, wherein the composition is administered to the subject via intravenous injection, or via direct injection into the spinal cord, or via intrathecal injection, or via intracisternal injection.

24. The method of claim 17, wherein the composition is administered to more than one location of the spinal cord or cisterna magna.

25. The method of claim 24, wherein the composition is administered to more than one location of the spinal cord.

26. The method of claim 17, wherein the composition is administered to one or more of a lumbar subarachnoid space, thoracic subarachnoid space and a cervical subarachnoid space of the spinal cord.

27. The method of claim 26, wherein the composition is administered to the cisterna magna.

28. The method of claim 17, further comprising administering to the subject a second composition comprising a recombinant AAV viral particle comprising a survival motor neuron 1 (SMN1) transgene, optionally wherein the SMN1 transgene is operably linked to a synapsin 1 gene promoter (SYN).

29. The method of claim 28, wherein the SMN1 transgene comprises the polynucleotide of SEQ ID NO:4 and/or encodes a polypeptide having the amino acid sequence of SEQ ID NO:5.

30. The method of claim 28, wherein the second composition is administered to the subject at the same time as, before or after the first composition.

31. The method of claim 28, wherein the therapeutic effect of the administering the first and second compositions to the subject is greater than the additive effects of administering the first composition alone or the second composition alone.

32. The method of claim 28, wherein the methods administer at least 3.5×10¹¹ genome copies per kg body weight of recombinant AAV viral particles comprising a SMN1 transgene to the subject.

33. The method of claim 28, wherein the subject is a pediatric subject.

34. The method of claim 28, wherein the subject is a young adult.

35. The method of claim 28, wherein the subject has spinal muscular atrophy, optionally wherein the subject has a mutation in the endogenous DLC1-i1 gene, and/or the SMN-1 gene.

36. The method of claim 35, wherein the subject has a partial deletion of the endogenous DLC1-i1 gene, and/or the SMN1 gene.

37. The method of claim 35, wherein the subject has a complete deletion of the endogenous DLC1-i1 gene, and/or the SMN1 gene.

38. The method of claim 35, wherein expression of the mutant DLC1-i1 gene, and/or the SMN-1 gene in spinal cord or brain of the subject is deficient compared to expression of DLC1-i1, and/or SMN-1 in a subject with a wild-type DLC1-i1 gene, and/or SMN-1 gene.

39. A recombinant viral particle comprising the nucleic acid sequence of SEQ ID NO:10 or 11.

40. The recombinant viral particle of claim 39, comprising the nucleic acid sequence of SEQ ID NO:10.

41. A cell comprising the recombinant viral particle of claim 40.

42. The cell of claim 41, further comprising the nucleic acid sequence of SEQ ID NO:11.
